SEQUENCE LISTING (344 sequences)

SEQ ID NO: 1 - protein, 15 aa, synthetic construct
KASQSVDYDGDSYMN

SEQ ID NO: 2 - protein, 7 aa, synthetic construct
GASNLES

SEQ ID NO: 3 - protein, 9 aa, synthetic construct
QQSIEDPWT

SEQ ID NO: 4 - protein, 6 aa, synthetic construct
GGYNWH

SEQ ID NO: 5 - protein, 16 aa, synthetic construct
DIHKSGSTNYNPSLKS

SEQ ID NO: 6 - protein, 11 aa, synthetic construct
DYYGFSWYFDV

SEQ ID NO: 7 - protein, 111 aa, synthetic construct
DIVLTQSPASLAVSLGQRATISCKASQSVDYDGDSYMNWYQQKPGQPPKLLIYGASNLESGIPARFSGSGSGRDFTLNIHPVEEEDAATYYCQQSIEDPWTFGGGTKLEIK

SEQ ID NO: 8 - protein, 120 aa, synthetic construct
DVQLQESGPDLVKPSQSLSLTCTVTGYSITGGYNWHWIRQFPGNKLEWMGDIHKSGSTNYNPSLKSRISITRDTSKNQFFLQLNSVTTEDTATYYCARDYYGFSWYFDVWGAGTTVTVSS

SEQ ID NO: 9 - protein, 15 aa, synthetic construct
KASQSVDYDGDSYMN

SEQ ID NO: 10 - protein, 7 aa, synthetic construct
GASNLES

SEQ ID NO: 11 - protein, 9 aa, synthetic construct
QQSIEDPWT

SEQ ID NO: 12 - protein, 6 aa, synthetic construct
GGYNWH

SEQ ID NO: 13 - protein, 16 aa, synthetic construct
DIHKSGSTNYNPSLKS

SEQ ID NO: 14 - protein, 11 aa, synthetic construct
DYYGFSWYFDV

SEQ ID NO: 15 - protein, 111 aa, synthetic construct
DIVLTQSPDSLAVSLGERATINCKASQSVDYDGDSYMNWYQQKPGQPPKLLIYGASNLESGIPDRFSGSGSGRDFTLTISSLQAEDVATYYCQQSIEDPWTFGGGTKLEIK

SEQ ID NO: 16 - protein, 120 aa, synthetic construct
QVQLQESGPGLVKPSQTLSLTCTVTGYSITGGYNWHWIRQPPGKGLEWMGDIHKSGSTNYNPSLKSRITISRDTSKNQFSLKLSSVTAADTAVYYCARDYYGFSWYFDVWGQGTTVTVSS

SEQ ID NO: 17 - protein, 15 aa, synthetic construct
KASQSVDYDGDSYMN

SEQ ID NO: 18 - protein, 7 aa, synthetic construct
GASNRES

SEQ ID NO: 19 - protein, 9 aa, synthetic construct
QQSIEDPWT

SEQ ID NO: 20 - protein, 6 aa, synthetic construct
GGYNWH

SEQ ID NO: 21 - protein, 16 aa, synthetic construct
DIHKSGSTNYNPSLKS

SEQ ID NO: 22 - protein, 11 aa, synthetic construct
DYYGFSWYFDV

SEQ ID NO: 23 - protein, 111 aa, synthetic construct
DIVLTQSPDSLAVSLGERATINCKASQSVDYDGDSYMNWYQQKPGQPPKLLIYGASNRESGVPDRFSGSGSGRDFTLTISSLQAEDVAVYYCQQSIEDPWTFGGGTKLEIK

SEQ ID NO: 24 - protein, 120 aa, synthetic construct; identical to SEQ ID NO: 16

SEQ ID NO: 25 - protein, 15 aa, synthetic construct
RASQSVDYDGDSYMN

SEQ ID NO: 26 - protein, 7 aa, synthetic construct
GASNLES

SEQ ID NO: 27 - protein, 9 aa, synthetic construct
QQSIEDPWT

SEQ ID NO: 28 - protein, 6 aa, synthetic construct
GGYNWH

SEQ ID NO: 29 - protein, 16 aa, synthetic construct
DIHKSGSTNYNPSLKS

SEQ ID NO: 30 - protein, 11 aa, synthetic construct
DYYGFSWYFDV

SEQ ID NO: 31 - protein, 111 aa, synthetic construct
DIVLTQSPATLSLSPGERATLSCRASQSVDYDGDSYMNWYQQKPGQAPRLLIYGASNLESGIPARFSGSGSGRDFTLTISSLEPEDFATYYCQQSIEDPWTFGGGTKLEIK

SEQ ID NO: 32 - protein, 120 aa, synthetic construct; identical to SEQ ID NO: 16

SEQ ID NO: 33 - protein, 15 aa, synthetic construct
RASQSVDYDGDSYMN

SEQ ID NO: 34 - protein, 7 aa, synthetic construct
GASNRET

SEQ ID NO: 35 - protein, 9 aa, synthetic construct
QQSIEDPWT

SEQ ID NO: 36 - protein, 6 aa, synthetic construct
GGYNWH

SEQ ID NO: 37 - protein, 16 aa, synthetic construct
DIHKSGSTNYNPSLKS

SEQ ID NO: 38 - protein, 11 aa, synthetic construct
DYYGFSWYFDV

SEQ ID NO: 39 - protein, 111 aa, synthetic construct
EIVLTQSPATLSLSPGERATLSCRASQSVDYDGDSYMNWYQQKPGQAPRLLIYGASNRETGIPARFSGSGSGRDFTLTISSLEPEDFAVYYCQQSIEDPWTFGGGTKLEIK

SEQ ID NO: 40 - protein, 120 aa, synthetic construct; identical to SEQ ID NO: 16

SEQ ID NO: 41 - protein, 15 aa, synthetic construct
KASQSVDYDGDSYMN

SEQ ID NO: 42 - protein, 7 aa, synthetic construct
GASNLES

SEQ ID NO: 43 - protein, 9 aa, synthetic construct
QQSIEDPWT

SEQ ID NO: 44 - protein, 6 aa, synthetic construct
GGYNWH

SEQ ID NO: 45 - protein, 16 aa, synthetic construct
DIHKSGSTNYNPSLKS

SEQ ID NO: 46 - protein, 11 aa, synthetic construct
DYYGFSWYFDV

SEQ ID NO: 47 - protein, 111 aa, synthetic construct; identical to SEQ ID NO: 15

SEQ ID NO: 48 - protein, 120 aa, synthetic construct
QVQLQESGPGLVKPSQTLSLTCTVTGYSITGGYNWHWIRQPPGKGLEWIGDIHKSGSTNYNPSLKSRVTISRDTSKNQFSLKLSSVTAADTAVYYCARDYYGFSWYFDVWGQGTTVTVSS

SEQ ID NO: 49 - protein, 15 aa, synthetic construct
KASQSVDYDGDSYMN

SEQ ID NO: 50 - protein, 7 aa, synthetic construct
GASNRES

SEQ ID NO: 51 - protein, 9 aa, synthetic construct
QQSIEDPWT

SEQ ID NO: 52 - protein, 6 aa, synthetic construct
GGYNWH

SEQ ID NO: 53 - protein, 16 aa, synthetic construct
DIHKSGSTNYNPSLKS

SEQ ID NO: 54 - protein, 11 aa, synthetic construct
DYYGFSWYFDV

SEQ ID NO: 55 - protein, 111 aa, synthetic construct; identical to SEQ ID NO: 23

SEQ ID NO: 56 - protein, 120 aa, synthetic construct; identical to SEQ ID NO: 48

SEQ ID NO: 57 - protein, 15 aa, synthetic construct
RASQSVDYDGDSYMN

SEQ ID NO: 58 - protein, 7 aa, synthetic construct
GASNLES

SEQ ID NO: 59 - protein, 9 aa, synthetic construct
QQSIEDPWT

SEQ ID NO: 60 - protein, 6 aa, synthetic construct
GGYNWH

SEQ ID NO: 61 - protein, 16 aa, synthetic construct
DIHKSGSTNYNPSLKS

SEQ ID NO: 62 - protein, 11 aa, synthetic construct
DYYGFSWYFDV

SEQ ID NO: 63 - protein, 111 aa, synthetic construct; identical to SEQ ID NO: 31

SEQ ID NO: 64 - protein, 120 aa, synthetic construct; identical to SEQ ID NO: 48

SEQ ID NO: 65 - protein, 15 aa, synthetic construct
RASQSVDYDGDSYMN

SEQ ID NO: 66 - protein, 7 aa, synthetic construct
GASNRET

SEQ ID NO: 67 - protein, 9 aa, synthetic construct
QQSIEDPWT

SEQ ID NO: 68 - protein, 6 aa, synthetic construct
GGYNWH

SEQ ID NO: 69 - protein, 16 aa, synthetic construct
DIHKSGSTNYNPSLKS

SEQ ID NO: 70 - protein, 11 aa, synthetic construct
DYYGFSWYFDV

SEQ ID NO: 71 - protein, 111 aa, synthetic construct; identical to SEQ ID NO: 39

SEQ ID NO: 72 - protein, 120 aa, synthetic construct; identical to SEQ ID NO: 48

SEQ ID NO: 73 - protein, 15 aa, synthetic construct
KASQSVDYDGDSYMN

SEQ ID NO: 74 - protein, 7 aa, synthetic construct
GASNLES

SEQ ID NO: 75 - protein, 9 aa, synthetic construct
QQSIEDPWT

SEQ ID NO: 76 - protein, 6 aa, synthetic construct
GGYNWH

SEQ ID NO: 77 - protein, 16 aa, synthetic construct
DIHKSGSTNYNPSLKT

SEQ ID NO: 78 - protein, 11 aa, synthetic construct
DYYGFSWYFDV

SEQ ID NO: 79 - protein, 111 aa, synthetic construct; identical to SEQ ID NO: 15

SEQ ID NO: 80 - protein, 120 aa, synthetic construct
QVTLKESGPALVKPTQTLTLTCTVTGYSITGGYNWHWIRQPPGKALEWMGDIHKSGSTNYNPSLKTRITISRDTSKNQFVLTMTNMDPVDTATYYCARDYYGFSWYFDVWGQGTTVTVSS

SEQ ID NO: 81 - protein, 15 aa, synthetic construct
KASQSVDYDGDSYMN

SEQ ID NO: 82 - protein, 7 aa, synthetic construct
GASNRES

SEQ ID NO: 83 - protein, 9 aa, synthetic construct
QQSIEDPWT

SEQ ID NO: 84 - protein, 6 aa, synthetic construct
GGYNWH

SEQ ID NO: 85 - protein, 16 aa, synthetic construct
DIHKSGSTNYNPSLKT

SEQ ID NO: 86 - protein, 11 aa, synthetic construct
DYYGFSWYFDV

SEQ ID NO: 87 - protein, 111 aa, synthetic construct; identical to SEQ ID NO: 23

SEQ ID NO: 88 - protein, 120 aa, synthetic construct; identical to SEQ ID NO: 80

SEQ ID NO: 89 - protein, 15 aa, synthetic construct
RASQSVDYDGDSYMN

SEQ ID NO: 90 - protein, 7 aa, synthetic construct
GASNLES

SEQ ID NO: 91 - protein, 9 aa, synthetic construct
QQSIEDPWT

SEQ ID NO: 92 - protein, 6 aa, synthetic construct
GGYNWH

SEQ ID NO: 93 - protein, 16 aa, synthetic construct
DIHKSGSTNYNPSLKT

SEQ ID NO: 94 - protein, 11 aa, synthetic construct
DYYGFSWYFDV

SEQ ID NO: 95 - protein, 111 aa, synthetic construct; identical to SEQ ID NO: 31

SEQ ID NO: 96 - protein, 120 aa, synthetic construct; identical to SEQ ID NO: 80

SEQ ID NO: 97 - protein, 15 aa, synthetic construct
RASQSVDYDGDSYMN

SEQ ID NO: 98 - protein, 7 aa, synthetic construct
GASNRET

SEQ ID NO: 99 - protein, 9 aa, synthetic construct
QQSIEDPWT

SEQ ID NO: 100 - protein, 6 aa, synthetic construct
GGYNWH

SEQ ID NO: 101 - protein, 16 aa, synthetic construct
DIHKSGSTNYNPSLKT

SEQ ID NO: 102 - protein, 11 aa, synthetic construct
DYYGFSWYFDV

SEQ ID NO: 103 - protein, 111 aa, synthetic construct; identical to SEQ ID NO: 39

SEQ ID NO: 104 - protein, 120 aa, synthetic construct; identical to SEQ ID NO: 80

SEQ ID NO: 105 - protein, 15 aa, synthetic construct
KASQSVDYDGDSYMN

SEQ ID NO: 106 - protein, 7 aa, synthetic construct
GASNLES

SEQ ID NO: 107 - protein, 9 aa, synthetic construct
QQSIEDPWT

SEQ ID NO: 108 - protein, 6 aa, synthetic construct
GGYNWH

SEQ ID NO: 109 - protein, 16 aa, synthetic construct
DIHKSGSTNYNPSLKT

SEQ ID NO: 110 - protein, 11 aa, synthetic construct
DYYGFSWYFDV

SEQ ID NO: 111 - protein, 111 aa, synthetic construct; identical to SEQ ID NO: 15

SEQ ID NO: 112 - protein, 120 aa, synthetic construct
QVTLKESGPALVKPTQTLTLTCTVTGYSITGGYNWHWIRQPPGKALEWIGDIHKSGSTNYNPSLKTRLTISRDTSKNQFVLTMTNMDPVDTATYYCARDYYGFSWYFDVWGQGTTVTVSS

SEQ ID NO: 113 - protein, 15 aa, synthetic construct
KASQSVDYDGDSYMN

SEQ ID NO: 114 - protein, 7 aa, synthetic construct
GASNRES

SEQ ID NO: 115 - protein, 9 aa, synthetic construct
QQSIEDPWT

SEQ ID NO: 116 - protein, 6 aa, synthetic construct
GGYNWH

SEQ ID NO: 117 - protein, 16 aa, synthetic construct
DIHKSGSTNYNPSLKT

SEQ ID NO: 118 - protein, 11 aa, synthetic construct
DYYGFSWYFDV

SEQ ID NO: 119 - protein, 111 aa, synthetic construct; identical to SEQ ID NO: 23

SEQ ID NO: 120 - protein, 120 aa, synthetic construct; identical to SEQ ID NO: 112

SEQ ID NO: 121 - protein, 15 aa, synthetic construct
RASQSVDYDGDSYMN

SEQ ID NO: 122 - protein, 7 aa, synthetic construct
GASNLES

SEQ ID NO: 123 - protein, 9 aa, synthetic construct
QQSIEDPWT

SEQ ID NO: 124 - protein, 6 aa, synthetic construct
GGYNWH

SEQ ID NO: 125 - protein, 16 aa, synthetic construct
DIHKSGSTNYNPSLKT

SEQ ID NO: 126 - protein, 11 aa, synthetic construct
DYYGFSWYFDV

SEQ ID NO: 127 - protein, 111 aa, synthetic construct; identical to SEQ ID NO: 31

SEQ ID NO: 128 - protein, 120 aa, synthetic construct; identical to SEQ ID NO: 112

SEQ ID NO: 129 - protein, 15 aa, synthetic construct
RASQSVDYDGDSYMN

SEQ ID NO: 130 - protein, 7 aa, synthetic construct
GASNRET

SEQ ID NO: 131 - protein, 9 aa, synthetic construct
QQSIEDPWT

SEQ ID NO: 132 - protein, 6 aa, synthetic construct
GGYNWH

SEQ ID NO: 133 - protein, 16 aa, synthetic construct
DIHKSGSTNYNPSLKT

SEQ ID NO: 134 - protein, 11 aa, synthetic construct
DYYGFSWYFDV

SEQ ID NO: 135 - protein, 111 aa, synthetic construct; identical to SEQ ID NO: 39

SEQ ID NO: 136 - protein, 120 aa, synthetic construct; identical to SEQ ID NO: 112

SEQ ID NO: 137 - protein, 16 aa, synthetic construct
RSTQSIVHNNGNTYLE

SEQ ID NO: 138 - protein, 7 aa, synthetic construct
KVSNRFS

SEQ ID NO: 139 - protein, 9 aa, synthetic construct
FQGSHVPYT

SEQ ID NO: 140 - protein, 5 aa, synthetic construct
GNNMN

SEQ ID NO: 141 - protein, 17 aa, synthetic construct
NIDPYYGATSYNQKFKD

SEQ ID NO: 142 - protein, 9 aa, synthetic construct
STMITPFDY

SEQ ID NO: 143 - protein, 112 aa, synthetic construct
DILMTQTPLSLPVSLGDQASISCRSTQSIVHNNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 144 - protein, 118 aa, synthetic construct
EVQLQQSGPELEKPGASVKISCKASGYSFTGNNMNWVKQSNGKSLEWIGNIDPYYGATSYNQKFKDKATLTVDKSSSTAYMQLKGLTSEDSAVYYCARSTMITPFDYWGQGTTLTVSS

SEQ ID NO: 145 - protein, 16 aa, synthetic construct
RSTQSIVHNNGNTYLE

SEQ ID NO: 146 - protein, 7 aa, synthetic construct
KVSNRFS

SEQ ID NO: 147 - protein, 9 aa, synthetic construct
FQSSHVPYT

SEQ ID NO: 148 - protein, 5 aa, synthetic construct
GNNIN

SEQ ID NO: 149 - protein, 17 aa, synthetic construct
NIDPYYGATSYNQKFKG

SEQ ID NO: 150 - protein, 9 aa, synthetic construct
STMITPFDY

SEQ ID NO: 151 - protein, 112 aa, synthetic construct
DILMTQIPLSLPVSLGDQASISCRSTQSIVHNNGNTYLEWYLQKPGQSPKLLIYKVSNRFSGVPDRFSGSGSGTDFTLKISRVEAEDLGVYYCFQSSHVPYTFGGGTKLEIR

SEQ ID NO: 152 - protein, 118 aa, synthetic construct
EVQLQQSGPELEKPGASVKISCKASGYSFTGNNINWVKQSNGKSLEWIGNIDPYYGATSYNQKFKGKATLTVDKSSNTAYMQLKGLTSEDSAVYYCARSTMITPFDYWGQGTTLTVSS

SEQ ID NO: 153 - protein, 11 aa, synthetic construct
SASQGIRNHLN

SEQ ID NO: 154 - protein, 7 aa, synthetic construct
YTSSLHS

SEQ ID NO: 155 - protein, 9 aa, synthetic construct
QQYSNLPWT

SEQ ID NO: 156 - protein, 6 aa, synthetic construct
SGYDWN

SEQ ID NO: 157 - protein, 16 aa, synthetic construct
YISYDGSNNYNPSLKN

SEQ ID NO: 158 - protein, 8 aa, synthetic construct
WYYYAMDY

SEQ ID NO: 159 - protein, 107 aa, synthetic construct
DIQMTQTTSSLSASLGDRVTISCSASQGIRNHLNWYQQKPDGTVKLLIYYTSSLHSGVPSRFSGSGSGTDYSLTISNLEPEDIATYYCQQYSNLPWTFGGGTKLEIK

SEQ ID NO: 160 - protein, 117 aa, synthetic construct
DVQLQESGPGLVKPSQSLSLTCSVTGYSITSGYDWNWIRQFPGNKLEWMGYISYDGSNNYNPSLKNRISITRDTSKSQFFLKLNSVTTEDTATYYCARWYYYAMDYWGQGTSVTVSS

SEQ ID NO: 161 - protein, 16 aa, synthetic construct
RSSQSIVHSNGNTYLE

SEQ ID NO: 162 - protein, 7 aa, synthetic construct
KVSNRFS

SEQ ID NO: 163 - protein, 9 aa, synthetic construct
FQGSHVPYT

SEQ ID NO: 164 - protein, 5 aa, synthetic construct
SYGMS

SEQ ID NO: 165 - protein, 17 aa, synthetic construct
TISIGGSYTYYPDSVKG

SEQ ID NO: 166 - protein, 12 aa, synthetic construct
HSSIYDGYYGAY

SEQ ID NO: 167 - protein, 112 aa, synthetic construct
DVLMTQTPLSLPVSLGDQASISCRSSQSIVHSNGNTYLEWYLQKPGQSPKLLIYKVSNRFSGVPDRFSGSGSGTDFTLKISRVAAEDLAVYYCFQGSHVPYTFGGGTKLEIK

SEQ ID NO: 168 - protein, 121 aa, synthetic construct
EVQLVESGGDLVKPGGSLKLSCAASGFTFSSYGMSWVRQTPDKRLEWVATISIGGSYTYYPDSVKGRFTISRDNAKNTLYLQLSSLKSEDTALYYCARHSSIYDGYYGAYWGQGTLVTVSA

SEQ ID NO: 169 - protein, 15 aa, synthetic construct
RASESVDSYGNSFMH

SEQ ID NO: 170 - protein, 7 aa, synthetic construct
LASNLES

SEQ ID NO: 171 - protein, 9 aa, synthetic construct
QQNNEDPPT

SEQ ID NO: 172 - protein, 5 aa, synthetic construct
SYAIS

SEQ ID NO: 173 - protein, 16 aa, synthetic construct
SISSAGNTYYADSVKG

SEQ ID NO: 174 - protein, 14 aa, synthetic construct
GRESLLLRSYYFDY

SEQ ID NO: 175 - protein, 111 aa, synthetic construct
NIVLTQSPASLAVSLGQRATISCRASESVDSYGNSFMHWYQQKPGQPPKLLIYLASNLESGVPARFSGSGSRTDFTLTIDPVEADDAATYYCQQNNEDPPTFGGGTKLEIK

SEQ ID NO: 176 - protein, 122 aa, synthetic construct
EVKLVESGGGLVKPGGSLKVSCAASGFTFSSYAISWVRQTPEKRLEWVASISSAGNTYYADSVKGRFTISRDDARNILYLQMSSLRSEDTAIYYCARGRESLLLRSYYFDYWGQGTTLTVSS

SEQ ID NO: 177 - protein, 16 aa, synthetic construct
RSSQSIVHSNGNTYLE

SEQ ID NO: 178 - protein, 7 aa, synthetic construct
TVSNRFS

SEQ ID NO: 179 - protein, 9 aa, synthetic construct
FQGSHVPPT

SEQ ID NO: 180 - protein, 5 aa, synthetic construct
GYNMN

SEQ ID NO: 181 - protein, 17 aa, synthetic construct
NVDPYYGATSYNQKFKG

SEQ ID NO: 182 - protein, 10 aa, synthetic construct
GTYGSYAMDY

SEQ ID NO: 183 - protein, 112 aa, synthetic construct
DVLMTQTPLSLPVSLGDQASISCRSSQSIVHSNGNTYLEWYLQKPGQSPKVLIYTVSNRFSGVPDRFSGSGSGTDFTLKISRVEAEDLGVYYCFQGSHVPPTFGSGTRLEIK

SEQ ID NO: 184 - protein, 119 aa, synthetic construct
EVQLQQSGPELEKPGASVKISCKASGYSFTGYNMNWVKQSNGKSLEWIGNVDPYYGATSYNQKFKGKATLTVDKSSSTAYMQLKSLTSEDSAVYYCARGTYGSYAMDYWGQGTSVTVSS

SEQ ID NO: 185 - protein, 16 aa, synthetic construct
RSSQSILHSNGNTYLE

SEQ ID NO: 186 - protein, 7 aa, synthetic construct
KVSNRFS

SEQ ID NO: 187 - protein, 9 aa, synthetic construct
FQTSHVPWT

SEQ ID NO: 188 - protein, 5 aa, synthetic construct
SYGMS

SEQ ID NO: 189 - protein, 17 aa, synthetic construct
TINSGGSYTYYPDSVKG

SEQ ID NO: 190 - protein, 13 aa, synthetic construct
HEDYEGDYYAMNY

SEQ ID NO: 191 - protein, 112 aa, synthetic construct
DVLMTQSPLSLPVSLGDQASISCRSSQSILHSNGNTYLEWYLQKPGQSPKLLIYKVSNRFSGVPDRFSGSGSGTDFTLKISRVEAEDLGVYYCFQTSHVPWTFGGGTKLEIK

SEQ ID NO: 192 - protein, 122 aa, synthetic construct
EVQLVESGGDLVKPGGSLKLSCAASGFTFSSYGMSWVRQTPDKRLEWVATINSGGSYTYYPDSVKGRFTISRDNAKNTLYLQMSSLKSEDTAMYYCARHEDYEGDYYAMNYWGQGSSVTVSP

SEQ ID NO: 193 - protein, 13 aa, synthetic construct
SVDYYGTSLMQWY

SEQ ID NO: 194 - protein, 8 aa, synthetic construct
NVESGVPA

SEQ ID NO: 195 - protein, 9 aa, synthetic construct
QQSRKVPWT

SEQ ID NO: 196 - protein, 11 aa, synthetic construct
GYSITSDYAWN

SEQ ID NO: 197 - protein, 15 aa, synthetic construct
IIYSGSTNYNPSLES

SEQ ID NO: 198 - protein, 14 aa, synthetic construct
ARSTYGGNYWHFDV

SEQ ID NO: 199 - protein, 111 aa, synthetic construct
DIVLTQSPASLAVSLGQRATISCRASESVDYYGTSLMQWYQQKPGQPPKLLIYAASNVESGVPARFSGSGSGTDFSLNIHPVEEDDIAMYFCQQSRKVPWTFGGGTKVEIK

SEQ ID NO: 200 - protein, 121 aa, synthetic construct
DVQLQESGPGLVKPSQSLSLTCTVTGYSITSDYAWNWIRQFPGNKLEWMGFIIYSGSTNYNPSLESRFSITRDTSKNQFFLQLTSVTTEDTATYYCARSTYGGNYWHFDVWGAGTTVTVSS

SEQ ID NO: 201 - DNA, 39 bp, synthetic construct
  source 1..39: mol_type = other DNA
agtgttgactattatggcacaagtttaatgcagtggtac

SEQ ID NO: 202 - DNA, 24 bp, synthetic construct
  source 1..24: mol_type = other DNA
aacgtagaatctggggtccctgcc

SEQ ID NO: 203 - DNA, 27 bp, synthetic construct
  source 1..27: mol_type = other DNA
caacaaagtaggaaggttccttggacg

SEQ ID NO: 204 - DNA, 33 bp, synthetic construct
  source 1..33: mol_type = other DNA
ggctactcaatcaccagtgattatgcctggaac

SEQ ID NO: 205 - DNA, 45 bp, synthetic construct
  source 1..45: mol_type = other DNA
ataatctacagtggtagcactaactacaacccatctctcgaaagt

SEQ ID NO: 206 - DNA, 42 bp, synthetic construct
  source 1..42: mol_type = other DNA
gccagatcgacctacggtggtaactactggcacttcgatgtc

SEQ ID NO: 207 - DNA, 333 bp, synthetic construct
  source 1..333: mol_type = other DNA
gacattgtgctcacccaatctccagcttctttggctgtgtctctagggcagagagccaccatctcctgcagagccagtgaaagtgttgactattatggcacaagtttaatgcagtggtaccaacagaaaccaggacagccacccaaactcctcatctatgctgcatccaacgtagaatctggggtccctgccaggtttagtggcagtgggtctgggacagacttcagcctcaacatccatcctgtggaggaggatgatattgcaatgtatttctgtcaacaaagtaggaaggttccttggacgttcggtggaggcaccaaggtggaaatcaaa

SEQ ID NO: 208 - DNA, 363 bp, synthetic construct
  source 1..363: mol_type = other DNA
gatgtgcaacttcaggagtcgggacctggcctggttaagccttctcagtctctgtccctcacctgcactgtcactggctactcaatcaccagtgattatgcctggaactggatccggcagtttccaggaaacaaactggagtggatgggcttcataatctacagtggtagcactaactacaacccatctctcgaaagtcgattctctatcactcgagacacatccaagaaccagttcttcctgcaattgacttctgtgactactgaggacacagccacatattactgtgccagatcgacctacggtggtaactactggcacttcgatgtctggggcgcagggaccacggtcaccgtctcctca

SEQ ID NO: 209 - protein, 15 aa, synthetic construct
KASQSVDYDGDSYMN

SEQ ID NO: 210 - protein, 7 aa, synthetic construct
GASNLES

SEQ ID NO: 211 - protein, 9 aa, synthetic construct
QQSIEDPWT

SEQ ID NO: 212 - protein, 6 aa, synthetic construct
GGYNWH

SEQ ID NO: 213 - protein, 16 aa, synthetic construct
DIHKSGSTNYNPSLKS

SEQ ID NO: 214 - protein, 11 aa, synthetic construct
DYYGFSWYFDV

SEQ ID NO: 215 - protein, 111 aa, synthetic construct; identical to SEQ ID NO: 15

SEQ ID NO: 216 - protein, 120 aa, synthetic construct
QVTLKESGPALVKPTQTLTLTCTVTGYSITGGYNWHWIRQPPGKALEWMGDIHKSGSTNYNPSLKSRITISRDTSKNQFVLTMTNMDPVDTATYYCARDYYGFSWYFDVWGQGTTVTVSS

SEQ ID NO: 217 - protein, 15 aa, synthetic construct
KASQSVDYDGDSYMN

SEQ ID NO: 218 - protein, 7 aa, synthetic construct
GASNLES

SEQ ID NO: 219 - protein, 9 aa, synthetic construct
QQSIEDPWT

SEQ ID NO: 220 - protein, 6 aa, synthetic construct
GGYNWH

SEQ ID NO: 221 - protein, 16 aa, synthetic construct
DIHKSGSTNYNPSLKS

SEQ ID NO: 222 - protein, 11 aa, synthetic construct
DYYGFSWYFDV

SEQ ID NO: 223 - protein, 111 aa, synthetic construct; identical to SEQ ID NO: 15

SEQ ID NO: 224 - protein, 120 aa, synthetic construct
QVTLKESGPALVKPTQTLTLTCTVTGYSITGGYNWHWIRQPPGKALEWIGDIHKSGSTNYNPSLKSRITISRDTSKNQFVLTMTNMDPVDTATYYCARDYYGFSWYFDVWGQGTTVTVSS

SEQ ID NO: 225 - protein, 15 aa, synthetic construct
KASQSVDYDGDSYMN

SEQ ID NO: 226 - protein, 7 aa, synthetic construct
GASNLES

SEQ ID NO: 227 - protein, 9 aa, synthetic construct
QQSIEDPWT

SEQ ID NO: 228 - protein, 6 aa, synthetic construct
GGYNWH

SEQ ID NO: 229 - protein, 16 aa, synthetic construct
DIHKSGSTNYNPSLKT

SEQ ID NO: 230 - protein, 11 aa, synthetic construct
DYYGFSWYFDV

SEQ ID NO: 231 - protein, 111 aa, synthetic construct; identical to SEQ ID NO: 15

SEQ ID NO: 232 - protein, 120 aa, synthetic construct; identical to SEQ ID NO: 80

SEQ ID NO: 233 - protein, 15 aa, synthetic construct
KASQSVDYDGDSYMN

SEQ ID NO: 234 - protein, 7 aa, synthetic construct
GASNLES

SEQ ID NO: 235 - protein, 9 aa, synthetic construct
QQSIEDPWT

SEQ ID NO: 236 - protein, 6 aa, synthetic construct
GGYNWH

SEQ ID NO: 237 - protein, 16 aa, synthetic construct
DIHKSGSTNYNPSLKS

SEQ ID NO: 238 - protein, 11 aa, synthetic construct
DYYGFSWYFDV

SEQ ID NO: 239 - protein, 111 aa, synthetic construct; identical to SEQ ID NO: 15

SEQ ID NO: 240 - protein, 120 aa, synthetic construct
QVTLKESGPALVKPTQTLTLTCTVTGYSITGGYNWHWIRQPPGKALEWMGDIHKSGSTNYNPSLKSRLTISRDTSKNQFVLTMTNMDPVDTATYYCARDYYGFSWYFDVWGQGTTVTVSS

SEQ ID NO: 241 - protein, 15 aa, synthetic construct
KASQSVDYDGDSYMN

SEQ ID NO: 242 - protein, 7 aa, synthetic construct
GASNLES

SEQ ID NO: 243 - protein, 9 aa, synthetic construct
QQSIEDPWT

SEQ ID NO: 244 - protein, 6 aa, synthetic construct
GGYNWH

SEQ ID NO: 245 - protein, 16 aa, synthetic construct
DIHKSGSTNYNPSLKS

SEQ ID NO: 246 - protein, 11 aa, synthetic construct
DYYGFSWYFDV

SEQ ID NO: 247 - protein, 111 aa, synthetic construct; identical to SEQ ID NO: 15

SEQ ID NO: 248 - protein, 120 aa, synthetic construct
QVTLKESGPALVKPTQTLTLTCTVTGYSITGGYNWHWIRQPPGKALEWMGDIHKSGSTNYNPSLKSRVTISRDTSKNQFVLTMTNMDPVDTATYYCARDYYGFSWYFDVWGQGTTVTVSS

SEQ ID NO: 249 - protein, 15 aa, synthetic construct
KASQSVDYDGDSYMN

SEQ ID NO: 250 - protein, 7 aa, synthetic construct
GASNLES

SEQ ID NO: 251 - protein, 9 aa, synthetic construct
QQSIEDPWT

SEQ ID NO: 252 - protein, 6 aa, synthetic construct
GGYNWH

SEQ ID NO: 253 - protein, 16 aa, synthetic construct
DIHKSGSTNYNPSLKT

SEQ ID NO: 254 - protein, 11 aa, synthetic construct
DYYGFSWYFDV

SEQ ID NO: 255 - protein, 111 aa, synthetic construct; identical to SEQ ID NO: 15

SEQ ID NO: 256 - protein, 120 aa, synthetic construct
QVTLKESGPALVKPTQTLTLTCTVTGYSITGGYNWHWIRQPPGKALEWIGDIHKSGSTNYNPSLKTRITISRDTSKNQFVLTMTNMDPVDTATYYCARDYYGFSWYFDVWGQGTTVTVSS

SEQ ID NO: 257 - protein, 15 aa, synthetic construct
KASQSVDYDGDSYMN

SEQ ID NO: 258 - protein, 7 aa, synthetic construct
GASNLES

SEQ ID NO: 259 - protein, 9 aa, synthetic construct
QQSIEDPWT

SEQ ID NO: 260 - protein, 6 aa, synthetic construct
GGYNWH

SEQ ID NO: 261 - protein, 16 aa, synthetic construct
DIHKSGSTNYNPSLKT

SEQ ID NO: 262 - protein, 11 aa, synthetic construct
DYYGFSWYFDV

SEQ ID NO: 263 - protein, 111 aa, synthetic construct; identical to SEQ ID NO: 15

SEQ ID NO: 264 - protein, 120 aa, synthetic construct
QVTLKESGPALVKPTQTLTLTCTVTGYSITGGYNWHWIRQPPGKALEWMGDIHKSGSTNYNPSLKTRLTISRDTSKNQFVLTMTNMDPVDTATYYCARDYYGFSWYFDVWGQGTTVTVSS

SEQ ID NO: 265 - protein, 15 aa, synthetic construct
KASQSVDYDGDSYMN

SEQ ID NO: 266 - protein, 7 aa, synthetic construct
GASNLES

SEQ ID NO: 267 - protein, 9 aa, synthetic construct
QQSIEDPWT

SEQ ID NO: 268 - protein, 6 aa, synthetic construct
GGYNWH

SEQ ID NO: 269 - protein, 16 aa, synthetic construct
DIHKSGSTNYNPSLKT

SEQ ID NO: 270 - protein, 11 aa, synthetic construct
DYYGFSWYFDV

SEQ ID NO: 271 - protein, 111 aa, synthetic construct; identical to SEQ ID NO: 15

SEQ ID NO: 272 - protein, 120 aa, synthetic construct
QVTLKESGPALVKPTQTLTLTCTVTGYSITGGYNWHWIRQPPGKALEWMGDIHKSGSTNYNPSLKTRVTISRDTSKNQFVLTMTNMDPVDTATYYCARDYYGFSWYFDVWGQGTTVTVSS

SEQ ID NO: 273 - protein, 15 aa, synthetic construct
KASQSVDYDGDSYMN

SEQ ID NO: 274 - protein, 7 aa, synthetic construct
GASNLES

SEQ ID NO: 275 - protein, 9 aa, synthetic construct
QQSIEDPWT

SEQ ID NO: 276 - protein, 6 aa, synthetic construct
GGYNWH

SEQ ID NO: 277 - protein, 16 aa, synthetic construct
DIHKSGSTNYNPSLKS

SEQ ID NO: 278 - protein, 11 aa, synthetic construct
DYYGFSWYFDV

SEQ ID NO: 279 - protein, 111 aa, synthetic construct; identical to SEQ ID NO: 15

SEQ ID NO: 280 - protein, 120 aa, synthetic construct
QVTLKESGPALVKPTQTLTLTCTVTGYSITGGYNWHWIRQPPGKALEWIGDIHKSGSTNYNPSLKSRLTISRDTSKNQFVLTMTNMDPVDTATYYCARDYYGFSWYFDVWGQGTTVTVSS

SEQ ID NO: 281 - protein, 15 aa, synthetic construct
KASQSVDYDGDSYMN

SEQ ID NO: 282 - protein, 7 aa, synthetic construct
GASNLES

SEQ ID NO: 283 - protein, 9 aa, synthetic construct
QQSIEDPWT

SEQ ID NO: 284 - protein, 6 aa, synthetic construct
GGYNWH

SEQ ID NO: 285 - protein, 16 aa, synthetic construct
DIHKSGSTNYNPSLKS

SEQ ID NO: 286 - protein, 11 aa, synthetic construct
DYYGFSWYFDV

SEQ ID NO: 287 - protein, 111 aa, synthetic construct; identical to SEQ ID NO: 15

SEQ ID NO: 288 - protein, 120 aa, synthetic construct
QVTLKESGPALVKPTQTLTLTCTVTGYSITGGYNWHWIRQPPGKALEWIGDIHKSGSTNYNPSLKSRVTISRDTSKNQFVLTMTNMDPVDTATYYCARDYYGFSWYFDVWGQGTTVTVSS

SEQ ID NO: 289 - protein, 15 aa, synthetic construct
KASQSVDYDGDSYMN

SEQ ID NO: 290 - protein, 7 aa, synthetic construct
GASNLES

SEQ ID NO: 291 - protein, 9 aa, synthetic construct
QQSIEDPWT

SEQ ID NO: 292 - protein, 6 aa, synthetic construct
GGYNWH

SEQ ID NO: 293 - protein, 16 aa, synthetic construct
DIHKSGSTNYNPSLKS

SEQ ID NO: 294 - protein, 11 aa, synthetic construct
DYYGFSWYFDV

SEQ ID NO: 295 - protein, 111 aa, synthetic construct; identical to SEQ ID NO: 15

SEQ ID NO: 296 - protein, 120 aa, synthetic construct; identical to SEQ ID NO: 16

SEQ ID NO: 297 - protein, 15 aa, synthetic construct
KASQSVDYDGDSYMN

SEQ ID NO: 298 - protein, 7 aa, synthetic construct
GASNRES

SEQ ID NO: 299 - protein, 9 aa, synthetic construct
QQSIEDPWT

SEQ ID NO: 300 - protein, 6 aa, synthetic construct
GGYNWH

SEQ ID NO: 301 - protein, 16 aa, synthetic construct
DIHKSGSTNYNPSLKS

SEQ ID NO: 302 - protein, 11 aa, synthetic construct
DYYGFSWYFDV

SEQ ID NO: 303 - protein, 111 aa, synthetic construct; identical to SEQ ID NO: 23

SEQ ID NO: 304 - protein, 120 aa, synthetic construct; identical to SEQ ID NO: 48

SEQ ID NO: 305 - protein, 15 aa, synthetic construct
RASQSVDYDGDSYMN

SEQ ID NO: 306 - protein, 7 aa, synthetic construct
GASNLES

SEQ ID NO: 307 - protein, 9 aa, synthetic construct
QQSIEDPWT

SEQ ID NO: 308 - protein, 6 aa, synthetic construct
GGYNWH

SEQ ID NO: 309 - protein, 16 aa, synthetic construct
DIHKSGSTNYNPSLKT

SEQ ID NO: 310 - protein, 11 aa, synthetic construct
DYYGFSWYFDV

SEQ ID NO: 311 - protein, 111 aa, synthetic construct; identical to SEQ ID NO: 31

SEQ ID NO: 312 - protein, 120 aa, synthetic construct; identical to SEQ ID NO: 80

SEQ ID NO: 313 - protein, 15 aa, synthetic construct
RASQSVDYDGDSYMN

SEQ ID NO: 314 - protein, 7 aa, synthetic construct
GASNRET

SEQ ID NO: 315 - protein, 9 aa, synthetic construct
QQSIEDPWT

SEQ ID NO: 316 - protein, 6 aa, synthetic construct
GGYNWH

SEQ ID NO: 317 - protein, 16 aa, synthetic construct
DIHKSGSTNYNPSLKT

SEQ ID NO: 318 - protein, 11 aa, synthetic construct
DYYGFSWYFDV

SEQ ID NO: 319 - protein, 111 aa, synthetic construct; identical to SEQ ID NO: 39

SEQ ID NO: 320 - protein, 120 aa, synthetic construct; identical to SEQ ID NO: 112

SEQ ID NO: 321 - protein, 15 aa, synthetic construct
KASQSVDYDGDSYMN

SEQ ID NO: 322 - protein, 7 aa, synthetic construct
GASNLES

SEQ ID NO: 323 - protein, 9 aa, synthetic construct
QQSIEDPWT

SEQ ID NO: 324 - protein, 6 aa, synthetic construct
GGYNWH

SEQ ID NO: 325 - protein, 16 aa, synthetic construct
DIHKSGSTNYNPSLKS

SEQ ID NO: 326 - protein, 11 aa, synthetic construct
DYYGFSWYFDV

SEQ ID NO: 327 - protein, 111 aa, synthetic construct
DIVLTQSPASLAVSPGERATITCKASQSVDYDGDSYMNWYQQKPGQPPKLLIYGASNLESGVPDRFSGSGSGRDFTLTISRVEAEDVAVYYCQQSIEDPWTFGQGTKVEIK

SEQ ID NO: 328 - protein, 120 aa, synthetic construct
QVQLQESGPGLVKPSQTLSLTCTVSGYSITGGYNWHWIRQPPGKGLEWMGDIHKSGSTNYNPSLKSRITISRDTSKNQFSLKLSSVTAADTAVYYCARDYYGFSWYFDVWGRGTLVTVSS

SEQ ID NO: 329 - protein, 15 aa, synthetic construct
KASQSVDYDGDSYLA

SEQ ID NO: 330 - protein, 7 aa, synthetic construct
GASNRAS

SEQ ID NO: 331 - protein, 9 aa, synthetic construct
QQSIEDPWT

SEQ ID NO: 332 - protein, 6 aa, synthetic construct
GGYNWH

SEQ ID NO: 333 - protein, 16 aa, synthetic construct
WIHKSGSTNYLPSLQS

SEQ ID NO: 334 - protein, 11 aa, synthetic construct
DYYGFSWYFDV

SEQ ID NO: 335 - protein, 111 aa, synthetic construct
EIVLTQSPATLAVSLGQRATLSCKASQSVDYDGDSYLAWYQQKPGQPPRLLIYGASNRASGIPARFSGSGSGRDFTLNIHGVEEEDFATYYCQQSIEDPWTFGQGTKLEIK

SEQ ID NO: 336 - protein, 120 aa, synthetic construct
DVQLVESGPEAKKPGASLSLTCKVTGYSITGGYNWHWIRQAPGNKLEWMGWIHKSGSTNYLPSLQSRISITRDTSINTFFMELNSVTTEDTAVYYCARDYYGFSWYFDVWGAGTTVTVSS

SEQ ID NO: 337 - protein, 15 aa, synthetic construct
SGDQSVDYDGDSYAH

SEQ ID NO: 338 - protein, 7 aa, synthetic construct
GASKRPS

SEQ ID NO: 339 - protein, 9 aa, synthetic construct
QQSIEDPWT

SEQ ID NO: 340 - protein, 6 aa, synthetic construct
GGYNWH

SEQ ID NO: 341 - protein, 16 aa, synthetic construct
DIHKSGSTNYNPSLKS

SEQ ID NO: 342 - protein, 11 aa, synthetic construct
DYYGFSWYFDV

SEQ ID NO: 343 - protein, 111 aa, synthetic construct
SIELTQPPSSVSVSPGQTARITCSGDQSVDYDGDSYAHWYQQKPGQAPVLLIYGASKRPSGIPERFSGSGSGRTFTLTISGVQAEDEADYYCQQSIEDPWTFGGGTKLTVL

SEQ ID NO: 344 - protein, 120 aa, synthetic construct
QVQLQESGPGLVKPSQTLSLTCTVTGYSITGGYNWHWIRQFPGKKLEWMGDIHKSGSTNYNPSLKSRISITRDTSKNQFFLQLNSVTAEDTATYYCARDYYGFSWYFDVWGAGTTVTVSS

(19) United States
(12) Patent Application Publication
(10) Pub. No.: US 2026/0035466 A1
(43) Pub. Date: Feb. 5, 2026

(54) VISTA-BINDING ANTIBODIES AND METHODS OF USE THEREOF

(71) Applicant: Antharis Therapeutics Inc., San Diego, CA (US)

(72) Inventors: Raphael Ribeiro-Pinaud, San Diego, CA (US); Jinming Xia, San Diego, CA (US); Eric R. Sjoberg, San Diego, CA (US); Seyedehsareh Seyedi, San Diego, CA (US); Jerome Han, San Diego, CA (US); Mary Dwyer, San Diego, CA (US)

(21) Appl. No.: 19/287,514

(22) Filed: Jul. 31, 2025

Related U.S. Application Data
(60) Provisional application No. 63/678,373, filed on Aug. 1, 2024.
(60) Provisional application No. 63/826,302, filed on Jun. 18, 2025.

(51) Int. Cl. C07K 16/28; A61K 39/00; A61P 35/00

(52) U.S. Cl. CPC C07K 16/2827; A61P 35/00; C07K 16/2818; A61K 2039/507; C07K 2317/24; C07K 2317/565; C07K 2317/622

(57) ABSTRACT

Provided herein are, inter alia, novel antibodies that bind to V-domain Ig suppressor of T-cell activation (VISTA) thereby effectively targeting cells expressing VISTA. The antibodies provided herein may be used, inter alia, for therapeutic cancer applications, including, in some embodiments, treatment of multiple cancer types, which may include lung cancer, breast cancer, pancreatic cancer, ovarian cancer, colorectal cancer, renal cancer, or glioblastoma.

### CROSS-REFERENCE TO RELATED APPLICATIONS

This application claims the benefit of priority under 35 U.S.C. § 119(e) to U.S. Provisional Patent Application Ser. No. 63/678,373, filed Aug. 1, 2024 and to U.S. Provisional Patent Application Ser. No. 63,826,302, filed Jun. 18, 2025, which are hereby incorporated by reference in their entireties.

### REFERENCE TO A “SEQUENCE LISTING,” A TABLE, OR A COMPUTER PROGRAM LISTING APPENDIX SUBMITTED ON A COMPACT DISK

The Sequence Listing written in file 057868-503001WO_Sequence_Listing_ST26.xml, created on Jul. 28, 2025, 309,404 bytes, machine format IBM-PC, MS-Windows operating system, is hereby incorporated by reference in its entirety for all purposes.

### BACKGROUND

Enormous quantities of resources have been dedicated to targeting mechanisms of tumor growth and progression, with only limited success. Immunotherapy has emerged as a prominent strategy in cancer treatment, yet despite remarkable improvements, resistance to existing immune checkpoint therapies remains persistent. Oftentimes, technologies showing some benefit in the treatment of tumors and cancers are severely limited with respect to the breadth of mechanisms advantageously affected by the therapy and/or with respect to the variety of tumor and cancer types that show improvement after treatment. In many cases, an anti-tumor technology shows improvement in only one respect and/or shows efficacy in only one tumor type.

### BRIEF SUMMARY

Disclosed herein are an anti-V-domain Ig suppressor of T-Cell Activation (VISTA) Antibody comprising a light chain variable domain and a heavy chain variable domain, wherein said light chain variable domain comprises: a CDR1, a CDR2, and a CDR3 as set forth in Table 5; and wherein said heavy chain variable domain comprises: a CDR1, a CDR2, a CDR3 as set forth in Table 5. In some embodiments, said light chain variable domain comprises an amino acid sequence with at least 80%, 85%, 90%, 91%, 92%, 93%, 94%, 95%, 96%, 97%, 98%, 99%, or 100% sequence identity with the sequence of a light chain variable domain sequence as set forth in Table 5. In some embodiments, said light chain variable domain comprises an amino acid sequence with at least 80%, 85%, 90%, 91%, 92%, 93%, 94%, 95%, 96%, 97%, 98%, 99%, or 100% sequence identity with a sequence selected from SEQ ID NO: 7, SEQ ID NO:15, SEQ ID NO:23, SEQ ID NO:31, SEQ ID NO:39, SEQ ID NO:47, SEQ ID NO: 55, SEQ ID NO: 63, SEQ ID NO:71, SEQ ID NO: 79, SEQ ID NO:87, SEQ ID NO:95, SEQ ID NO: 103, SEQ ID NO: 111, SEQ ID NO: 119, SEQ ID NO: 127, SEQ ID NO: 135. SEQ ID NO: 143. SEQ ID NO: 151, SEQ ID NO: 159, SEQ ID NO: 167, SEQ ID NO: 175, SEQ ID NO:183, SEQ ID NO: 191. SEQ ID NO: 199, SEQ ID NO:207, SEQ ID NO:215, SEQ ID NO:223, SEQ ID NO:231. SEQ ID NO: 239, SEQ ID NO:247, SEQ ID NO:255, SEQ ID NO:263, SEQ ID NO:271, SEQ ID NO:279, SEQ ID NO: 287, SEQ ID NO:295, SEQ ID NO:303, SEQ ID NO:311, SEQ ID NO:319, SEQ ID NO:327, SEQ ID NO:335, or SEQ ID NO:343. In some embodiments, said heavy chain variable domain comprises an amino acid sequence with at least 80%, 85%, 90%, 91%, 92%, 93%, 94%, 95%, 96%, 97%, 98%, 99%, or 100% sequence identity with a heavy chain variable domain sequence as set forth in Table 5. In some embodiments, said heavy chain variable domain comprises an amino acid sequence with at least 80%, 85%, 90%, 91%, 92%, 93%, 94%, 95%, 96%, 97%, 98%, 99%, or 100% sequence identity with a sequence selected from SEQ ID NO:8, SEQ ID NO:16, SEQ ID NO:24, SEQ ID NO:32, SEQ ID NO:40, SEQ ID NO:48, SEQ ID NO:56, SEQ ID NO:64, SEQ ID NO:72, SEQ ID NO:80, SEQ ID NO:88, SEQ ID NO: 96, SEQ ID NO:104, SEQ ID NO:112, SEQ ID NO: 120, SEQ ID NO: 128, SEQ ID NO: 136, SEQ ID NO: 144, SEQ ID NO:152, SEQ ID NO:160, SEQ ID NO:168, SEQ ID NO: 176, SEQ ID NO:184, SEQ ID NO: 192, SEQ ID NO:200, SEQ ID NO:208, SEQ ID NO:216, SEQ ID NO:224, SEQ ID NO: 232, SEQ ID NO:240, SEQ ID NO:248, SEQ ID NO:256, SEQ ID NO:264, SEQ ID NO:272, SEQ ID NO: 280, SEQ ID NO:288, SEQ ID NO:296, SEQ ID NO:304, SEQ ID NO:312, SEQ ID NO:320, SEQ ID NO:328, SEQ ID NO:336, or SEQ ID NO:344. Disclosed herein are an anti-V-domain Ig suppressor of T-Cell Activation (VISTA) antibody comprising a light chain variable domain and a heavy chain variable domain, wherein said light chain variable domain comprises: a CDR1 as set forth in SEQ ID NO: 1, a CDR2 as set forth in SEQ ID NO:2 and a CDR3 as set forth in SEQ ID NO:3; and wherein said heavy chain variable domain comprises: a CDR1 as set forth in SEQ ID NO:4, a CDR2 as set forth in SEQ ID NO:5, and a CDR3 as set forth in SEQ ID NO:6. In some embodiments, said light chain variable domain comprises an amino acid sequence with at least 80%, 85%, 90%, 91%, 92%, 93%, 94%, 95%, 96%, 97%, 98%, 99%, or 100% sequence identity with the sequence of SEQ ID NO:7. In some embodiments, said light chain variable domain comprises an amino acid sequence with at least 80%, 85%, 90%, 91%, 92%, 93%, 94%, 95%, 96%, 97%, 98%, 99%, or 100% sequence identity with the sequence of SEQ ID NO:8. In some embodiments, said antibody is a humanized antibody. In some embodiments, said antibody is a chimeric antibody. In some embodiments, said antibody is an IgG. In some embodiments, said antibody is an IgG1. In some embodiments, said antibody is a Fab′ fragment. In some embodiments, said antibody is a single chain antibody (scFv). In some embodiments, said light chain variable domain and said heavy chain variable domain form part of a scFv. In some embodiments, said antibody is capable of binding VISTA. In some embodiments, said antibody is bound to VISTA. In some embodiments, said VISTA forms part of a cell. In some embodiments, said cell is selected from a lymphoid cell, a myeloid cell, or a stem cell. In some embodiments, said cell is lymphoid cell selected from a B cell or a T cell. In some embodiments, the anti-VISTA antibody binds the same epitope as an antibody comprising: a heavy chain variable region domain comprising a CDR1, a CDR2, and a CDR3 as set forth in Table 5, and a light chain variable domain comprising a CDR1, a CDR2, and a CDR3 as set forth in Table 5. In some embodiments, said light chain variable domain comprises an amino acid sequence with at least 80%, 85%, 90%, 91%, 92%, 93%, 94%, 95%, 96%, 97%, 98%, 99%, or 100% sequence identity with the sequence of a light chain variable domain sequence as set forth in Table 5. In some embodiments, said heavy chain variable domain comprises an amino acid sequence with at least 80%, 85%, 90%, 91%, 92%, 93%, 94%, 95%, 96%, 97%, 98%, 99%, or 100% sequence identity with a heavy chain variable domain sequence as set forth in Table 5. In some embodiments, the anti-VISTA antibody binds the same epitope as an antibody comprising: a heavy chain variable region domain comprising a CDR1 as set forth in SEQ ID NO: 1, a CDR2 as set forth in SEQ ID NO:2, and a CDR3 as set forth in SEQ ID NO:3, and a light chain variable domain comprising a CDR1 as set forth in SEQ ID NO:4, a CDR2 as set forth in SEQ ID NO:5, and a CDR3 as set forth in SEQ ID NO:6. In some embodiments, the light chain variable domain comprises an amino acid sequence with at least 80%, 85%, 90%, 91%, 92%, 93%, 94%, 95%, 96%, 97%, 98%, 99%, or 100% sequence identity with the sequence of SEQ ID NO:7. In some embodiments, the heavy chain variable domain comprises an amino acid sequence with at least 80%, 85%, 90%, 91%, 92%, 93%, 94%, 95%, 96%, 97%, 98%, 99%, or 100% sequence identity with the sequence of SEQ ID NO:8.

In various embodiments, an isolated nucleic acid encodes an antibody described herein. In some embodiments, the isolated nucleic acid comprises a light chain variable domain sequence and a heavy chain variable domain sequence, wherein said light chain variable domain sequence encodes an amino acid sequence comprising: a CDR1, a CDR2, and a CDR3 as set forth in Table 5; or wherein said heavy chain variable domain sequence encodes an amino acid sequence comprising: a CDR1, a CDR2, and a CDR3 as set forth in Table 5. In some embodiments, the isolated nucleic acid comprises a light chain variable domain sequence and a heavy chain variable domain sequence, wherein said light chain variable domain sequence encodes an amino acid sequence comprising: a CDR1, a CDR2, and a CDR3 as set forth in Table 5; or wherein said heavy chain variable domain sequence encodes an amino acid sequence comprising: a CDR1, a CDR2, and a CDR3 as set forth in Table 5. In some embodiments, the isolated nucleic acid comprises a light chain variable domain sequence and a heavy chain variable domain sequence, wherein said light chain variable domain sequence encodes an amino acid sequence comprising: a CDR1, a CDR2, and a CDR3 as set forth in Table 5; and wherein said heavy chain variable domain sequence encodes an amino acid sequence comprising: a CDR1, a CDR2, and a CDR3 as set forth in Table 5. In some embodiments, said light chain variable domain sequence encodes an amino acid sequence with at least 80%, 85%, 90%, 91%, 92%, 93%, 94%, 95%, 96%, 97%, 98%, 99%, or 100% sequence identity with a light chain amino acid sequence as set forth in Table 5. In some embodiments, said heavy chain variable domain encodes an amino acid sequence with at least 80%, 85%, 90%, 91%, 92%, 93%, 94%, 95%, 96%, 97%, 98%, 99%, or 100% sequence identity with a heavy chain amino acid sequence as set forth in Table 5. In various embodiments, a cell comprises an isolated nucleic acid described herein.

In various embodiments, a pharmaceutical composition comprises a therapeutically effective amount of an antibody described herein and a pharmaceutically acceptable excipient. In some embodiments, said pharmaceutical composition is formulated in an aqueous solution at a concentration of 0.200 mg/mL to 2.00 mg/mL, 0.500 mg/mL to 1.50 mg/mL, 0.700 mg/mL to 0.875 mg/mL, or 0.875 mg/mL to 1.25 mg/mL. In some embodiments, said pharmaceutical composition is formulated for intratumoral delivery. In some embodiments, the pharmaceutical composition further comprises a therapeutically effective amount of an antineoplastic agent.

In various embodiments, a method of treating a subject in need thereof comprises administering to a subject a therapeutically effective amount of an antibody described herein. In some embodiments, the method further comprises administering a therapeutically effective amount of an antineoplastic agent. In some embodiments, the effective amount of an antibody and the effective amount of said antineoplastic agent are a combined synergistic amount. In some embodiments, said subject has a tumor or a cancer, is at risk of developing the tumor or the cancer, or is suspected of having said tumor or said cancer. In some embodiments, said cancer is selected from non-small cell lung cancer, breast cancer, ovarian cancer, renal cancer, colorectal cancer, pancreatic cancer, or a glioblastoma. In some embodiments, said antibody is administered to said subject at a concentration of 0.200 mg/mL to 2.00 mg/mL, 0.500 mg/mL to 1.50 mg/mL, 0.700 mg/mL to 0.875 mg/mL, or 0.875 mg/mL to 1.25 mg/mL.

Also disclosed herein are uses of any antibody described herein in the manufacture of a medicament for the treatment of a tumor or cancer.

### BRIEF DESCRIPTION OF THE DRAWINGS

FIG. 1 presents illustrative mean fluorescence intensity (MFI) data for flow cytometry cell surface affinity binding assays using various antibody constructs, in accordance with embodiments.

FIG. 2 presents illustrative data for enzyme-linked immunosorbent assay (ELISA) binding data for antibody constructs to human VISTA protein, in accordance with embodiments.

FIG. 3 presents illustrative data for ELISA binding assay data for antibody constructs to Cynomolgus-VISTA, in accordance with embodiments.

FIG. 4 presents illustrative data for antibody construct performance in VISTA-VSIG3 inhibition assays, in accordance with embodiments.

FIG. 5 presents illustrative data of cytokine release during VISTA-VSIG3 blocking assays, in accordance with embodiments.

FIG. 6 presents illustrative data for interferon-gamma (IFN-γ) secretion by human allogeneic mixed lymphocytes in response to antibody construct treatment, in accordance with embodiments.

FIG. 7A presents illustrative cell surface binding data for anti-VISTA antibodies, in accordance with embodiments.

FIG. 7B presents illustrative data showing VISTA-to-VSIG3 binding blockade using anti-VISTA antibodies described herein, in accordance with embodiments.

FIG. 8 presents illustrative data showing interferon gamma (IFN-γ) secretion by peripheral blood mononuclear cells (PBMC) in the presence of anti-VISTA antibodies described herein, in accordance with embodiments.

FIG. 9 presents illustrative data showing tumor necrosis factor (TNF-α) secretion by peripheral blood mononuclear cells (PBMC) in the presence of anti-VISTA antibodies described herein, in accordance with embodiments.

FIG. 10 presents illustrative data showing interferon gamma (IFN-γ) secretion by peripheral blood mononuclear cells (PBMC) in the presence of anti-VISTA antibodies described herein with anti-CD3 co-treatment, in accordance with embodiments.

FIG. 11A shows a graphical representation of an in vivo treatment regimen used to test AT-05 family anti-VISTA antibodies, in accordance with embodiments. FIG. 11B presents a study design for testing efficacy of AT-05 family anti-VISTA antibodies, in accordance with embodiments.

FIG. 12A presents illustrative data showing tumor necrosis factor (TNF-α) secretion by peripheral blood mononuclear cells (PBMC) in the presence of anti-VISTA antibodies described herein, in accordance with embodiments.

FIG. 12B presents illustrative data showing tumor necrosis factor (TNF-α) secretion by peripheral blood mononuclear cells (PBMC) in the presence of anti-VISTA antibodies described herein, in accordance with embodiments.

FIG. 12C presents illustrative data showing tumor necrosis factor (TNF-α) secretion by peripheral blood mononuclear cells (PBMC) in the presence of anti-VISTA antibodies described herein, in accordance with embodiments.

FIG. 13A presents illustrative data showing tumor necrosis factor (TNF-α) secretion by peripheral blood mononuclear cells (PBMC) in the presence of anti-VISTA antibodies described herein, in accordance with embodiments.

FIG. 13B presents illustrative data showing tumor necrosis factor (TNF-α) secretion by peripheral blood mononuclear cells (PBMC) in the presence of anti-VISTA antibodies described herein, in accordance with embodiments.

FIG. 13C presents illustrative data showing tumor necrosis factor (TNF-α) secretion by peripheral blood mononuclear cells (PBMC) in the presence of anti-VISTA antibodies described herein, in accordance with embodiments.

FIG. 14 presents illustrative data showing tumor necrosis factor (TNF-α) secretion by peripheral blood mononuclear cells (PBMC) in the presence of anti-VISTA antibodies described herein, in accordance with embodiments.

FIG. 15A presents illustrative data showing in vivo tumor volume growth over time in animals treated with an AT05 family anti-VISTA antibody and an anti-PD1 antibody or anti-PD1 antibody alone, in accordance with embodiments.

FIG. 15B presents illustrative data showing in vivo tumor growth inhibition following co-treatment with an AT05 family anti-VISTA antibody and an anti-PD1 antibody, in accordance with embodiments.

FIGS. 16A-16E present illustrative data showing effects of antibody constructs on target cell adhesion, in accordance with embodiments.

FIGS. 16F-16K present illustrative data showing effects of antibody constructs on target cell adhesion relative to control treatment, in accordance with embodiments.

FIG. 16L presents illustrative data showing effects of antibody constructs on blood mononuclear cell viability relative to control treatment, in accordance with embodiments.

FIGS. 16M-16N present illustrative data showing inhibition of migration in target cells by antibody constructs relative to control treatment, in accordance with embodiments.

FIG. 16-O presents illustrative data showing effects of antibody constructs on IL-6 secretion by target cells relative to control treatment, in accordance with embodiments.

FIG. 16P presents illustrative data showing inhibition of angiogenic behavior in human umbilical vein endothelial cells (HUVECs) by antibody constructs relative to control treatment, in accordance with embodiments.

### DETAILED DESCRIPTION

Proliferation and migration are two mechanisms targeted for the treatment of various tumors and cancers; however, inhibition in tumor secretion of inflammatory agents and/or inhibition of tumor cell adhesion are other potential mechanisms for inhibiting tumor growth. Ideally, a single treatment would simultaneously inhibit tumor growth through multiple pathways, but this can be difficult to achieve in practice. An ideal treatment would also be efficacious against multiple types of primary tumors, such as non-small cell lung cancer (NSCLC), breast cancer, pancreatic cancer, breast cancer, colorectal cancer, ovarian cancer, renal cancer, and brain tumors, such as glioblastoma, for instance, to be useful in a broader range of patients having a single primary tumor type and to treat a patient having multiple different primary tumors. Despite decades of research and intense investment in potential technologies, advantageous cross-tumor efficacy has proven difficult to realize. The compositions and methods provided herein address these and other needs in the art.

Anti-VISTA therapies hold great potential for treating various conditions, for example, through suppression of proinflammatory antitumor responses. Consequently, antibodies constructs and therapies described herein, which can affect VISTA (V-domain Ig suppressor of T-cell activation) signaling pathways, can succeed where current immune checkpoint therapies have failed. Accordingly, blockade of VISTA using antibody constructs presented herein represents a valuable avenue for treatment of mammalian cancers.

Acquired resistance to anti-PD1 inhibitors is a prevalent occurrence, which can be accompanied by heightened intra-tumoral VISTA expression, as evidenced by biopsies from affected patients. VISTA blockade can be used to improve clinical outcomes in patients. Anti-VISTA therapy holds promise for various patient cohorts, including those suffering pancreatic cancer, metastatic melanoma, and acquired resistance to PD-1 (programmed cell death protein 1) and CTLA-4 (cytotoxic T-lymphocyte associated protein 4) inhibitor therapies. Elevated VISTA expression levels have been observed in glioma patients, which can correlate to poorer prognosis and diminished survival rates.

VISTA (also known as PD-1H or programmed death-1 homolog) is an immune regulatory molecule with potential for therapeutic intervention. VISTA plays a crucial role in regulating T cell function and maintaining peripheral T cell tolerance.

MDSCs (myeloid derived suppressor cells) of cancer patients can exhibit elevated VISTA expression levels, with VISTA upregulation correlating with acquired resistance to anti-CTLA-4 and PD-1/PD-L1 therapies across various cancer types, including prostate cancer, melanoma, and myeloid leukemia.

The pivotal functional domain of VISTA, which can be essential for inhibiting its binding with VSIG3 (V-Set and Immunoglobulin domain containing 3) and subsequently promoting the suppression of antitumor immune responses can be targeted by antibody constructs described herein (e.g., AT05 asset family antibodies). The AT05 antibody constructs described herein were selected for their high-affinity cell surface binding capabilities and were subjected to a series of rigorous functional assays.

AT05 asset family antibody constructs described herein can act as immune checkpoint inhibitors, which can result in T cell activation, for example, through blocking VISTA-VSIG3 interactions and attenuation of VSIG3-mediated suppression of TNF a (tumor necrosis factor-alpha) and IFN-γ (interferon gamma) release from activated T cells. AT05 asset family antibody constructs can have an IgG4 framework, which can offer advantages including independence of effector function and reduced toxicity.

In some embodiments, AT05 antibody constructs can block VISTA's inhibitory role without resorting to Fc-dependent mechanisms, unlike previous antibodies that utilized IgG1 Fc isotypes. In some embodiments, AT05 anti-VISTA antibody constructs, which can include an IgG4 framework, can inhibit VISTA function without triggering cell depletion, e.g., through Fc-mediated mechanisms.

As shown herein, AT05 family anti-VISTA antibody constructs described herein, which can be chimeric, can enhance binding affinity to monocytes and can increase T cell activation, e.g., via blockade of VISTA interaction to its ligands. In addition, AT05 antibodies can have specific binding between VISTA and both its ligands, VSIG3 and PSGL-1 (P-selectin glycoprotein ligand-1). As shown herein, AT05 family antibody constructs can exhibit enhanced binding affinity to monocytes and increased T cell activation.

AT05 family anti-VISTA antibody constructs described herein, which can comprise a nucleic acid or amino acid residue sequence of any one of SEQ ID NOs. 1 to 208 (one or more sequences shown in Table 5), can be useful in treating a subject having, diagnosed as having, or suspected as having a tumor or cancer. For example, AT05 family anti-VISTA antibody constructs described herein can be useful in treating a solid tumor.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide.” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples of nucleosides include cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region). Some embodiments describe 80%, 85%, 90%, 91%, 92%, 93%, 94%, 95%, 96%, 97%, 98%, 99%, or 100% sequence identity. Such descriptions may include 80%, 85%, 90%, 95%, 98%, 99%, or 100% sequence identity.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may, in embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., VISTA) in other proteins with different numbering systems. For example, by performing a simple sequence alignment with a protein (e.g., VISTA) the identity and location of residues corresponding to specific positions of the protein are identified in other protein sequences aligning to the protein. For example, a selected residue in a selected antibody (or Fab domain) corresponds to light chain threonine at Kabat position 40, when the selected residue occupies the same essential spatial or other structural relationship as a light chain threonine at Kabat position 40. In some embodiments, where a selected protein is aligned for maximum homology with the light chain of an antibody (or Fab domain), the position in the aligned selected protein aligning with threonine 40 is said to correspond to threonine 40. Instead of a primary sequence alignment, a three dimensional structural alignment can also be used, e.g., where the structure of the selected protein is aligned for maximum correspondence with the light chain threonine at Kabat position 40, and the overall structures compared. In this case, an amino acid that occupies the same essential position as threonine 40 in the structural model is said to correspond to the threonine 40 residue.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
(see, e.g., Creighton, Proteins (1984)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2: 482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W. T. and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region, involved in binding the target antigen, and a constant (“C”) region that interacts with other components of the immune system. The light and heavy chain variable regions (also referred to herein as light chain variable (VL) domain and heavy chain variable (VH) domain, respectively) come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable heavy chain of an antibody provided herein includes a CDR1, a CDR2 and a CDR3 (e.g., a heavy chain CDR1 as set forth in Table 5, a heavy chain CDR2 as set forth in Table 5, and a heavy chain CDR3 as set forth in Table 5). In embodiments, the variable light chain of an antibody provided herein includes a CDR1, a CDR2 and a CDR3 (e.g., a light chain CDR1 as set forth in Table 5, a light chain CDR2 as set forth in Table 5, and a light chain CDR3 as set forth in Table 5). See, for example the tables as provided herein. In embodiments, the variable heavy chain provided herein includes in N-terminal to C-terminal direction a CDR H1, a CDR H2 and a CDR H3. In embodiments, the CDRs of the light chain are referred to as CDR1, CDR2, and CDR3 of VL and the CDRs of the heavy chain are referred to as CDR1, CDR2, and CDR3 of VH. See, for example the tables as provided herein.

The terms “FR L1”, “FR L2”, “FR L3” and “FR L4” as provided herein are used according to their common meaning in the art and refer to the framework regions (FR) 1, 2, 3 and 4 of the variable light (L) chain of an antibody. In embodiments, the variable light chain provided herein includes in N-terminal to C-terminal direction a FR L1, a FR L2, a FR L3 and a FR L4. Likewise, the terms “FR H1”, “FR H2”, “FR H3” and “FR H4” as provided herein are used according to their common meaning in the art and refer to the framework regions (FR) 1, 2, 3 and 4 of the variable heavy (H) chain of an antibody. In embodiments, the variable heavy chain provided herein includes in N-terminal to C-terminal direction a FR H1, a FR H2, a FR H3 and a FR H4.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL), variable light chain (VL) domain or light chain variable region and variable heavy chain (VH), variable heavy chain (VH) domain or heavy chain variable region refer to these light and heavy chain regions, respectively. The terms variable light chain (VL), variable light chain (VL) domain and light chain variable region as referred to herein may be used interchangeably. The terms variable heavy chain (VH), variable heavy chain (VH) domain and heavy chain variable region as referred to herein may be used interchangeably. The Fc (i.e. fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins. The term “light chain” is used according to its ordinary meaning in the biological arts, and refers to the polypeptide formed by a light chain variable domain (VL) and a light chain constant domain (CL). Likewise, the term “heavy chain” is used according to its ordinary meaning in the biological arts, and refers to the polypeptide formed by a heavy chain variable domain (VH) and one or more heavy chain constant domains (CH1, CH2, CH3).

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (sec. e.g., McCafferty et al., Nature 348:552-554 (1990)). The term “antibody” as referred to herein further includes antibody variants such as single domain antibodies. Thus, in embodiments an antibody includes a single monomeric variable antibody domain. Thus, in embodiments, the antibody, includes a variable light chain (VL) domain or a variable heavy chain (VH) domain. In embodiments, the antibody is a variable light chain (VL) domain or a variable heavy chain (VH) domain. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

For preparation of monoclonal or polyclonal antibodies, any technique known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy (1985)). “Monoclonal” antibodies (mAb) refer to antibodies derived from a single clone. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A single-chain variable fragment (scFv) is typically a fusion protein of the variable domains of the heavy (VH) and light chain (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31 (3): 169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (e.g. variable region including domain VH and VL) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

Techniques for conjugating therapeutic agents to antibodies are well known (see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)). As used herein, the term “antibody-drug conjugate” or “ADC” refers to a therapeutic agent conjugated or otherwise covalently bound to an antibody.

A “therapeutic agent” as referred to herein, is a composition useful in treating or preventing a disease such as cancer (e.g., leukemia). In embodiments, the therapeutic agent is an anti-cancer agent. “Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer. In some cases, a therapeutic agent can be or can comprise a PD-1 pathway inhibitor. In some embodiments a therapeutic agent can be or can include pembrolizumab (Keytruda®) or a derivative thereof, nivolumab (Opdivo®) or a derivative thereof, cemiplimab (Libtayo®) or a derivative thereof, tislelizumab (Tevimbra®) or a derivative thereof, atezolizumab (TECENTRIQ®) or a derivative thereof, durvalumab (IMFINZI®) or a derivative thereof, avelumab (BAVENCIO®) or a derivative thereof, dostarlimab (JEMPERLI®) or a derivative thereof, retifanlimab (Zynyz®) or a derivative thereof, toripalimab (Loqtorzi®) or a derivative thereof (e.g., wherein the therapeutic agent is a composition comprising an AT-05 antibody described herein).

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a receptor or antibody, antibody variant, antibody region or fragment thereof.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

The term “VISTA” or “VSIR” or “V-set immunoregulatory receptor” or “PD-1H” or “V-type immunoglobulin domain-containing suppressor of T-cell activation” as used herein refers to any recombinant or naturally-occurring forms of V-domain Ig suppressor of T-cell Activation (VISTA) or variants or homologs thereof that maintain VISTA activity (e.g. within at least 50%, 80%, 90%, 95%, 96%, 97%, 98%, 99% or 100% activity compared to VISTA). In embodiments, the variants or homologs have at least 90%, 95%, 96%, 97%, 98%, 99% or 100% amino acid sequence identity across the whole sequence or a portion of the sequence (e.g. a 10, 20, 50, 100, 150 or 200 continuous amino acid portion) compared to a naturally occurring VISTA polypeptide. In embodiments, VISTA is substantially identical to the protein identified by the UniProt reference number Q9H7M9 or a variant or homolog having substantial identity thereto. In embodiments, VISTA is the protein identified by the UniProt reference number Q9H7M9 or a variant or homolog having substantial identity thereto. UniProt numbers reflect the relevant entry at www.uniprot.org as of their effective filing date.

V-domain Ig suppressor of T-cell Activation, also known as VISTA, VSIR, V-set immunoregulatory receptor, and PD-1H, is an immunoregulatory receptor capable of affecting T-cell activation and/or negatively regulating cytokine production. Alterations in VISTA activity have been implicated in certain cancers.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. Expression of a gene from a plasmid can occur in cis or in trans. If a gene is expressed in cis, the gene and the regulatory elements are encoded by the same plasmid. Expression in trans refers to the instance where the gene and the regulatory elements are encoded by separate plasmids.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any appropriate method known in the art for conjugating an antibody to the label may be employed, e.g., using methods described in Hermanson, Bioconjugate Techniques 1996, Academic Press, Inc., San Diego.

When the label or detectable moiety is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups. The chelate is normally linked to the PSMA antibody or functional antibody fragment by a group, which enables the formation of a bond to the molecule with minimal loss of immunoreactivity and minimal aggregation and/or internal cross-linking. The same chelates, when complexed with non-radioactive metals, such as manganese, iron and gadolinium are useful for MRI, when used along with the antibodies and carriers described herein. Macrocyclic chelates such as NOTA, DOTA, and TETA are of use with a variety of metals and radiometals including, but not limited to, radionuclides of gallium, yttrium and copper, respectively. Other ring-type chelates such as macrocyclic polyethers, which are of interest for stably binding nuclides, such as ²²³Ra for RAIT may be used. In certain embodiments, chelating moieties may be used to attach a PET imaging agent, such as an Al-¹⁸F complex, to a targeting molecule for use in PET analysis.

“Contacting” is used in accordance with its plain ordinary meaning and refers to the process of allowing at least two distinct species (e.g. antibodies and antigens) to become sufficiently proximal to react, interact, or physically touch. It should be appreciated; however, that the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be, for example, a pharmaceutical composition as provided herein and a cell. In embodiments contacting includes, for example, allowing a pharmaceutical composition as described herein to interact with a cell.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include, but are not limited to, yeast cells and cells derived from plants and animals, for example mammalian, insect (e.g., spodoptera) and human cells.

A “stem cell” as provided herein refers to a cell characterized by the ability of self-renewal through mitotic cell division and the potential to differentiate into a tissue or an organ. Among mammalian stem cells, embryonic stem cells (ES cells) and somatic stem cells (e.g., hematopoietic stem cells (HSC)) can be distinguished. Embryonic stem cells reside in the blastocyst and give rise to embryonic tissues, whereas somatic stem cells reside in adult tissues for the purpose of tissue regeneration and repair.

“B Cells” or “B lymphocytes” refer to their standard use in the art. B cells are lymphocytes, a type of white blood cell (leukocyte), that develops into a plasma cell (a “mature B cell”), which produces antibodies. An “immature B cell” is a cell that can develop into a mature B cell. Generally, pro-B cells undergo immunoglobulin heavy chain rearrangement to become pro B pre B cells, and further undergo immunoglobulin light chain rearrangement to become an immature B cells. Immature B cells include T1 and T2 B cells.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

As defined herein, the term “inhibition”, “inhibit”, “inhibiting” and the like in reference to cell proliferation (e.g., cancer cell proliferation) means negatively affecting (e.g., decreasing proliferation) or killing the cell. In some embodiments, inhibition refers to reduction of a disease or symptoms of disease (e.g., cancer, cancer cell proliferation). Thus, inhibition includes, at least in part, partially or totally blocking stimulation, decreasing, preventing, or delaying activation, or inactivating, desensitizing, or down-regulating signal transduction or enzymatic activity or the amount of a protein. Similarly, an “inhibitor” is a compound or protein that inhibits a receptor or another protein, e.g., by binding, partially or totally blocking, decreasing, preventing, delaying, inactivating, desensitizing, or down-regulating activity (e.g., a receptor activity or a protein activity).

As defined herein, the term “inhibition”, “inhibit”, “inhibiting” and the like in reference to a protein-inhibitor interaction means negatively affecting (e.g. decreasing) the activity or function of the protein (e.g. VISTA) relative to the activity or function of the protein in the absence of the inhibitor. In embodiments inhibition means negatively affecting (e.g. decreasing) the concentration or levels of a protein (e.g. VISTA) relative to the concentration or level of the protein in the absence of the inhibitor. In embodiments inhibition refers to reduction of a disease or symptoms of disease. In embodiments, inhibition refers to a reduction in the activity of a protein (e.g. VISTA). Thus, inhibition includes, at least in part, partially or totally blocking stimulation, decreasing, preventing, or delaying activation, or inactivating, desensitizing, or down-regulating signal transduction or enzymatic activity or the amount of a protein (e.g. VISTA). In embodiments, inhibition refers to a reduction of activity of a protein (e.g. VISTA) resulting from a direct interaction (e.g. an inhibitor binds to the protein). In embodiments, inhibition refers to a reduction of activity of a protein (e.g. VISTA) from an indirect interaction (e.g. an inhibitor binds to a protein that activates the protein, thereby preventing target protein activation).

Thus, the terms “inhibitor,” “repressor” or “antagonist” or “downregulator” interchangeably refer to a substance capable of detectably decreasing the expression or activity of a given gene or protein (e.g. VISTA). The antagonist can decrease expression or activity of a protein (e.g. VISTA) by 10%, 20%, 30%, 40%, 50%, 60%, 70%, 80%, 90% or more in comparison to a control in the absence of the antagonist. In certain instances, a protein's (e.g. VISTA) expression or activity is 1.5-fold. 2-fold, 3-fold, 4-fold, 5-fold, 10-fold or lower than the expression or activity in the absence of the antagonist.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “recombinant” when used with reference, e.g., to a cell, nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “control” or “standard control” refers to a sample, measurement, or value that serves as a reference, usually a known reference, for comparison to a test sample, measurement, or value. For example, a test sample can be taken from a patient suspected of having a given disease (e.g. cancer) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. For example, a control can be devised to compare therapeutic benefit based on pharmacological data (e.g., half-life) or therapeutic measures (e.g., comparison of side effects). Controls are also valuable for determining the significance of data. For example, if values for a given parameter are widely variant in controls, variation in test samples will not be considered as significant. One of skill will recognize that standard controls can be designed for assessment of any number of parameters (e.g. RNA levels, protein levels, specific cell types, specific bodily fluids, specific tissues, synoviocytes, synovial fluid, synovial tissue, fibroblast-like synoviocytes, macrophage like synoviocytes, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with the compounds or methods provided herein. The disease may be a cancer. In some further instances, “cancer” refers to human cancers and carcinomas, sarcomas, adenocarcinomas, lymphomas, leukemias, including solid and lymphoid cancers, kidney cancer (e.g., renal cell carcinoma), breast cancer (e.g., triple negative breast cancer), lung cancer (e.g., non-small cell lung cancer, small cell lung cancer, or malignant pleural mesothelioma), bladder cancer, colon cancer, colorectal cancer (CRC), ovarian cancer, prostate cancer, pancreatic cancer, biliary tract cancer, gastric cancer, brain cancer, head and neck squamous cell carcinoma, skin cancer (e.g., melanoma, cutaneous squamous cell carcinoma, or merkel cell carcinoma), uterine, cervical cancer, testicular cancer, urothelial cancer, glioma, esophageal cancer, and liver cancer (e.g., hepatocellular carcinoma), including hepatocarcinoma, lymphoma, including B-acute lymphoblastic lymphoma, non-Hodgkin's lymphomas (e.g., Burkitt's, Small Cell, and Large Cell lymphomas), Hodgkin's lymphoma (e.g., classical Hodgkin's lymphoma), leukemia (including acute myeloid leukemia (AML), ALL, and CML), or multiple myeloma.

As used herein, the term “cancer” refers to all types of cancer, neoplasm or malignant tumors found in mammals (e.g., humans), including leukemia, carcinomas and sarcomas. Exemplary cancers that may be treated with a compound or method provided herein include breast cancer, colon cancer, colorectal cancer, kidney cancer, leukemia, lung cancer (e.g., non-small cell lung cancer), melanoma, ovarian cancer, prostate cancer, renal cancer, pancreatic cancer, brain cancer (e.g., glioblastomas and/or astrocytomas), liver cancer, gastric cancer or a sarcoma. In particular, compositions comprising antibodies described herein can be effective at treating a cancer (e.g., colorectal cancer), reducing the risk of a cancer (e.g., colorectal cancer) progressing to a more severe stage (e.g., from Stage 0 to Stage I, Stage II, Stage III, or Stage IV; from Stage I to Stage II, Stage III, or Stage IV; from Stage II to Stage III or Stage IV; or from Stage III to Stage IV), reducing the risk of death of a patient having or suspected of having a cancer (e.g., colorectal cancer), or causing a cancer (e.g., colorectal cancer) to enter remission (e.g., wherein an objective or subjective metric of cancer severity, such as cancer progression, is reduced or eliminated).

The term “colorectal cancer” is taken to mean a tumor arising from the colon or rectum. Colorectal cancers that may be treated with a compound or method provided herein include, for example, colon cancer, rectal cancer, adenocarcinoma, carcinoid tumor, gastrointestinal stromal tumors (GISTs), or lymphomas.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute myeloid leukemia, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma. Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. In some cases, cell migration (e.g., increased cell migration relative to a control cell) can be indicative of a risk of metastasis or a tendency to metastasize in a tumor tissue or can potentiate metastasis, cancer tissue, tumor cell, or cancer cell. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g., cancer (e.g. leukemia, acute myeloid leukemia)) means that the disease (e.g., cancer (e.g. leukemia, acute myeloid leukemia)) is caused by (in whole or in part), or a symptom of the disease is caused by (in whole or in part) the substance or substance activity or function. Alternatively, the substance (e.g., VISTA) may be an indicator of the disease (e.g., cancer (e.g. leukemia, acute myeloid leukemia)). Thus, an associated substance may serve as a means of targeting disease tissue (e.g., cancer cells (e.g., leukemia stem cells, acute myeloid leukemia cells)).

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

A “therapeutic agent” as referred to herein, is a composition useful in treating or preventing a disease such as cancer (e.g., leukemia). In embodiments, the therapeutic agent is an anti-cancer agent. “Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus, in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (Sec, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

A “synergistic amount” as used herein refers to the sum of a first amount (e.g., an amount of a compound provided herein) and a second amount (e.g., a therapeutic agent) that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of the compound administered in combination where the measured effect is greater than the sum of the individual effects of each of the compounds provided herein administered alone as a single agent.

### Antibody Compositions

Provided herein are, inter alia, antibodies (e.g., humanized antibodies, monoclonal antibodies) and antibody compositions (e.g., scFvs, bispecific antibodies) that are capable of binding VISTA. The antibodies and antibody compositions provided herein include novel light and heavy chain domain CDRs and framework regions, and bind VISTA with high efficiency and specificity, thereby effectively targeting these target proteins and/or cells expressing the target proteins. The light and heavy chain domains of the antibodies provided herein may form part of recombinant proteins also referred to herein as antibody compositions (e.g., IgG (for instance, IgG1), scFv, or bispecific antibodies) to be used, inter alia, as cancer therapeutics and for diagnostic purposes.

Anti-VISTA antibody constructs disclosed herein can comprise novel molecular sequences selected from among SEQ ID NO: 1 through SEQ ID NO: 80. Antibodies and antibody fragments (as well as nucleic acid vectors encoding such antibodies and antibody fragments) comprising one or more of these sequences are contemplated herein. Collectively, such antibody constructs and nucleic acid vectors are referred to as the “AT05” or “AT-05” series of assets. For example, “AT-05.01” or “AT05.01” antibodies, antibody fragments, or vectors can comprise one or more molecular sequences selected from among SEQ ID NO:1 through SEQ ID NO:8. In some cases, AT-05.00 antibodies can be mouse anti-VISTA antibodies. Among the AT-05 series assets contemplated herein are AT-05.01. AT-05.01v1. AT-05.01v2, AT-05.01v3, AT-05.01v4, AT-05.01v5, AT-05.01v6, AT-05.01v7, AT-05.01v8, AT-05.01v9. AT-05.01v10, AT-05.01v11, AT-05.01v12, AT-05.01v13, AT-05.01v14, AT-05.01v15, AT-05.01v15v1. AT-05.01v15v2, AT-05.01v15v3, AT-05.01v15v4, AT-05.01v15v5, AT-05.01v15v6, AT-05.01v15v7, AT-05.01v15v8, AT-05.01v15v9, AT-05.01v15v10, AT-05.01v15v11, AT-05.01v15v12, AT-05.01v15v13, AT-05.01v15v14, AT-05.01v15v15, AT-05.01v15v16, AT-05.01v15v17, AT-05.01v16, AT-05.02, AT-05.03, AT-05.04, AT-05.05, AT-05.06, AT-05.07, AT-05.08, or AT-05.00 antibodies and antibody fragments (e.g., chimeric antibodies and antibody fragments), as well as nucleic acid vectors encoding such antibodies and antibody fragments.

In some cases, an anti-VISTA antibody disclosed herein can include a light chain variable domain and a heavy chain variable domain, wherein the light chain variable domain includes: a CDR L1 as set forth in SEQ ID NO:1, a CDR L2 as set forth in SEQ ID NO:2 and a CDR L3 as set forth in SEQ ID NO:3; and wherein the heavy chain variable domain includes: a CDR H1 as set forth in SEQ ID NO: 4, a CDR H2 as set forth in SEQ ID NO:5, and a CDR H3 as set forth in SEQ ID NO:6. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:7. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:8. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 15. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 16. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:23. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 24. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:31. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 32. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:39. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 40. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:47. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 48. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:55. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 56. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:63. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 64. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:71. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 72. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:79. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 80. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:87. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 88. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:95. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 96. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 103. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 104. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 111. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 112. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 119. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 120. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 127. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 128. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 135. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 136. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 143. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 144. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 151. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 152. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 159. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 160. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 167. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 168. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:175. In some cases, an anti-VISTA antibody can include a heavy chain variable domain having an amino acid sequence with at least 80%, 85%, 90%. 91%, 92%, 93%, 94%, 95%, 96%, 97%, 98%, 99%, or 100% sequence identity with the sequence of SEQ ID NO: 176. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:183. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 184. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 191. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 192. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:199. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 200. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:207. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 208. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:215. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 216. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:223. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 224. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:231. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 232. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:239. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 240. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:247. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 248. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:255. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 256. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:263. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 264. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:271. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 272. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:279. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 280. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:287. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 288. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:295. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 296. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:303. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 304. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:311. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 312. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:319. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 320. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:327. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 328. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:335. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 336. In some cases, an anti-VISTA antibody can include a light chain variable domain having an amino acid sequence with at least 80%, 85%, 90%, 91%, 92%, 93%, 94%, 95%, 96%, 97%, 98%, 99%, or 100% sequence identity with the sequence of SEQ ID NO:343. In some cases, an anti-VISTA antibody can include a heavy chain variable domain having an amino acid sequence with at least 80%, 85%, 90%, 91%, 92%, 93%, 94%, 95%, 96%, 97%, 98%, 99%, or 100% sequence identity with the sequence of SEQ ID NO: 344. In some cases, association (e.g., binding) of an anti-VISTA antibody described herein with at least a portion of a VISTA protein can inhibit the adhesion, proliferation, survival, migration, spread (e.g., metastasis), or growth (e.g., diameter) of one or more types of tumor cells, tumor tissues, cancer cells, or cancer tissues (e.g., breast cancer, pancreatic cancer, ovarian cancer, renal cancer, colorectal cancer, brain cancer such as a glioblastoma or astrocytoma, or lung cancer such as a non-small cell lung cancer) in an in vitro or in vivo context. In some cases, an anti-VISTA antibody disclosed herein can be effective at inhibiting one or more of adhesion, proliferation, survival, migration, spread (e.g., metastasis), or growth (e.g., diameter) of one or more tumor cells, tumor tissues, cancer cells, or cancer tissues (e.g., wherein the one or more cells or tissues comprises two or more tumor or cancer types, for example, selected from breast cancer, pancreatic cancer, ovarian cancer, renal cancer, colorectal cancer, brain cancer such as a glioblastoma or astrocytoma, or lung cancer such as a non-small cell lung cancer). In some cases, an anti-VISTA antibody disclosed herein can be effective at increasing or maintaining viability of peripheral mononuclear blood cell(s), e.g., in the context of at least one (e.g., one, two, three, four, or more than four) in vivo or in vitro tumor or cancer cell/tissue types described herein.

As described herein, a “light chain variable (VL) domain” as provided herein refers to the variable region of the light chain of an antibody, an antibody variant or fragment thereof. Likewise, the “heavy chain variable (VH) domain” as provided herein refers to the variable region of the heavy chain of an antibody, an antibody variant or fragment thereof. The light chain variable domain and the heavy chain variable domain together form the paratope, which binds an antigen (epitope). The paratope or antigen-binding site is formed at the N-terminus of an antibody, an antibody variant or fragment thereof. In embodiments, the light chain variable (VL) domain includes CDR L1, CDR L2, CDR L3 and FR L1. FR L2, FR L3 and FR L4 (framework regions) of an antibody light chain. In embodiments, the heavy chain variable (VH) domain includes CDR H1, CDR H2, CDR H3 and FR H1, FR H2, FR H3 and FR H4 (framework regions) of an antibody heavy chain. In embodiments, the light chain variable (VL) domain and a light chain constant (CL) domain form part of an antibody light chain. In embodiments, the heavy chain variable (VH) domain and a heavy chain constant (CH1) domain form part of an antibody heavy chain. In embodiments, the heavy chain variable (VH) domain and one or more heavy chain constant (CH1, CH2, or CH3) domains form part of an antibody heavy chain. Thus, in embodiments, the light chain variable (VL) domain forms part of an antibody. In embodiments, the heavy chain variable (VH) domain forms part of an antibody. In embodiments, the light chain variable (VL) domain forms part of a therapeutic antibody. In embodiments, the heavy chain variable (VH) domain forms part of a therapeutic antibody. In embodiments, the light chain variable (VL) domain forms part of a human antibody. In embodiments, the heavy chain variable (VH) domain forms part of a human antibody. In embodiments, the light chain variable (VL) domain forms part of a humanized antibody. In embodiments, the heavy chain variable (VH) domain forms part of a humanized antibody. In embodiments, the light chain variable (VL) domain forms part of a chimeric antibody. In embodiments, the heavy chain variable (VH) domain forms part of a chimeric antibody. In embodiments, the light chain variable (VL) domain forms part of an antibody fragment. In embodiments, the heavy chain variable (VH) domain forms part of an antibody fragment. In embodiments, the light chain variable (VL) domain forms part of an antibody variant. In embodiments, the heavy chain variable (VH) domain forms part of an antibody variant. In embodiments, the light chain variable (VL) domain forms part of a Fab. In embodiments, the heavy chain variable (VH) domain forms part of a Fab. In embodiments, the light chain variable (VL) domain forms part of a scFv. In embodiments, the heavy chain variable (VH) domain forms part of a scFv.

In embodiments, the antibody is a humanized antibody. In embodiments, the antibody is a chimeric antibody. In embodiments, the antibody is a full length antibody. In embodiments, the antibody is a F(ab)2 fragment. In embodiments, the antibody is a Fab fragment. In embodiments, the antibody is a single chain antibody (scFv). In embodiments, the light chain variable domain and the heavy chain variable domain form part of a scFv. In embodiments, the full length antibody, F(ab)2 fragment, scFv, or Fab fragment includes the sequence of SEQ ID NO: 1. In embodiments, the full length antibody, F(ab)2 fragment, scFv, or Fab′ fragment includes the sequence of SEQ ID NO:2. In embodiments, the full length antibody, F(ab)2 fragment, scFv, or Fab′ fragment includes the sequence of SEQ ID NO:3. In embodiments, the full length antibody, F(ab)2 fragment, scFv, or Fab′ fragment includes the sequence of SEQ ID NO:4. In embodiments, the full length antibody, F(ab)2 fragment, scFv, or Fab fragment includes the sequence of SEQ ID NO:5. In embodiments, the full length antibody. F(ab)2 fragment, scFv, or Fab fragment includes the sequence of SEQ ID NO:6. In embodiments, the full length antibody, F(ab)2 fragment, scFv, or Fab′ fragment includes the sequence of SEQ ID NO:7. In embodiments, the full length antibody, F(ab)2 fragment, scFv, or Fab′ fragment includes the sequence of SEQ ID NO:8. In embodiments, the full length antibody, F(ab)2 fragment, scFv, or Fab fragment includes the sequence of SEQ ID NO:7 and the sequence of SEQ ID NO:8. In embodiments, the full length antibody, F(ab)2 fragment, scFv, or Fab′ fragment includes the sequence of SEQ ID NO:9. In embodiments, the full length antibody, F(ab)2 fragment, scFv, or Fab′ fragment includes the sequence of SEQ ID NO:10. In embodiments, the full length antibody, F(ab)2 fragment, scFv, or Fab fragment includes the sequence of SEQ ID NO:11. In embodiments, the full length antibody, F(ab)2 fragment, scFv, or Fab′ fragment includes the sequence of SEQ ID NO: 12. In embodiments, the full length antibody, F(ab)2 fragment, scFv, or Fab fragment includes the sequence of SEQ ID NO: 13. In embodiments, the full length antibody, F(ab)2 fragment, scFv, or Fab fragment includes the sequence of SEQ ID NO: 14. In embodiments, the full length antibody, F(ab)2 fragment, scFv, or Fab′ fragment includes the sequence of SEQ ID NO:15. In embodiments, the full length antibody, F(ab)2 fragment, scFv, or Fab′ fragment includes the sequence of SEQ ID NO: 16. In embodiments, the full length antibody, F(ab)2 fragment, scFv, or Fab fragment includes the sequence of SEQ ID NO: 15 and the sequence of SEQ ID NO:16. In embodiments, the full length antibody, F(ab)2 fragment, scFv, or Fab fragment includes the sequence of SEQ ID NO:17. In embodiments, the full length antibody, F(ab)2 fragment, scFv, or Fab fragment includes the sequence of SEQ ID NO: 18. In embodiments, the full length antibody, F(ab)2 fragment, scFv, or Fab fragment includes the sequence of SEQ ID NO:19. In embodiments, the full length antibody, F(ab)2 fragment, scFv, or Fab′ fragment includes the sequence of SEQ ID NO:20. In embodiments, the full length antibody, F(ab)2 fragment, scFv, or Fab fragment includes the sequence of SEQ ID NO:21. In embodiments, the full length antibody, F(ab)2 fragment, scFv, or Fab fragment includes the sequence of SEQ ID NO:22. In embodiments, the full length antibody, F(ab)2 fragment, scFv, or Fab′ fragment includes the sequence of SEQ ID NO:23. In embodiments, the full length antibody, F(ab)2 fragment, scFv, or Fab′ fragment includes the sequence of SEQ ID NO:24. In embodiments, the full length antibody, F(ab)2 fragment, scFv, or Fab′ fragment includes the sequence of SEQ ID NO:23 and the sequence of SEQ ID NO:24. In embodiments, the full length antibody, F(ab)2 fragment, scFv, or Fab fragment includes the sequence of SEQ ID NO:25. In embodiments, the full length antibody, F(ab)2 fragment, scFv, or Fab fragment includes the sequence of SEQ ID NO:26. In embodiments, the full length antibody, F(ab)2 fragment, scFv, or Fab fragment includes the sequence of SEQ ID NO:27. In embodiments, the full length antibody, F(ab)2 fragment, scFv, or Fab′ fragment includes the sequence of SEQ ID NO:28. In embodiments, the full length antibody, F(ab)2 fragment, scFv, or Fab fragment includes the sequence of SEQ ID NO:29. In embodiments, the full length antibody, F(ab)2 fragment, scFv, or Fab′ fragment includes the sequence of SEQ ID NO:30. In embodiments, the full length antibody, F(ab)2 fragment, scFv, or Fab fragment includes the sequence of SEQ ID NO:31. In embodiments, the full length antibody, F(ab)2 fragment, scFv, or Fab′ fragment includes the sequence of SEQ ID NO:32. In embodiments, the full length antibody, F(ab)2 fragment, scFv, or Fab fragment includes the sequence of SEQ ID NO:31 and the sequence of SEQ ID NO:32. In embodiments, the full length antibody, F(ab)2 fragment, scFv, or Fab fragment includes the sequence of SEQ ID NO:33. In embodiments, the full length antibody, F(ab)2 fragment, scFv, or Fab′ fragment includes the sequence of SEQ ID NO:34. In embodiments, the full length antibody, F(ab)2 fragment, scFv, or Fab fragment includes the sequence of SEQ ID NO:35. In embodiments, the full length antibody, F(ab)2 fragment, scFv, or Fab′ fragment includes the sequence of SEQ ID NO:36. In embodiments, the full length antibody, F(ab)2 fragment, scFv, or Fab fragment includes the sequence of SEQ ID NO:37. In embodiments, the full length antibody, F(ab)2 fragment, scFv, or Fab′ fragment includes the sequence of SEQ ID NO:38. In embodiments, the full length antibody, F(ab)2 fragment, scFv, or Fab fragment includes the sequence of SEQ ID NO:39. In embodiments, the full length antibody, F(ab)2 fragment, scFv, or Fab′ fragment includes the sequence of SEQ ID NO:40. In embodiments, the full length antibody, F(ab)2 fragment, scFv, or Fab′ fragment includes the sequence of SEQ ID NO:39 and the sequence of SEQ ID NO:40. In embodiments, the full length antibody, F(ab)2 fragment, scFv, or Fab fragment includes the sequence of SEQ ID NO:41. In embodiments, the full length antibody, F(ab)2 fragment, scFv, or Fab′ fragment includes the sequence of SEQ ID NO:42. In embodiments, the full length antibody, F(ab)2 fragment, scFv, or Fab fragment includes the sequence of SEQ ID NO:43. In embodiments, the full length antibody, F(ab)2 fragment, scFv, or Fab fragment includes the sequence of SEQ ID NO:44. In embodiments, the full length antibody, F(ab)2 fragment, scFv, or Fab′ fragment includes the sequence of SEQ ID NO:45. In embodiments, the full length antibody, F(ab)2 fragment, scFv, or Fab fragment includes the sequence of SEQ ID NO:46. In embodiments, the full length antibody, F(ab)2 fragment, scFv, or Fab fragment includes the sequence of SEQ ID NO:47. In embodiments, the full length antibody, F(ab)2 fragment, scFv, or Fab fragment includes the sequence of SEQ ID NO:48. In embodiments, the full length antibody, F(ab)2 fragment, scFv, or Fab fragment includes the sequence of SEQ ID NO:47 and the sequence of SEQ ID NO:48. In embodiments, the full length antibody, F(ab)2 fragment, scFv, or Fab fragment includes the sequence of SEQ ID NO:49. In embodiments, the full length antibody, F(ab)2 fragment, scFv, or Fab′ fragment includes the sequence of SEQ ID NO:50. In embodiments, the full length antibody, F(ab)2 fragment, scFv, or Fab fragment includes the sequence of SEQ ID NO:51. In embodiments, the full length antibody, F(ab)2 fragment, scFv, or Fab′ fragment includes the sequence of SEQ ID NO:52. In embodiments, the full length antibody, F(ab)2 fragment, scFv, or Fab fragment includes the sequence of SEQ ID NO:53. In embodiments, the full length antibody, F(ab)2 fragment, scFv, or Fab fragment includes the sequence of SEQ ID NO:54. In embodiments, the full length antibody, F(ab)2 fragment, scFv, or Fab′ fragment includes the sequence of SEQ ID NO:55. In embodiments, the full length antibody, F(ab)2 fragment, scFv, or Fab′ fragment includes the sequence of SEQ ID NO:56. In embodiments, the full length antibody, F(ab)2 fragment, scFv, or Fab′ fragment includes the sequence of SEQ ID NO:55 and the sequence of SEQ ID NO:56. In embodiments, the full length antibody, F(ab)2 fragment, scFv, or Fab′ fragment includes the sequence of SEQ ID NO:57. In embodiments, the full length antibody, F(ab)2 fragment, scFv, or Fab fragment includes the sequence of SEQ ID NO:58. In embodiments, the full length antibody, F(ab)2 fragment, scFv, or Fab′ fragment includes the sequence of SEQ ID NO:59. In embodiments, the full length antibody, F(ab)2 fragment, scFv, or Fab fragment includes the sequence of SEQ ID NO:60. In embodiments, the full length antibody, F(ab)2 fragment, scFv, or Fab fragment includes the sequence of SEQ ID NO:61. In embodiments, the full length antibody, F(ab)2 fragment, scFv, or Fab fragment includes the sequence of SEQ ID NO:62. In embodiments, the full length antibody, F(ab)2 fragment, scFv, or Fab fragment includes the sequence of SEQ ID NO:63. In embodiments, the full length antibody, F(ab)2 fragment, scFv, or Fab fragment includes the sequence of SEQ ID NO:64. In embodiments, the full length antibody, F(ab)2 fragment, scFv, or Fab fragment includes the sequence of SEQ ID NO:63 and the sequence of SEQ ID NO:64. In embodiments, the full length antibody, F(ab)2 fragment, scFv, or Fab fragment includes the sequence of SEQ ID NO:65. In embodiments, the full length antibody, F(ab)2 fragment, scFv, or Fab fragment includes the sequence of SEQ ID NO:66. In embodiments, the full length antibody, F(ab)2 fragment, scFv, or Fab fragment includes the sequence of SEQ ID NO:67. In embodiments, the full length antibody, F(ab)2 fragment, scFv, or Fab′ fragment includes the sequence of SEQ ID NO:68. In embodiments, the full length antibody, F(ab)2 fragment, scFv, or Fab fragment includes the sequence of SEQ ID NO:69. In embodiments, the full length antibody, F(ab)2 fragment, scFv, or Fab′ fragment includes the sequence of SEQ ID NO: 70. In embodiments, the full length antibody, F(ab)2 fragment, scFv, or Fab′ fragment includes the sequence of SEQ ID NO: 71. In embodiments, the full length antibody, F(ab)2 fragment, scFv, or Fab fragment includes the sequence of SEQ ID NO:72. In embodiments, the full length antibody, F(ab)2 fragment, scFv, or Fab fragment includes the sequence of SEQ ID NO:71 and the sequence of SEQ ID NO:72. In embodiments, at least a portion of the full length antibody, F(ab)2 fragment, scFv, or Fab fragment is encoded by the sequence of SEQ ID NO:73. In embodiments, at least a portion of the full length antibody, F(ab)2 fragment, scFv, or Fab fragment is encoded by the sequence of SEQ ID NO:74. In embodiments, at least a portion of the full length antibody, F(ab)2 fragment, scFv, or Fab′ fragment is encoded by the sequence of SEQ ID NO: 75. In embodiments, at least a portion of the full length antibody, F(ab)2 fragment, scFv, or Fab fragment is encoded by the sequence of SEQ ID NO:76. In embodiments, at least a portion of the full length antibody, F(ab)2 fragment, scFv, or Fab′ fragment is encoded by the sequence of SEQ ID NO:77. In embodiments, at least a portion of the full length antibody, F(ab)2 fragment, scFv, or Fab fragment is encoded by the sequence of SEQ ID NO:78. In embodiments, at least a portion of the full length antibody. F(ab)2 fragment, scFv, or Fab′ fragment is encoded by the sequence of SEQ ID NO:79. In embodiments, at least a portion of the full length antibody, F(ab)2 fragment, scFv, or Fab fragment is encoded by the sequence of SEQ ID NO:80. In embodiments, a first portion of the full length antibody, F(ab)2 fragment, scFv, or Fab′ fragment is encoded by the sequence of SEQ ID NO:79 and a second portion of the full length antibody, F(ab)2 fragment, scFv, or Fab′ fragment is encoded by the sequence of SEQ ID NO:80. In embodiments, the full length antibody, F(ab)2 fragment, scFv, or Fab′ fragment includes the sequence of SEQ ID NO:81. In embodiments, the full length antibody, F(ab)2 fragment, scFv, or Fab fragment includes the sequence of SEQ ID NO:82. In embodiments, the full length antibody, F(ab)2 fragment, scFv, or Fab fragment includes the sequence of SEQ ID NO:83. In embodiments, the full length antibody, F(ab)2 fragment, scFv, or Fab′ fragment includes the sequence of SEQ ID NO:84. In embodiments, the full length antibody, F(ab)2 fragment, scFv, or Fab fragment includes the sequence of SEQ ID NO:85. In embodiments, the full length antibody, F(ab)2 fragment, scFv, or Fab′ fragment includes the sequence of SEQ ID NO:86. In embodiments, the full length antibody, F(ab)2 fragment, scFv, or Fab′ fragment includes the sequence of SEQ ID NO:87. In embodiments, the full length antibody, F(ab)2 fragment, scFv, or Fab fragment includes the sequence of SEQ ID NO:88. In embodiments, the full length antibody, F(ab)2 fragment, scFv, or Fab fragment includes the sequence of SEQ ID NO:87 and the sequence of SEQ ID NO:88. In embodiments, the full length antibody, F(ab)2 fragment, scFv, or Fab′ fragment includes the sequence of SEQ ID NO:89. In embodiments, the full length antibody, F(ab)2 fragment, scFv, or Fab fragment includes the sequence of SEQ ID NO:90. In embodiments, the full length antibody, F(ab)2 fragment, scFv, or Fab fragment includes the sequence of SEQ ID NO:91. In embodiments, the full length antibody, F(ab)2 fragment, scFv, or Fab fragment includes the sequence of SEQ ID NO:92. In embodiments, the full length antibody, F(ab)2 fragment, scFv, or Fab fragment includes the sequence of SEQ ID NO:93. In embodiments, the full length antibody, F(ab)2 fragment, scFv, or Fab fragment includes the sequence of SEQ ID NO:94. In embodiments, the full length antibody, F(ab)2 fragment, scFv, or Fab′ fragment includes the sequence of SEQ ID NO:95. In embodiments, the full length antibody, F(ab)2 fragment, scFv, or Fab fragment includes the sequence of SEQ ID NO:96. In embodiments, the full length antibody, F(ab)2 fragment, scFv, or Fab fragment includes the sequence of SEQ ID NO:95 and the sequence of SEQ ID NO:96. In embodiments, the full length antibody, F(ab)2 fragment, scFv, or Fab′ fragment includes the sequence of SEQ ID NO:97. In embodiments, the full length antibody, F(ab)2 fragment, scFv, or Fab′ fragment includes the sequence of SEQ ID NO:98. In embodiments, the full length antibody, F(ab)2 fragment, scFv, or Fab fragment includes the sequence of SEQ ID NO:99. In embodiments, the full length antibody, F(ab)2 fragment, scFv, or Fab′ fragment includes the sequence of SEQ ID NO: 100. In embodiments, the full length antibody, F(ab)2 fragment, scFv, or Fab fragment includes the sequence of SEQ ID NO:101. In embodiments, the full length antibody, F(ab)2 fragment, scFv, or Fab fragment includes the sequence of SEQ ID NO: 102. In embodiments, the full length antibody, F(ab)2 fragment, scFv, or Fab fragment includes the sequence of SEQ ID NO: 103. In embodiments, the full length antibody, F(ab)2 fragment, scFv, or Fab′ fragment includes the sequence of SEQ ID NO: 104. In embodiments, the full length antibody, F(ab)2 fragment, scFv, or Fab′ fragment includes the sequence of SEQ ID NO: 103 and the sequence of SEQ ID NO: 104. In embodiments, the full length antibody, F(ab)2 fragment, scFv, or Fab′ fragment includes the sequence of SEQ ID NO: 105. In embodiments, the full length antibody, F(ab)2 fragment, scFv, or Fab fragment includes the sequence of SEQ ID NO: 106. In embodiments, the full length antibody, F(ab)2 fragment, scFv, or Fab fragment includes the sequence of SEQ ID NO:107. In embodiments, the full length antibody, F(ab)2 fragment, scFv, or Fab fragment includes the sequence of SEQ ID NO: 108. In embodiments, the full length antibody, F(ab)2 fragment, scFv, or Fab fragment includes the sequence of SEQ ID NO: 109. In embodiments, the full length antibody, F(ab)2 fragment, scFv, or Fab fragment includes the sequence of SEQ ID NO: 110. In embodiments, the full length antibody, F(ab)2 fragment, scFv, or Fab′ fragment includes the sequence of SEQ ID NO:111. In embodiments, the full length antibody, F(ab)2 fragment, scFv, or Fab fragment includes the sequence of SEQ ID NO:112. In embodiments, the full length antibody, F(ab)2 fragment, scFv, or Fab fragment includes the sequence of SEQ ID NO: 111 and the sequence of SEQ ID NO:112. In embodiments, the full length antibody, F(ab)2 fragment, scFv, or Fab′ fragment includes the sequence of SEQ ID NO:113. In embodiments, the full length antibody, F(ab)2 fragment, scFv, or Fab′ fragment includes the sequence of SEQ ID NO: 114. In embodiments, the full length antibody, F(ab)2 fragment, scFv, or Fab fragment includes the sequence of SEQ ID NO: 115. In embodiments, the full length antibody, F(ab)2 fragment, scFv, or Fab′ fragment includes the sequence of SEQ ID NO: 116. In embodiments, the full length antibody, F(ab)2 fragment, scFv, or Fab fragment includes the sequence of SEQ ID NO:117. In embodiments, the full length antibody, F(ab)2 fragment, scFv, or Fab fragment includes the sequence of SEQ ID NO: 118. In embodiments, the full length antibody, F(ab)2 fragment, scFv, or Fab fragment includes the sequence of SEQ ID NO: 119. In embodiments, the full length antibody, F(ab)2 fragment, scFv, or Fab fragment includes the sequence of SEQ ID NO: 120. In embodiments, the full length antibody, F(ab)2 fragment, scFv, or Fab′ fragment includes the sequence of SEQ ID NO: 119 and the sequence of SEQ ID NO:120. In embodiments, the full length antibody, F(ab)2 fragment, scFv, or Fab′ fragment includes the sequence of SEQ ID NO: 121. In embodiments, the full length antibody, F(ab)2 fragment, scFv, or Fab′ fragment includes the sequence of SEQ ID NO: 122. In embodiments, the full length antibody, F(ab)2 fragment, scFv, or Fab′ fragment includes the sequence of SEQ ID NO:123. In embodiments, the full length antibody, F(ab)2 fragment, scFv, or Fab′ fragment includes the sequence of SEQ ID NO: 124. In embodiments, the full length antibody, F(ab)2 fragment, scFv, or Fab fragment includes the sequence of SEQ ID NO: 125. In embodiments, the full length antibody, F(ab)2 fragment, scFv, or Fab′ fragment includes the sequence of SEQ ID NO: 126. In embodiments, the full length antibody, F(ab)2 fragment, scFv, or Fab′ fragment includes the sequence of SEQ ID NO:127. In embodiments, the full length antibody, F(ab)2 fragment, scFv, or Fab fragment includes the sequence of SEQ ID NO: 128. In embodiments, the full length antibody, F(ab)2 fragment, scFv, or Fab fragment includes the sequence of SEQ ID NO: 127 and the sequence of SEQ ID NO: 128. In embodiments, the full length antibody, F(ab)2 fragment, scFv, or Fab′ fragment includes the sequence of SEQ ID NO: 129. In embodiments, the full length antibody, F(ab)2 fragment, scFv, or Fab′ fragment includes the sequence of SEQ ID NO:130. In embodiments, the full length antibody, F(ab)2 fragment, scFv, or Fab′ fragment includes the sequence of SEQ ID NO:131. In embodiments, the full length antibody, F(ab)2 fragment, scFv, or Fab′ fragment includes the sequence of SEQ ID NO: 132. In embodiments, the full length antibody, F(ab)2 fragment, scFv, or Fab′ fragment includes the sequence of SEQ ID NO:133. In embodiments, the full length antibody, F(ab)2 fragment, scFv, or Fab fragment includes the sequence of SEQ ID NO:134. In embodiments, the full length antibody, F(ab)2 fragment, scFv, or Fab′ fragment includes the sequence of SEQ ID NO: 135. In embodiments, the full length antibody, F(ab)2 fragment, scFv, or Fab′ fragment includes the sequence of SEQ ID NO: 136. In embodiments, the full length antibody, F(ab)2 fragment, scFv, or Fab′ fragment includes the sequence of SEQ ID NO: 135 and the sequence of SEQ ID NO: 136. In embodiments, the full length antibody, F(ab)2 fragment, scFv, or Fab fragment includes the sequence of SEQ ID NO: 137. In embodiments, the full length antibody, F(ab)2 fragment, scFv, or Fab fragment includes the sequence of SEQ ID NO:138. In embodiments, the full length antibody, F(ab)2 fragment, scFv, or Fab fragment includes the sequence of SEQ ID NO:139. In embodiments, the full length antibody, F(ab)2 fragment, scFv, or Fab fragment includes the sequence of SEQ ID NO: 140. In embodiments, the full length antibody, F(ab)2 fragment, scFv, or Fab′ fragment includes the sequence of SEQ ID NO: 141. In embodiments, the full length antibody, F(ab)2 fragment, scFv, or Fab fragment includes the sequence of SEQ ID NO: 142. In embodiments, the full length antibody, F(ab)2 fragment, scFv, or Fab fragment includes the sequence of SEQ ID NO:143. In embodiments, the full length antibody, F(ab)2 fragment, scFv, or Fab fragment includes the sequence of SEQ ID NO:144. In embodiments, the full length antibody, F(ab)2 fragment, scFv, or Fab fragment includes the sequence of SEQ ID NO: 143 and the sequence of SEQ ID NO: 144. In embodiments, the full length antibody, F(ab)2 fragment, scFv, or Fab′ fragment includes the sequence of SEQ ID NO:145. In embodiments, the full length antibody, F(ab)2 fragment, scFv, or Fab fragment includes the sequence of SEQ ID NO: 146. In embodiments, the full length antibody, F(ab)2 fragment, scFv, or Fab fragment includes the sequence of SEQ ID NO: 147. In embodiments, the full length antibody, F(ab)2 fragment, scFv, or Fab fragment includes the sequence of SEQ ID NO: 148. In embodiments, the full length antibody, F(ab)2 fragment, scFv, or Fab′ fragment includes the sequence of SEQ ID NO:149. In embodiments, the full length antibody, F(ab)2 fragment, scFv, or Fab fragment includes the sequence of SEQ ID NO:150. In embodiments, the full length antibody, F(ab)2 fragment, scFv, or Fab′ fragment includes the sequence of SEQ ID NO: 151. In embodiments, the full length antibody, F(ab)2 fragment, scFv, or Fab fragment includes the sequence of SEQ ID NO: 152. In embodiments, the full length antibody, F(ab)2 fragment, scFv, or Fab fragment includes the sequence of SEQ ID NO: 151 and the sequence of SEQ ID NO: 152. In embodiments, at least a portion of the full length antibody, F(ab)2 fragment, scFv, or Fab′ fragment is encoded by the sequence of SEQ ID NO: 153. In embodiments, at least a portion of the full length antibody, F(ab)2 fragment, scFv, or Fab fragment is encoded by the sequence of SEQ ID NO:154. In embodiments, at least a portion of the full length antibody, F(ab)2 fragment, scFv, or Fab′ fragment is encoded by the sequence of SEQ ID NO: 155. In embodiments, at least a portion of the full length antibody, F(ab)2 fragment, scFv, or Fab fragment is encoded by the sequence of SEQ ID NO: 156. In embodiments, at least a portion of the full length antibody, F(ab)2 fragment, scFv, or Fab fragment is encoded by the sequence of SEQ ID NO: 157. In embodiments, at least a portion of the full length antibody, F(ab)2 fragment, scFv, or Fab′ fragment is encoded by the sequence of SEQ ID NO:158. In embodiments, at least a portion of the full length antibody, F(ab)2 fragment, scFv, or Fab fragment is encoded by the sequence of SEQ ID NO: 159. In embodiments, at least a portion of the full length antibody, F(ab)2 fragment, scFv, or Fab′ fragment is encoded by the sequence of SEQ ID NO: 160. In embodiments, a first portion of the full length antibody. F(ab)2 fragment, scFv, or Fab′ fragment is encoded by the sequence of SEQ ID NO: 159 and a second portion of the full length antibody, F(ab)2 fragment, scFv, or Fab fragment is encoded by the sequence of SEQ ID NO:160. In embodiments, the full length antibody, F(ab)2 fragment, scFv, or Fab fragment includes the sequence of SEQ ID NO: 161. In embodiments, the full length antibody, F(ab)2 fragment, scFv, or Fab fragment includes the sequence of SEQ ID NO: 162. In embodiments, the full length antibody, F(ab)2 fragment, scFv, or Fab′ fragment includes the sequence of SEQ ID NO: 163. In embodiments, the full length antibody, F(ab)2 fragment, scFv, or Fab fragment includes the sequence of SEQ ID NO:164. In embodiments, the full length antibody, F(ab)2 fragment, scFv, or Fab fragment includes the sequence of SEQ ID NO: 165. In embodiments, the full length antibody, F(ab)2 fragment, scFv, or Fab′ fragment includes the sequence of SEQ ID NO: 166. In embodiments, the full length antibody, F(ab)2 fragment, scFv, or Fab fragment includes the sequence of SEQ ID NO:167. In embodiments, the full length antibody, F(ab)2 fragment, scFv, or Fab fragment includes the sequence of SEQ ID NO:168. In embodiments, the full length antibody, F(ab)2 fragment, scFv, or Fab fragment includes the sequence of SEQ ID NO: 167 and the sequence of SEQ ID NO:168. In embodiments, the full length antibody, F(ab)2 fragment, scFv, or Fab fragment includes the sequence of SEQ ID NO: 169. In embodiments, the full length antibody, F(ab)2 fragment, scFv, or Fab fragment includes the sequence of SEQ ID NO:170. In embodiments, the full length antibody, F(ab)2 fragment, scFv, or Fab fragment includes the sequence of SEQ ID NO:171. In embodiments, the full length antibody, F(ab)2 fragment, scFv, or Fab′ fragment includes the sequence of SEQ ID NO:172. In embodiments, the full length antibody, F(ab)2 fragment, scFv, or Fab fragment includes the sequence of SEQ ID NO: 173. In embodiments, the full length antibody, F(ab)2 fragment, scFv, or Fab′ fragment includes the sequence of SEQ ID NO:174. In embodiments, the full length antibody, F(ab)2 fragment, scFv, or Fab fragment includes the sequence of SEQ ID NO:175. In embodiments, the full length antibody, F(ab)2 fragment, scFv, or Fab fragment includes the sequence of SEQ ID NO: 176. In embodiments, the full length antibody, F(ab)2 fragment, scFv, or Fab′ fragment includes the sequence of SEQ ID NO: 175 and the sequence of SEQ ID NO:176. In embodiments, the full length antibody, F(ab)2 fragment, scFv, or Fab fragment includes the sequence of SEQ ID NO:177. In embodiments, the full length antibody, F(ab)2 fragment, scFv, or Fab fragment includes the sequence of SEQ ID NO: 178. In embodiments, the full length antibody, F(ab)2 fragment, scFv, or Fab′ fragment includes the sequence of SEQ ID NO: 179. In embodiments, the full length antibody, F(ab)2 fragment, scFv, or Fab fragment includes the sequence of SEQ ID NO:180. In embodiments, the full length antibody, F(ab)2 fragment, scFv, or Fab′ fragment includes the sequence of SEQ ID NO: 181. In embodiments, the full length antibody, F(ab)2 fragment, scFv, or Fab′ fragment includes the sequence of SEQ ID NO: 182. In embodiments, the full length antibody, F(ab)2 fragment, scFv, or Fab fragment includes the sequence of SEQ ID NO: 183. In embodiments, the full length antibody, F(ab)2 fragment, scFv, or Fab′ fragment includes the sequence of SEQ ID NO:184. In embodiments, the full length antibody, F(ab)2 fragment, scFv, or Fab fragment includes the sequence of SEQ ID NO: 183 and the sequence of SEQ ID NO: 184. In embodiments, the full length antibody, F(ab)2 fragment, scFv, or Fab′ fragment includes the sequence of SEQ ID NO: 185. In embodiments, the full length antibody, F(ab)2 fragment, scFv, or Fab′ fragment includes the sequence of SEQ ID NO:186. In embodiments, the full length antibody, F(ab)2 fragment, scFv, or Fab fragment includes the sequence of SEQ ID NO: 187. In embodiments, the full length antibody, F(ab)2 fragment, scFv, or Fab fragment includes the sequence of SEQ ID NO: 188. In embodiments, the full length antibody, F(ab)2 fragment, scFv, or Fab′ fragment includes the sequence of SEQ ID NO: 189. In embodiments, the full length antibody, F(ab)2 fragment, scFv, or Fab′ fragment includes the sequence of SEQ ID NO:190. In embodiments, the full length antibody, F(ab)2 fragment, scFv, or Fab′ fragment includes the sequence of SEQ ID NO:191. In embodiments, the full length antibody, F(ab)2 fragment, scFv, or Fab fragment includes the sequence of SEQ ID NO:32. In embodiments, the full length antibody, F(ab)2 fragment, scFv, or Fab fragment includes the sequence of SEQ ID NO: 191 and the sequence of SEQ ID NO: 192. In embodiments, the full length antibody, F(ab)2 fragment, scFv, or Fab fragment includes the sequence of SEQ ID NO: 193. In embodiments, the full length antibody, F(ab)2 fragment, scFv, or Fab fragment includes the sequence of SEQ ID NO: 194. In embodiments, the full length antibody, F(ab)2 fragment, scFv, or Fab′ fragment includes the sequence of SEQ ID NO: 195. In embodiments, the full length antibody, F(ab)2 fragment, scFv, or Fab′ fragment includes the sequence of SEQ ID NO:196. In embodiments, the full length antibody, F(ab)2 fragment, scFv, or Fab′ fragment includes the sequence of SEQ ID NO: 197. In embodiments, the full length antibody, F(ab)2 fragment, scFv, or Fab′ fragment includes the sequence of SEQ ID NO: 198. In embodiments, the full length antibody, F(ab)2 fragment, scFv, or Fab′ fragment includes the sequence of SEQ ID NO: 199. In embodiments, the full length antibody, F(ab)2 fragment, scFv, or Fab fragment includes the sequence of SEQ ID NO:200. In embodiments, the full length antibody, F(ab)2 fragment, scFv, or Fab fragment includes the sequence of SEQ ID NO: 199 and the sequence of SEQ ID NO:200. In embodiments, the full length antibody, F(ab)2 fragment, scFv, or Fab fragment includes the sequence of SEQ ID NO:201. In embodiments, the full length antibody, F(ab)2 fragment, scFv, or Fab fragment includes the sequence of SEQ ID NO:202. In embodiments, the full length antibody, F(ab)2 fragment, scFv, or Fab fragment includes the sequence of SEQ ID NO:203. In embodiments, the full length antibody, F(ab)2 fragment, scFv, or Fab fragment includes the sequence of SEQ ID NO:204. In embodiments, the full length antibody, F(ab)2 fragment, scFv, or Fab fragment includes the sequence of SEQ ID NO:205. In embodiments, the full length antibody, F(ab)2 fragment, scFv, or Fab fragment includes the sequence of SEQ ID NO:206. In embodiments, the full length antibody, F(ab)2 fragment, scFv, or Fab fragment includes the sequence of SEQ ID NO:207. In embodiments, the full length antibody, F(ab)2 fragment, scFv, or Fab fragment includes the sequence of SEQ ID NO:208. In embodiments, the full length antibody, F(ab)2 fragment, scFv, or Fab′ fragment includes the sequence of SEQ ID NO:207 and the sequence of SEQ ID NO:208. In embodiments, the full length antibody, F(ab)2 fragment, scFv, or Fab fragment includes the sequence of SEQ ID NO:209. In embodiments, the full length antibody, F(ab)2 fragment, scFv, or Fab′ fragment includes the sequence of SEQ ID NO:210. In embodiments, the full length antibody, F(ab)2 fragment, scFv, or Fab′ fragment includes the sequence of SEQ ID NO:211. In embodiments, the full length antibody, F(ab)2 fragment, scFv, or Fab′ fragment includes the sequence of SEQ ID NO:212. In embodiments, the full length antibody, F(ab)2 fragment, scFv, or Fab fragment includes the sequence of SEQ ID NO:213. In embodiments, the full length antibody, F(ab)2 fragment, scFv, or Fab′ fragment includes the sequence of SEQ ID NO:214. In embodiments, the full length antibody, F(ab)2 fragment, scFv, or Fab fragment includes the sequence of SEQ ID NO:215. In embodiments, the full length antibody, F(ab)2 fragment, scFv, or Fab′ fragment includes the sequence of SEQ ID NO:216. In embodiments, the full length antibody, F(ab)2 fragment, scFv, or Fab fragment includes the sequence of SEQ ID NO:215 and the sequence of SEQ ID NO:216. In embodiments, the full length antibody, F(ab)2 fragment, scFv, or Fab′ fragment includes the sequence of SEQ ID NO:217. In embodiments, the full length antibody, F(ab)2 fragment, scFv, or Fab fragment includes the sequence of SEQ ID NO:218. In embodiments, the full length antibody, F(ab)2 fragment, scFv, or Fab′ fragment includes the sequence of SEQ ID NO:219. In embodiments, the full length antibody, F(ab)2 fragment, scFv, or Fab fragment includes the sequence of SEQ ID NO:220. In embodiments, the full length antibody, F(ab)2 fragment, scFv, or Fab fragment includes the sequence of SEQ ID NO:221. In embodiments, the full length antibody, F(ab)2 fragment, scFv, or Fab′ fragment includes the sequence of SEQ ID NO:222. In embodiments, the full length antibody, F(ab)2 fragment, scFv, or Fab′ fragment includes the sequence of SEQ ID NO:223. In embodiments, the full length antibody, F(ab)2 fragment, scFv, or Fab′ fragment includes the sequence of SEQ ID NO:224. In embodiments, the full length antibody, F(ab)2 fragment, scFv, or Fab′ fragment includes the sequence of SEQ ID NO:223 and the sequence of SEQ ID NO:224. In embodiments, the full length antibody, F(ab)2 fragment, scFv, or Fab′ fragment includes the sequence of SEQ ID NO:225. In embodiments, the full length antibody, F(ab)2 fragment, scFv, or Fab′ fragment includes the sequence of SEQ ID NO:226. In embodiments, the full length antibody, F(ab)2 fragment, scFv, or Fab′ fragment includes the sequence of SEQ ID NO:227. In embodiments, the full length antibody, F(ab)2 fragment, scFv, or Fab fragment includes the sequence of SEQ ID NO:228. In embodiments, the full length antibody, F(ab)2 fragment, scFv, or Fab fragment includes the sequence of SEQ ID NO:229. In embodiments, the full length antibody, F(ab)2 fragment, scFv, or Fab′ fragment includes the sequence of SEQ ID NO:230. In embodiments, the full length antibody, F(ab)2 fragment, scFv, or Fab fragment includes the sequence of SEQ ID NO:231. In embodiments, the full length antibody, F(ab)2 fragment, scFv, or Fab′ fragment includes the sequence of SEQ ID NO:232. In embodiments, the full length antibody, F(ab)2 fragment, scFv, or Fab fragment includes the sequence of SEQ ID NO:231 and the sequence of SEQ ID NO:232. In embodiments, at least a portion of the full length antibody, F(ab)2 fragment, scFv, or Fab′ fragment is encoded by the sequence of SEQ ID NO:233. In embodiments, at least a portion of the full length antibody, F(ab)2 fragment, scFv, or Fab′ fragment is encoded by the sequence of SEQ ID NO:234. In embodiments, at least a portion of the full length antibody, F(ab)2 fragment, scFv, or Fab′ fragment is encoded by the sequence of SEQ ID NO: 235. In embodiments, at least a portion of the full length antibody, F(ab)2 fragment, scFv, or Fab′ fragment is encoded by the sequence of SEQ ID NO:236. In embodiments, at least a portion of the full length antibody, F(ab)2 fragment, scFv, or Fab′ fragment is encoded by the sequence of SEQ ID NO: 237. In embodiments, at least a portion of the full length antibody, F(ab)2 fragment, scFv, or Fab fragment is encoded by the sequence of SEQ ID NO:238. In embodiments, at least a portion of the full length antibody, F(ab)2 fragment, scFv, or Fab′ fragment is encoded by the sequence of SEQ ID NO:239. In embodiments, at least a portion of the full length antibody, F(ab)2 fragment, scFv, or Fab fragment is encoded by the sequence of SEQ ID NO:240. In embodiments, a first portion of the full length antibody. F(ab)2 fragment, scFv, or Fab′ fragment is encoded by the sequence of SEQ ID NO:239 and a second portion of the full length antibody, F(ab)2 fragment, scFv, or Fab fragment is encoded by the sequence of SEQ ID NO:240. In embodiments, the full length antibody, F(ab)2 fragment, scFv, or Fab fragment includes the sequence of SEQ ID NO:241. In embodiments, the full length antibody, F(ab)2 fragment, scFv, or Fab fragment includes the sequence of SEQ ID NO:242. In embodiments, the full length antibody, F(ab)2 fragment, scFv, or Fab′ fragment includes the sequence of SEQ ID NO:243. In embodiments, the full length antibody, F(ab)2 fragment, scFv, or Fab fragment includes the sequence of SEQ ID NO:244. In embodiments, the full length antibody, F(ab)2 fragment, scFv, or Fab fragment includes the sequence of SEQ ID NO:245. In embodiments, the full length antibody, F(ab)2 fragment, scFv, or Fab′ fragment includes the sequence of SEQ ID NO:246. In embodiments, the full length antibody, F(ab)2 fragment, scFv, or Fab fragment includes the sequence of SEQ ID NO:247. In embodiments, the full length antibody, F(ab)2 fragment, scFv, or Fab′ fragment includes the sequence of SEQ ID NO:248. In embodiments, the full length antibody, F(ab)2 fragment, scFv, or Fab fragment includes the sequence of SEQ ID NO:247 and the sequence of SEQ ID NO:248. In embodiments, the full length antibody, F(ab)2 fragment, scFv, or Fab′ fragment includes the sequence of SEQ ID NO:249. In embodiments, the full length antibody, F(ab)2 fragment, scFv, or Fab fragment includes the sequence of SEQ ID NO:250. In embodiments, the full length antibody, F(ab)2 fragment, scFv, or Fab fragment includes the sequence of SEQ ID NO:251. In embodiments, the full length antibody, F(ab)2 fragment, scFv, or Fab′ fragment includes the sequence of SEQ ID NO:252. In embodiments, the full length antibody, F(ab)2 fragment, scFv, or Fab fragment includes the sequence of SEQ ID NO:253. In embodiments, the full length antibody, F(ab)2 fragment, scFv, or Fab′ fragment includes the sequence of SEQ ID NO:254. In embodiments, the full length antibody, F(ab)2 fragment, scFv, or Fab′ fragment includes the sequence of SEQ ID NO:255. In embodiments, the full length antibody, F(ab)2 fragment, scFv, or Fab′ fragment includes the sequence of SEQ ID NO:256. In embodiments, the full length antibody, F(ab)2 fragment, scFv, or Fab fragment includes the sequence of SEQ ID NO:255 and the sequence of SEQ ID NO:256. In embodiments, the full length antibody, F(ab)2 fragment, scFv, or Fab fragment includes the sequence of SEQ ID NO:257. In embodiments, the full length antibody, F(ab)2 fragment, scFv, or Fab′ fragment includes the sequence of SEQ ID NO:258. In embodiments, the full length antibody, F(ab)2 fragment, scFv, or Fab′ fragment includes the sequence of SEQ ID NO:259. In embodiments, the full length antibody, F(ab)2 fragment, scFv, or Fab′ fragment includes the sequence of SEQ ID NO:260. In embodiments, the full length antibody, F(ab)2 fragment, scFv, or Fab fragment includes the sequence of SEQ ID NO:261. In embodiments, the full length antibody, F(ab)2 fragment, scFv, or Fab′ fragment includes the sequence of SEQ ID NO:262. In embodiments, the full length antibody, F(ab)2 fragment, scFv, or Fab′ fragment includes the sequence of SEQ ID NO: 263. In embodiments, the full length antibody, F(ab)2 fragment, scFv, or Fab fragment includes the sequence of SEQ ID NO:264. In embodiments, the full length antibody, F(ab)2 fragment, scFv, or Fab′ fragment includes the sequence of SEQ ID NO:263 and the sequence of SEQ ID NO:264. In embodiments, the full length antibody, F(ab)2 fragment, scFv, or Fab′ fragment includes the sequence of SEQ ID NO:265. In embodiments, the full length antibody, F(ab)2 fragment, scFv, or Fab′ fragment includes the sequence of SEQ ID NO:266. In embodiments, the full length antibody, F(ab)2 fragment, scFv, or Fab′ fragment includes the sequence of SEQ ID NO:267. In embodiments, the full length antibody, F(ab)2 fragment, scFv, or Fab fragment includes the sequence of SEQ ID NO:268. In embodiments, the full length antibody, F(ab)2 fragment, scFv, or Fab′ fragment includes the sequence of SEQ ID NO:269. In embodiments, the full length antibody, F(ab)2 fragment, scFv, or Fab fragment includes the sequence of SEQ ID NO:270. In embodiments, the full length antibody, F(ab)2 fragment, scFv, or Fab′ fragment includes the sequence of SEQ ID NO:271. In embodiments, the full length antibody, F(ab)2 fragment, scFv, or Fab′ fragment includes the sequence of SEQ ID NO:272. In embodiments, the full length antibody, F(ab)2 fragment, scFv, or Fab′ fragment includes the sequence of SEQ ID NO:271 and the sequence of SEQ ID NO:272. In embodiments, the full length antibody, F(ab)2 fragment, scFv, or Fab fragment includes the sequence of SEQ ID NO:273. In embodiments, the full length antibody, F(ab)2 fragment, scFv, or Fab fragment includes the sequence of SEQ ID NO:274. In embodiments, the full length antibody, F(ab)2 fragment, scFv, or Fab fragment includes the sequence of SEQ ID NO:275. In embodiments, the full length antibody, F(ab)2 fragment, scFv, or Fab fragment includes the sequence of SEQ ID NO:276. In embodiments, the full length antibody, F(ab)2 fragment, scFv, or Fab′ fragment includes the sequence of SEQ ID NO:277. In embodiments, the full length antibody, F(ab)2 fragment, scFv, or Fab fragment includes the sequence of SEQ ID NO:278. In embodiments, the full length antibody, F(ab)2 fragment, scFv, or Fab′ fragment includes the sequence of SEQ ID NO: 279. In embodiments, the full length antibody, F(ab)2 fragment, scFv, or Fab′ fragment includes the sequence of SEQ ID NO:280. In embodiments, the full length antibody, F(ab)2 fragment, scFv, or Fab fragment includes the sequence of SEQ ID NO:279 and the sequence of SEQ ID NO:280. In embodiments, the full length antibody, F(ab)2 fragment, scFv, or Fab fragment includes the sequence of SEQ ID NO:281. In embodiments, the full length antibody, F(ab)2 fragment, scFv, or Fab fragment includes the sequence of SEQ ID NO:282. In embodiments, the full length antibody, F(ab)2 fragment, scFv, or Fab′ fragment includes the sequence of SEQ ID NO:283. In embodiments, the full length antibody, F(ab)2 fragment, scFv, or Fab fragment includes the sequence of SEQ ID NO:284. In embodiments, the full length antibody, F(ab)2 fragment, scFv, or Fab′ fragment includes the sequence of SEQ ID NO: 285. In embodiments, the full length antibody, F(ab)2 fragment, scFv, or Fab′ fragment includes the sequence of SEQ ID NO:286. In embodiments, the full length antibody, F(ab)2 fragment, scFv, or Fab fragment includes the sequence of SEQ ID NO:287. In embodiments, the full length antibody, F(ab)2 fragment, scFv, or Fab fragment includes the sequence of SEQ ID NO:288. In embodiments, the full length antibody, F(ab)2 fragment, scFv, or Fab′ fragment includes the sequence of SEQ ID NO: 287 and the sequence of SEQ ID NO:288. In embodiments, the full length antibody, F(ab)2 fragment, scFv, or Fab′ fragment includes the sequence of SEQ ID NO:289. In embodiments, the full length antibody, F(ab)2 fragment, scFv, or Fab fragment includes the sequence of SEQ ID NO:290. In embodiments, the full length antibody, F(ab)2 fragment, scFv, or Fab fragment includes the sequence of SEQ ID NO:291. In embodiments, the full length antibody, F(ab)2 fragment, scFv, or Fab′ fragment includes the sequence of SEQ ID NO:292. In embodiments, the full length antibody, F(ab)2 fragment, scFv, or Fab fragment includes the sequence of SEQ ID NO:293. In embodiments, the full length antibody, F(ab)2 fragment, scFv, or Fab fragment includes the sequence of SEQ ID NO:294. In embodiments, the full length antibody, F(ab)2 fragment, scFv, or Fab fragment includes the sequence of SEQ ID NO:295. In embodiments, the full length antibody, F(ab)2 fragment, scFv, or Fab′ fragment includes the sequence of SEQ ID NO:296. In embodiments, the full length antibody, F(ab)2 fragment, scFv, or Fab fragment includes the sequence of SEQ ID NO:295 and the sequence of SEQ ID NO:296. In embodiments, the full length antibody, F(ab)2 fragment, scFv, or Fab fragment includes the sequence of SEQ ID NO:297. In embodiments, the full length antibody, F(ab)2 fragment, scFv, or Fab fragment includes the sequence of SEQ ID NO:298. In embodiments, the full length antibody, F(ab)2 fragment, scFv, or Fab fragment includes the sequence of SEQ ID NO:299. In embodiments, the full length antibody, F(ab)2 fragment, scFv, or Fab fragment includes the sequence of SEQ ID NO:300. In embodiments, the full length antibody, F(ab)2 fragment, scFv, or Fab′ fragment includes the sequence of SEQ ID NO: 301. In embodiments, the full length antibody, F(ab)2 fragment, scFv, or Fab′ fragment includes the sequence of SEQ ID NO:302. In embodiments, the full length antibody, F(ab)2 fragment, scFv, or Fab fragment includes the sequence of SEQ ID NO:303. In embodiments, the full length antibody, F(ab)2 fragment, scFv, or Fab fragment includes the sequence of SEQ ID NO:304. In embodiments, the full length antibody, F(ab)2 fragment, scFv, or Fab′ fragment includes the sequence of SEQ ID NO:303 and the sequence of SEQ ID NO:304. In embodiments, the full length antibody, F(ab)2 fragment, scFv, or Fab′ fragment includes the sequence of SEQ ID NO:305. In embodiments, the full length antibody, F(ab)2 fragment, scFv, or Fab′ fragment includes the sequence of SEQ ID NO: 306. In embodiments, the full length antibody, F(ab)2 fragment, scFv, or Fab′ fragment includes the sequence of SEQ ID NO:307. In embodiments, the full length antibody, F(ab)2 fragment, scFv, or Fab′ fragment includes the sequence of SEQ ID NO:308. In embodiments, the full length antibody, F(ab)2 fragment, scFv, or Fab′ fragment includes the sequence of SEQ ID NO:309. In embodiments, the full length antibody, F(ab)2 fragment, scFv, or Fab′ fragment includes the sequence of SEQ ID NO:310. In embodiments, the full length antibody, F(ab)2 fragment, scFv, or Fab′ fragment includes the sequence of SEQ ID NO:311. In embodiments, the full length antibody, F(ab)2 fragment, scFv, or Fab′ fragment includes the sequence of SEQ ID NO:312. In embodiments, the full length antibody, F(ab)2 fragment, scFv, or Fab fragment includes the sequence of SEQ ID NO:311 and the sequence of SEQ ID NO:312. In embodiments, at least a portion of the full length antibody, F(ab)2 fragment, scFv, or Fab′ fragment is encoded by the sequence of SEQ ID NO:313. In embodiments, at least a portion of the full length antibody, F(ab)2 fragment, scFv, or Fab′ fragment is encoded by the sequence of SEQ ID NO:314. In embodiments, at least a portion of the full length antibody, F(ab)2 fragment, scFv, or Fab fragment is encoded by the sequence of SEQ ID NO: 315. In embodiments, at least a portion of the full length antibody, F(ab)2 fragment, scFv, or Fab fragment is encoded by the sequence of SEQ ID NO:316. In embodiments, at least a portion of the full length antibody, F(ab)2 fragment, scFv, or Fab fragment is encoded by the sequence of SEQ ID NO:317. In embodiments, at least a portion of the full length antibody, F(ab)2 fragment, scFv, or Fab′ fragment is encoded by the sequence of SEQ ID NO:318. In embodiments, at least a portion of the full length antibody, F(ab)2 fragment, scFv, or Fab′ fragment is encoded by the sequence of SEQ ID NO:319. In embodiments, at least a portion of the full length antibody, F(ab)2 fragment, scFv, or Fab′ fragment is encoded by the sequence of SEQ ID NO:320. In embodiments, a first portion of the full length antibody. F(ab)2 fragment, scFv, or Fab′ fragment is encoded by the sequence of SEQ ID NO:319 and a second portion of the full length antibody, F(ab)2 fragment, scFv, or Fab′ fragment is encoded by the sequence of SEQ ID NO:320. In embodiments, the full length antibody, F(ab)2 fragment, scFv, or Fab′ fragment includes the sequence of SEQ ID NO:321. In embodiments, the full length antibody, F(ab)2 fragment, scFv, or Fab′ fragment includes the sequence of SEQ ID NO:322. In embodiments, the full length antibody, F(ab)2 fragment, scFv, or Fab fragment includes the sequence of SEQ ID NO:323. In embodiments, the full length antibody, F(ab)2 fragment, scFv, or Fab′ fragment includes the sequence of SEQ ID NO:324. In embodiments, the full length antibody, F(ab)2 fragment, scFv, or Fab′ fragment includes the sequence of SEQ ID NO:325. In embodiments, the full length antibody, F(ab)2 fragment, scFv, or Fab fragment includes the sequence of SEQ ID NO:326. In embodiments, the full length antibody, F(ab)2 fragment, scFv, or Fab fragment includes the sequence of SEQ ID NO:327. In embodiments, the full length antibody, F(ab)2 fragment, scFv, or Fab′ fragment includes the sequence of SEQ ID NO:328. In embodiments, the full length antibody, F(ab)2 fragment, scFv, or Fab fragment includes the sequence of SEQ ID NO:327 and the sequence of SEQ ID NO:328. In embodiments, the full length antibody, F(ab)2 fragment, scFv, or Fab′ fragment includes the sequence of SEQ ID NO:329. In embodiments, the full length antibody, F(ab)2 fragment, scFv, or Fab′ fragment includes the sequence of SEQ ID NO:330. In embodiments, the full length antibody, F(ab)2 fragment, scFv, or Fab′ fragment includes the sequence of SEQ ID NO:331. In embodiments, the full length antibody, F(ab)2 fragment, scFv, or Fab fragment includes the sequence of SEQ ID NO:332. In embodiments, the full length antibody, F(ab)2 fragment, scFv, or Fab fragment includes the sequence of SEQ ID NO:333. In embodiments, the full length antibody, F(ab)2 fragment, scFv, or Fab fragment includes the sequence of SEQ ID NO:334. In embodiments, the full length antibody, F(ab)2 fragment, scFv, or Fab fragment includes the sequence of SEQ ID NO:335. In embodiments, the full length antibody, F(ab)2 fragment, scFv, or Fab fragment includes the sequence of SEQ ID NO:336. In embodiments, the full length antibody, F(ab)2 fragment, scFv, or Fab′ fragment includes the sequence of SEQ ID NO:335 and the sequence of SEQ ID NO:336. In embodiments, at least a portion of the full length antibody, F(ab)2 fragment, scFv, or Fab′ fragment is encoded by the sequence of SEQ ID NO:337. In embodiments, at least a portion of the full length antibody, F(ab)2 fragment, scFv, or Fab fragment is encoded by the sequence of SEQ ID NO:338. In embodiments, at least a portion of the full length antibody, F(ab)2 fragment, scFv, or Fab fragment is encoded by the sequence of SEQ ID NO:339. In embodiments, at least a portion of the full length antibody, F(ab)2 fragment, scFv, or Fab fragment is encoded by the sequence of SEQ ID NO:340. In embodiments, at least a portion of the full length antibody, F(ab)2 fragment, scFv, or Fab fragment is encoded by the sequence of SEQ ID NO:341. In embodiments, at least a portion of the full length antibody, F(ab)2 fragment, scFv, or Fab′ fragment is encoded by the sequence of SEQ ID NO:342. In embodiments, at least a portion of the full length antibody, F(ab)2 fragment, scFv, or Fab′ fragment is encoded by the sequence of SEQ ID NO:343. In embodiments, at least a portion of the full length antibody, F(ab)2 fragment, scFv, or Fab fragment is encoded by the sequence of SEQ ID NO:344. In embodiments, a first portion of the full length antibody, F(ab)2 fragment, scFv, or Fab′ fragment is encoded by the sequence of SEQ ID NO:343 and a second portion of the full length antibody, F(ab)2 fragment, scFv, or Fab fragment is encoded by the sequence of SEQ ID NO: 344.

In embodiments, the antibody is an IgG. In embodiments, the antibody is an IgG1. In embodiments, the antibody is a human IgG.

In embodiments, an antibody is capable of binding VISTA or a portion thereof. In embodiments, the antibody is bound to VISTA or a portion thereof. In embodiments, the VISTA forms part of a cell. In embodiments, the cell is a lymphoid cell, a myeloid cell, or a stem cell. In embodiments, the cell is a B cell or a T cell. In embodiments, the cell is a B cell. In embodiments, the cell is a T cell. In embodiments, the antibody is attached to a detectable label.

In one embodiment of a first antibody, the light chain variable domain includes: a CDR L1 as set forth in SEQ ID NO:1, a CDR L2 as set forth in SEQ ID NO:2, a CDR L3 as set forth in SEQ ID NO:3, or a light chain variable domain sequence as set forth in SEQ ID NO:7, and the heavy chain variable domain includes: a CDR H1 as set forth in SEQ ID NO:4, a CDR H2 as set forth in SEQ ID NO: 5, a CDR H3 as set forth in SEQ ID NO:6, or a heavy chain variable domain sequence as set forth in SEQ ID NO:8. In one further embodiment, the first antibody is antibody AT-05.01.

In one embodiment of a first antibody, the light chain variable domain includes: a CDR L1 as set forth in SEQ ID NO:9, a CDR L2 as set forth in SEQ ID NO: 10, a CDR L3 as set forth in SEQ ID NO:11, or a light chain variable domain sequence as set forth in SEQ ID NO:15, and the heavy chain variable domain includes: a CDR H1 as set forth in SEQ ID NO: 12, a CDR H2 as set forth in SEQ ID NO: 13, a CDR H3 as set forth in SEQ ID NO:14, or a heavy chain variable domain sequence as set forth in SEQ ID NO:16. In one further embodiment, the first antibody is antibody AT-05.01v1.

In one embodiment of a first antibody, the light chain variable domain includes: a CDR L1 as set forth in SEQ ID NO: 17, a CDR L2 as set forth in SEQ ID NO:18, a CDR L3 as set forth in SEQ ID NO: 19, or a light chain variable domain sequence as set forth in SEQ ID NO:23, and the heavy chain variable domain includes: a CDR H1 as set forth in SEQ ID NO:20, a CDR H2 as set forth in SEQ ID NO: 21, a CDR H3 as set forth in SEQ ID NO:22, or a heavy chain variable domain sequence as set forth in SEQ ID NO:24. In one further embodiment, the first antibody is antibody AT-05.01v2.

In one embodiment of a first antibody, the light chain variable domain includes: a CDR L1 as set forth in SEQ ID NO:25, a CDR L2 as set forth in SEQ ID NO:26, a CDR L3 as set forth in SEQ ID NO:27, or a light chain variable domain sequence as set forth in SEQ ID NO:31, and the heavy chain variable domain includes: a CDR H1 as set forth in SEQ ID NO:28, a CDR H2 as set forth in SEQ ID NO: 29, a CDR H3 as set forth in SEQ ID NO:30, or a heavy chain variable domain sequence as set forth in SEQ ID NO:32. In one further embodiment, the first antibody is antibody AT-05.01v3.

In one embodiment of a first antibody, the light chain variable domain includes: a CDR L1 as set forth in SEQ ID NO:33, a CDR L2 as set forth in SEQ ID NO:34, a CDR L3 as set forth in SEQ ID NO:35, or a light chain variable domain sequence as set forth in SEQ ID NO:39, and the heavy chain variable domain includes: a CDR H1 as set forth in SEQ ID NO:36, a CDR H2 as set forth in SEQ ID NO: 37, a CDR H3 as set forth in SEQ ID NO:38, or a heavy chain variable domain sequence as set forth in SEQ ID NO:40. In one further embodiment, the first antibody is antibody AT-05.01v4.

In one embodiment of a first antibody, the light chain variable domain includes: a CDR L1 as set forth in SEQ ID NO:41, a CDR L2 as set forth in SEQ ID NO:42, a CDR L3 as set forth in SEQ ID NO:43, or a light chain variable domain sequence as set forth in SEQ ID NO:47, and the heavy chain variable domain includes: a CDR H1 as set forth in SEQ ID NO:44, a CDR H2 as set forth in SEQ ID NO: 45, a CDR H3 as set forth in SEQ ID NO:46, or a heavy chain variable domain sequence as set forth in SEQ ID NO:48. In one further embodiment, the first antibody is antibody AT-05.01v5.

In one embodiment of a first antibody, the light chain variable domain includes: a CDR L1 as set forth in SEQ ID NO:49, a CDR L2 as set forth in SEQ ID NO:50, a CDR L3 as set forth in SEQ ID NO:51, or a light chain variable domain sequence as set forth in SEQ ID NO:55, and the heavy chain variable domain includes: a CDR H1 as set forth in SEQ ID NO:52, a CDR H2 as set forth in SEQ ID NO: 53, a CDR H3 as set forth in SEQ ID NO:54, or a heavy chain variable domain sequence as set forth in SEQ ID NO:56. In one further embodiment, the first antibody is antibody AT-05.01v6.

In one embodiment of a first antibody, the light chain variable domain includes: a CDR L1 as set forth in SEQ ID NO:57, a CDR L2 as set forth in SEQ ID NO:58, a CDR L3 as set forth in SEQ ID NO:59, or a light chain variable domain sequence as set forth in SEQ ID NO:63, and the heavy chain variable domain includes: a CDR H1 as set forth in SEQ ID NO:60, a CDR H2 as set forth in SEQ ID NO: 61, a CDR H3 as set forth in SEQ ID NO:62, or a heavy chain variable domain sequence as set forth in SEQ ID NO:64. In one further embodiment, the first antibody is antibody AT-05.01v7.

In one embodiment of a first antibody, the light chain variable domain includes: a CDR L1 as set forth in SEQ ID NO:65, a CDR L2 as set forth in SEQ ID NO:66, a CDR L3 as set forth in SEQ ID NO:67, or a light chain variable domain sequence as set forth in SEQ ID NO:71, and the heavy chain variable domain includes: a CDR H1 as set forth in SEQ ID NO:68, a CDR H2 as set forth in SEQ ID NO: 69, a CDR H3 as set forth in SEQ ID NO:70, or a heavy chain variable domain sequence as set forth in SEQ ID NO:72. In one further embodiment, the first antibody is antibody AT-05.01v8.

In one embodiment of a first antibody, the light chain variable domain includes: a CDR L1 as set forth in SEQ ID NO:73, a CDR L2 as set forth in SEQ ID NO:74, a CDR L3 as set forth in SEQ ID NO:75, or a light chain variable domain sequence as set forth in SEQ ID NO:79, and the heavy chain variable domain includes: a CDR H1 as set forth in SEQ ID NO:76, a CDR H2 as set forth in SEQ ID NO: 77, a CDR H3 as set forth in SEQ ID NO:78, or a heavy chain variable domain sequence as set forth in SEQ ID NO:80. In one further embodiment, the first antibody is antibody AT-05.01v9.

In one embodiment of a first antibody, the light chain variable domain includes: a CDR L1 as set forth in SEQ ID NO:81, a CDR L2 as set forth in SEQ ID NO:82, a CDR L3 as set forth in SEQ ID NO:83, or a light chain variable domain sequence as set forth in SEQ ID NO:87, and the heavy chain variable domain includes: a CDR H1 as set forth in SEQ ID NO:84, a CDR H2 as set forth in SEQ ID NO: 85, a CDR H3 as set forth in SEQ ID NO:86, or a heavy chain variable domain sequence as set forth in SEQ ID NO:88. In one further embodiment, the first antibody is antibody AT-05.01v10.

In one embodiment of a first antibody, the light chain variable domain includes: a CDR L1 as set forth in SEQ ID NO:89, a CDR L2 as set forth in SEQ ID NO:90, a CDR L3 as set forth in SEQ ID NO:91, or a light chain variable domain sequence as set forth in SEQ ID NO:95, and the heavy chain variable domain includes: a CDR H1 as set forth in SEQ ID NO:92, a CDR H2 as set forth in SEQ ID NO: 93, a CDR H3 as set forth in SEQ ID NO:94, or a heavy chain variable domain sequence as set forth in SEQ ID NO:96. In one further embodiment, the first antibody is antibody AT-05.01v11.

In one embodiment of a first antibody, the light chain variable domain includes: a CDR L1 as set forth in SEQ ID NO:97, a CDR L2 as set forth in SEQ ID NO:98, a CDR L3 as set forth in SEQ ID NO:99, or a light chain variable domain sequence as set forth in SEQ ID NO: 103, and the heavy chain variable domain includes: a CDR H1 as set forth in SEQ ID NO: 100, a CDR H2 as set forth in SEQ ID NO: 101, a CDR H3 as set forth in SEQ ID NO:102, or a heavy chain variable domain sequence as set forth in SEQ ID NO:104. In one further embodiment, the first antibody is antibody AT-05.01v12.

In one embodiment of a first antibody, the light chain variable domain includes: a CDR L1 as set forth in SEQ ID NO:105, a CDR L2 as set forth in SEQ ID NO: 106, a CDR L3 as set forth in SEQ ID NO:107, or a light chain variable domain sequence as set forth in SEQ ID NO: 111, and the heavy chain variable domain includes: a CDR H1 as set forth in SEQ ID NO: 108, a CDR H2 as set forth in SEQ ID NO: 109, a CDR H3 as set forth in SEQ ID NO: 110, or a heavy chain variable domain sequence as set forth in SEQ ID NO:112. In one further embodiment, the first antibody is antibody AT-05.01v13.

In one embodiment of a first antibody, the light chain variable domain includes: a CDR L1 as set forth in SEQ ID NO:113, a CDR L2 as set forth in SEQ ID NO:114, a CDR L3 as set forth in SEQ ID NO:115, or a light chain variable domain sequence as set forth in SEQ ID NO: 119, and the heavy chain variable domain includes: a CDR H1 as set forth in SEQ ID NO: 116, a CDR H2 as set forth in SEQ ID NO:117, a CDR H3 as set forth in SEQ ID NO: 118, or a heavy chain variable domain sequence as set forth in SEQ ID NO: 120. In one further embodiment, the first antibody is antibody AT-05.01v14.

In one embodiment of a first antibody, the light chain variable domain includes: a CDR L1 as set forth in SEQ ID NO: 121, a CDR L2 as set forth in SEQ ID NO:122, a CDR L3 as set forth in SEQ ID NO:123, or a light chain variable domain sequence as set forth in SEQ ID NO: 127, and the heavy chain variable domain includes: a CDR H1 as set forth in SEQ ID NO: 124, a CDR H2 as set forth in SEQ ID NO: 125, a CDR H3 as set forth in SEQ ID NO: 126, or a heavy chain variable domain sequence as set forth in SEQ ID NO: 128. In one further embodiment, the first antibody is antibody AT-05.01v15.

In one embodiment of a first antibody, the light chain variable domain includes: a CDR L1 as set forth in SEQ ID NO: 129, a CDR L2 as set forth in SEQ ID NO: 130, a CDR L3 as set forth in SEQ ID NO: 131, or a light chain variable domain sequence as set forth in SEQ ID NO: 135, and the heavy chain variable domain includes: a CDR H1 as set forth in SEQ ID NO: 132, a CDR H2 as set forth in SEQ ID NO: 133, a CDR H3 as set forth in SEQ ID NO: 134, or a heavy chain variable domain sequence as set forth in SEQ ID NO: 136. In one further embodiment, the first antibody is antibody AT-05.01v16.

In one embodiment of a first antibody, the light chain variable domain includes: a CDR L1 as set forth in SEQ ID NO: 137, a CDR L2 as set forth in SEQ ID NO:138, a CDR L3 as set forth in SEQ ID NO:139, or a light chain variable domain sequence as set forth in SEQ ID NO:143, and the heavy chain variable domain includes: a CDR H1 as set forth in SEQ ID NO: 140, a CDR H2 as set forth in SEQ ID NO:141, a CDR H3 as set forth in SEQ ID NO: 142, or a heavy chain variable domain sequence as set forth in SEQ ID NO:144. In one further embodiment, the first antibody is antibody AT-05.02.

In one embodiment of a first antibody, the light chain variable domain includes: a CDR L1 as set forth in SEQ ID NO: 145, a CDR L2 as set forth in SEQ ID NO: 146, a CDR L3 as set forth in SEQ ID NO: 147, or a light chain variable domain sequence as set forth in SEQ ID NO: 151, and the heavy chain variable domain includes: a CDR H1 as set forth in SEQ ID NO: 148, a CDR H2 as set forth in SEQ ID NO: 149, a CDR H3 as set forth in SEQ ID NO: 150, or a heavy chain variable domain sequence as set forth in SEQ ID NO: 152. In one further embodiment, the first antibody is antibody AT-05.03.

In one embodiment of a first antibody, the light chain variable domain includes: a CDR L1 as set forth in SEQ ID NO:153, a CDR L2 as set forth in SEQ ID NO: 154, a CDR L3 as set forth in SEQ ID NO: 155, or a light chain variable domain sequence as set forth in SEQ ID NO: 159, and the heavy chain variable domain includes: a CDR H1 as set forth in SEQ ID NO: 156, a CDR H2 as set forth in SEQ ID NO: 157, a CDR H3 as set forth in SEQ ID NO: 158, or a heavy chain variable domain sequence as set forth in SEQ ID NO: 160. In one further embodiment, the first antibody is antibody AT-05.04.

In one embodiment of a first antibody, the light chain variable domain includes: a CDR L1 as set forth in SEQ ID NO:161, a CDR L2 as set forth in SEQ ID NO: 162, a CDR L3 as set forth in SEQ ID NO:163, or a light chain variable domain sequence as set forth in SEQ ID NO:167, and the heavy chain variable domain includes: a CDR H1 as set forth in SEQ ID NO: 164, a CDR H2 as set forth in SEQ ID NO:165, a CDR H3 as set forth in SEQ ID NO:166, or a heavy chain variable domain sequence as set forth in SEQ ID NO:168. In one further embodiment, the first antibody is antibody AT-05.05.

In one embodiment of a first antibody, the light chain variable domain includes: a CDR L1 as set forth in SEQ ID NO: 169, a CDR L2 as set forth in SEQ ID NO: 170, a CDR L3 as set forth in SEQ ID NO:171, or a light chain variable domain sequence as set forth in SEQ ID NO: 175, and the heavy chain variable domain includes: a CDR H1 as set forth in SEQ ID NO: 172, a CDR H2 as set forth in SEQ ID NO: 173, a CDR H3 as set forth in SEQ ID NO: 174, or a heavy chain variable domain sequence as set forth in SEQ ID NO:176. In one further embodiment, the first antibody is antibody AT-05.06.

In one embodiment of a first antibody, the light chain variable domain includes: a CDR L1 as set forth in SEQ ID NO:177, a CDR L2 as set forth in SEQ ID NO: 178, a CDR L3 as set forth in SEQ ID NO:179, or a light chain variable domain sequence as set forth in SEQ ID NO: 183, and the heavy chain variable domain includes: a CDR H1 as set forth in SEQ ID NO: 180, a CDR H2 as set forth in SEQ ID NO:181, a CDR H3 as set forth in SEQ ID NO:182, or a heavy chain variable domain sequence as set forth in SEQ ID NO: 184. In one further embodiment, the first antibody is antibody AT-05.07.

In one embodiment of a first antibody, the light chain variable domain includes: a CDR L1 as set forth in SEQ ID NO: 185, a CDR L2 as set forth in SEQ ID NO:186, a CDR L3 as set forth in SEQ ID NO:187, or a light chain variable domain sequence as set forth in SEQ ID NO: 191, and the heavy chain variable domain includes: a CDR H1 as set forth in SEQ ID NO: 188, a CDR H2 as set forth in SEQ ID NO: 189, a CDR H3 as set forth in SEQ ID NO: 190, or a heavy chain variable domain sequence as set forth in SEQ ID NO:192. In one further embodiment, the first antibody is antibody AT-05.08.

In one embodiment of a first antibody, the light chain variable domain includes: a CDR L1 as set forth in SEQ ID NO: 193, a CDR L2 as set forth in SEQ ID NO: 194, a CDR L3 as set forth in SEQ ID NO:195, or a light chain variable domain sequence as set forth in SEQ ID NO: 199, and the heavy chain variable domain includes: a CDR H1 as set forth in SEQ ID NO: 196, a CDR H2 as set forth in SEQ ID NO: 197, a CDR H3 as set forth in SEQ ID NO: 198, or a heavy chain variable domain sequence as set forth in SEQ ID NO:200. In one further embodiment, the first antibody is antibody AT-05.00.

In one embodiment of a first antibody, the light chain variable domain includes: a CDR L1 as set forth in SEQ ID NO:209, a CDR L2 as set forth in SEQ ID NO:210, a CDR L3 as set forth in SEQ ID NO:211, or a light chain variable domain sequence as set forth in SEQ ID NO:215, and the heavy chain variable domain includes: a CDR H1 as set forth in SEQ ID NO:212, a CDR H2 as set forth in SEQ ID NO:213, a CDR H3 as set forth in SEQ ID NO:214, or a heavy chain variable domain sequence as set forth in SEQ ID NO:216. In one further embodiment, the first antibody is antibody AT-05.01v15v1.

In one embodiment of a first antibody, the light chain variable domain includes: a CDR L1 as set forth in SEQ ID NO:217, a CDR L2 as set forth in SEQ ID NO:218, a CDR L3 as set forth in SEQ ID NO:219, or a light chain variable domain sequence as set forth in SEQ ID NO:223, and the heavy chain variable domain includes: a CDR H1 as set forth in SEQ ID NO:220, a CDR H2 as set forth in SEQ ID NO:221, a CDR H3 as set forth in SEQ ID NO:222, or a heavy chain variable domain sequence as set forth in SEQ ID NO:224. In one further embodiment, the first antibody is antibody AT-05.01v15v2.

In one embodiment of a first antibody, the light chain variable domain includes: a CDR L1 as set forth in SEQ ID NO:225, a CDR L2 as set forth in SEQ ID NO:226, a CDR L3 as set forth in SEQ ID NO:227, or a light chain variable domain sequence as set forth in SEQ ID NO:231, and the heavy chain variable domain includes: a CDR H1 as set forth in SEQ ID NO:228, a CDR H2 as set forth in SEQ ID NO:229, a CDR H3 as set forth in SEQ ID NO:230, or a heavy chain variable domain sequence as set forth in SEQ ID NO:232. In one further embodiment, the first antibody is antibody AT-05.01v15v3.

In one embodiment of a first antibody, the light chain variable domain includes: a CDR L1 as set forth in SEQ ID NO:233, a CDR L2 as set forth in SEQ ID NO:234, a CDR L3 as set forth in SEQ ID NO:235, or a light chain variable domain sequence as set forth in SEQ ID NO:239, and the heavy chain variable domain includes: a CDR H1 as set forth in SEQ ID NO:236, a CDR H2 as set forth in SEQ ID NO:237, a CDR H3 as set forth in SEQ ID NO:238, or a heavy chain variable domain sequence as set forth in SEQ ID NO:240. In one further embodiment, the first antibody is antibody AT-05.01v15v4.

In one embodiment of a first antibody, the light chain variable domain includes: a CDR L1 as set forth in SEQ ID NO:241, a CDR L2 as set forth in SEQ ID NO:242, a CDR L3 as set forth in SEQ ID NO:243, or a light chain variable domain sequence as set forth in SEQ ID NO:247, and the heavy chain variable domain includes: a CDR H1 as set forth in SEQ ID NO:244, a CDR H2 as set forth in SEQ ID NO:245, a CDR H3 as set forth in SEQ ID NO:246, or a heavy chain variable domain sequence as set forth in SEQ ID NO:248. In one further embodiment, the first antibody is antibody AT-05.01v15v5.

In one embodiment of a first antibody, the light chain variable domain includes: a CDR L1 as set forth in SEQ ID NO:249, a CDR L2 as set forth in SEQ ID NO:250, a CDR L3 as set forth in SEQ ID NO:251, or a light chain variable domain sequence as set forth in SEQ ID NO:255, and the heavy chain variable domain includes: a CDR H1 as set forth in SEQ ID NO:252, a CDR H2 as set forth in SEQ ID NO:253, a CDR H3 as set forth in SEQ ID NO:254, or a heavy chain variable domain sequence as set forth in SEQ ID NO:256. In one further embodiment, the first antibody is antibody AT-05.01v15v6.

In one embodiment of a first antibody, the light chain variable domain includes: a CDR L1 as set forth in SEQ ID NO:257, a CDR L2 as set forth in SEQ ID NO:258, a CDR L3 as set forth in SEQ ID NO:259, or a light chain variable domain sequence as set forth in SEQ ID NO:263, and the heavy chain variable domain includes: a CDR H1 as set forth in SEQ ID NO:260, a CDR H2 as set forth in SEQ ID NO:261, a CDR H3 as set forth in SEQ ID NO:262, or a heavy chain variable domain sequence as set forth in SEQ ID NO:264. In one further embodiment, the first antibody is antibody AT-05.01v15v7.

In one embodiment of a first antibody, the light chain variable domain includes: a CDR L1 as set forth in SEQ ID NO:265, a CDR L2 as set forth in SEQ ID NO:266, a CDR L3 as set forth in SEQ ID NO:267, or a light chain variable domain sequence as set forth in SEQ ID NO:271, and the heavy chain variable domain includes: a CDR H1 as set forth in SEQ ID NO:268, a CDR H2 as set forth in SEQ ID NO:269, a CDR H3 as set forth in SEQ ID NO:270, or a heavy chain variable domain sequence as set forth in SEQ ID NO:272. In one further embodiment, the first antibody is antibody AT-05.01v15v8.

In one embodiment of a first antibody, the light chain variable domain includes: a CDR L1 as set forth in SEQ ID NO:273, a CDR L2 as set forth in SEQ ID NO:274, a CDR L3 as set forth in SEQ ID NO:275, or a light chain variable domain sequence as set forth in SEQ ID NO:279, and the heavy chain variable domain includes: a CDR H1 as set forth in SEQ ID NO:276, a CDR H2 as set forth in SEQ ID NO:277, a CDR H3 as set forth in SEQ ID NO:278, or a heavy chain variable domain sequence as set forth in SEQ ID NO:280. In one further embodiment, the first antibody is antibody AT-05.01v15v9.

In one embodiment of a first antibody, the light chain variable domain includes: a CDR L1 as set forth in SEQ ID NO:281, a CDR L2 as set forth in SEQ ID NO:282, a CDR L3 as set forth in SEQ ID NO:283, or a light chain variable domain sequence as set forth in SEQ ID NO:287, and the heavy chain variable domain includes: a CDR H1 as set forth in SEQ ID NO:284, a CDR H2 as set forth in SEQ ID NO:285, a CDR H3 as set forth in SEQ ID NO:286, or a heavy chain variable domain sequence as set forth in SEQ ID NO:288. In one further embodiment, the first antibody is antibody AT-05.01v15v10.

In one embodiment of a first antibody, the light chain variable domain includes: a CDR L1 as set forth in SEQ ID NO:289, a CDR L2 as set forth in SEQ ID NO:290, a CDR L3 as set forth in SEQ ID NO:291, or a light chain variable domain sequence as set forth in SEQ ID NO:295, and the heavy chain variable domain includes: a CDR H1 as set forth in SEQ ID NO:292, a CDR H2 as set forth in SEQ ID NO:293, a CDR H3 as set forth in SEQ ID NO:294, or a heavy chain variable domain sequence as set forth in SEQ ID NO:296. In one further embodiment, the first antibody is antibody AT-05.01v15v11.

In one embodiment of a first antibody, the light chain variable domain includes: a CDR L1 as set forth in SEQ ID NO:297, a CDR L2 as set forth in SEQ ID NO:298, a CDR L3 as set forth in SEQ ID NO:299, or a light chain variable domain sequence as set forth in SEQ ID NO:303, and the heavy chain variable domain includes: a CDR H1 as set forth in SEQ ID NO:300, a CDR H2 as set forth in SEQ ID NO:301, a CDR H3 as set forth in SEQ ID NO:302, or a heavy chain variable domain sequence as set forth in SEQ ID NO:304. In one further embodiment, the first antibody is antibody AT-05.01v15v12.

In one embodiment of a first antibody, the light chain variable domain includes: a CDR L1 as set forth in SEQ ID NO:305, a CDR L2 as set forth in SEQ ID NO:306, a CDR L3 as set forth in SEQ ID NO:307, or a light chain variable domain sequence as set forth in SEQ ID NO:311, and the heavy chain variable domain includes: a CDR H1 as set forth in SEQ ID NO:308, a CDR H2 as set forth in SEQ ID NO:309, a CDR H3 as set forth in SEQ ID NO:310, or a heavy chain variable domain sequence as set forth in SEQ ID NO:312. In one further embodiment, the first antibody is antibody AT-05.01v15v13.

In one embodiment of a first antibody, the light chain variable domain includes: a CDR L1 as set forth in SEQ ID NO:313, a CDR L2 as set forth in SEQ ID NO:314, a CDR L3 as set forth in SEQ ID NO:315, or a light chain variable domain sequence as set forth in SEQ ID NO:319, and the heavy chain variable domain includes: a CDR H1 as set forth in SEQ ID NO:316, a CDR H2 as set forth in SEQ ID NO:317, a CDR H3 as set forth in SEQ ID NO:318, or a heavy chain variable domain sequence as set forth in SEQ ID NO:320. In one further embodiment, the first antibody is antibody AT-05.01v15v14.

In one embodiment of a first antibody, the light chain variable domain includes: a CDR L1 as set forth in SEQ ID NO:321, a CDR L2 as set forth in SEQ ID NO:322, a CDR L3 as set forth in SEQ ID NO:323, or a light chain variable domain sequence as set forth in SEQ ID NO:327, and the heavy chain variable domain includes: a CDR H1 as set forth in SEQ ID NO:324, a CDR H2 as set forth in SEQ ID NO:325, a CDR H3 as set forth in SEQ ID NO:326, or a heavy chain variable domain sequence as set forth in SEQ ID NO:328. In one further embodiment, the first antibody is antibody AT-05.01v15v15.

In one embodiment of a first antibody, the light chain variable domain includes: a CDR L1 as set forth in SEQ ID NO:329, a CDR L2 as set forth in SEQ ID NO:330, a CDR L3 as set forth in SEQ ID NO:331, or a light chain variable domain sequence as set forth in SEQ ID NO:335, and the heavy chain variable domain includes: a CDR H1 as set forth in SEQ ID NO:332, a CDR H2 as set forth in SEQ ID NO:333, a CDR H3 as set forth in SEQ ID NO:334, or a heavy chain variable domain sequence as set forth in SEQ ID NO:336. In one further embodiment, the first antibody is antibody AT-05.01v15v16.

In one embodiment of a first antibody, the light chain variable domain includes: a CDR L1 as set forth in SEQ ID NO:337, a CDR L2 as set forth in SEQ ID NO:338, a CDR L3 as set forth in SEQ ID NO:339, or a light chain variable domain sequence as set forth in SEQ ID NO:343, and the heavy chain variable domain includes: a CDR H1 as set forth in SEQ ID NO:340, a CDR H2 as set forth in SEQ ID NO:341, a CDR H3 as set forth in SEQ ID NO:342, or a heavy chain variable domain sequence as set forth in SEQ ID NO:344. In one further embodiment, the first antibody is antibody AT-05.01v15v17.

### Recombinant Protein Compositions

As described above, the light chain variable (VL) domain and the heavy chain variable (VH) domain provided herein including embodiments thereof, may each independently form part of an antibody, an antibody variant, a fragment of an antibody, a fragment of an antibody variant, or a recombinant protein (e.g., an scFv antibody, a bispecific antibody). Provided herein are, inter alia, recombinant proteins (e.g., scFv antibodies), which include the light chain variable (VL) domain and/or the heavy chain variable (VH) domain as provided herein and are therefore capable of binding VISTA, thereby effectively inhibiting VISTA activity. In embodiments, the recombinant protein is a scFv. In embodiments, the recombinant protein is a bispecific antibody.

### Bispecific Antibodies

The light chain variable (VL) domain and the heavy chain variable (VH) domain as provided herein may form part of a bispecific antibody. Thus, the second antibody region may include any of the light chain and/or heavy chain variable domains provided herein including embodiments thereof.

The recombinant proteins (e.g., scFv or bispecific antibody) provided herein may include any of the VISTA antibodies or fragments thereof described herein. Thus, the recombinant protein (e.g., scFv or bispecific antibody) may include any of the CDRs, heavy chain variable domains, or light chain variable domains provided herein.

The heavy chain variable domain of the recombinant protein (e.g., bispecific antibody) provided herein may include any of the CDRs or variable domains provided herein. The light chain variable domain of the recombinant protein (e.g., bispecific antibody) provided herein may include any of the CDRs or variable domains provided herein.

In embodiments, the first antibody region is a first Fab′ fragment or the second antibody region is a second Fab′ fragment. In embodiments, the first antibody region is a single chain variable fragment (scFv) or the second antibody region is a second single chain variable fragment (scFv).

In embodiments, a bispecific antibody can comprise a light chain variable (VL) domain and/or a heavy chain variable (VH) domain capable of (e.g., specifically) associating with or binding to a CD3 transmembrane protein (e.g., CD3-zeta) of an immune cell, such as a T-cell. In some cases, the bispecific antibody can also comprise a light chain variable (VL) domain and/or heavy chain variable (VH) domain capable of associating with or binding to an antigen associated with tumor or cancer cell function or viability (e.g., VISTA). For example, a first antibody region of a bispecific antibody may include a light chain variable (VL) domain (or portion thereof, such as a LCDR1, LCDR2, and LCDR3) and a heavy chain variable (VH) domain (or portion thereof, such as a HCDR1, HCDR2, and HCDR3) capable of specifically recognizing (e.g., specifically associating with or binding to) CD3 or a portion thereof, and a second antibody region of the bispecific antibody having a light chain variable (VL) domain (or portion thereof, such as a LCDR1, LCDR2, and LCDR3) and a heavy chain variable (VH) domain (or portion thereof, such as a HCDR1, HCDR2, and HCDR3) capable of specifically recognizing (e.g., specifically associating with or binding to) an antigen associated with tumor or cancer cell function or viability (e.g., VISTA). In some cases, inclusion of an VL and/or VH capable of associating with or binding to CD3 improve a treatment of a tumor or cancer (e.g., by activating an immune cell, such as a T-cell, of the organism having the tumor or cancer).

### Nucleic Acid Compositions

Disclosed herein are nucleic acid sequences encoding polypeptides, or portions thereof, useful in the treatment of a subject (e.g., a subject having, at risk of having, or suspected of having a tumor or cancer, such as a solid tumor or cancer). In some cases, a nucleic acid sequence described herein can encode one or more amino acid sequences described herein, such as one or more amino acid sequences as set forth in Table 5. In some cases, a nucleic acid sequence disclosed herein can encode an AT-05 antibody disclosed herein. The compositions provided herein can include nucleic acid molecules encoding one or more anti-VISTA antibodies (e.g., a AT-05 antibody comprising one or more sequences set forth in Table 5) and recombinant proteins provided herein including embodiments thereof. Thus, in an aspect, an isolated nucleic acid encoding an antibody as provided herein including embodiments thereof is provided. In another aspect, an isolated nucleic acid encoding a recombinant protein as provided herein, including embodiments thereof, is provided.

In some cases, a nucleic acid comprising one more amino acid sequences disclosed herein (e.g., such that the nucleic acid encodes an AT-05 antibody described herein) can be administered to a subject in need of treatment (e.g., in a composition, which may comprise a pharmaceutically acceptable excipient), for example, wherein the subject has, is at risk of having, or is suspected of having a tumor or cancer (e.g., a solid tumor or cancer), as described herein.

### Cells

The antibodies provided herein may be used as immunotherapeutic agents. In an aspect a cell including an antibody provided herein including embodiments thereof is provided. In another aspect, a nucleic acid encoding an antibody provided herein including embodiments thereof is provided. In embodiments, the cell can be a lymphoid cell, a myeloid cell, or a stem cell. In embodiments, the (e.g., lymphoid) cell is a T cell or a B cell. In embodiments, the cell is a T cell. In embodiments, the cell is a B cell. In embodiments, the cell is a VISTA (e.g., high) expressing cell. In some aspects, a cell can comprise an isolated nucleic acid encoding a (e.g., recombinant) protein as provided herein, for example, wherein the protein is an antibody or fragment thereof described herein, such as an anti-VISTA antibody.

### Pharmaceutical Compositions

The compositions provided herein include pharmaceutical compositions including the anti-VISTA antibody provided herein including embodiments thereof. Thus, in an aspect is provided a pharmaceutical composition including a therapeutically effective amount of an antibody provided herein including embodiments thereof and a pharmaceutically acceptable excipient.

In some cases, a composition described herein comprising an anti-VISTA antibody described herein or nucleic acid comprising an anti-VISTA antibody described herein can be packaged with or combined into a single formulation with an additional pharmaceutical agent. For example, an anti-VISTA antibody described herein (e.g., an antibody comprising one or more amino acid sequences shown in Table 5, for example, such that the anti-VISTA antibody is an AT-05 antibody family member, such as AT-05.01, AT-05.01v1, AT-05.01v2, AT-05.01v3, AT-05.01v4, AT-05.01v5, AT-05.01v6, AT-05.01v7, AT-05.01v8, AT-05.01v9, AT-05.01v10, AT-05.01v11, AT-05.01v12, AT-05.01v13, AT-05.01v14, AT-05.01v15, AT-05.01v15v1, AT-05.01v15v2, AT-05.01v15v3, AT-05.01v15v4, AT-05.01v15v5, AT-05.01v15v6, AT-05.01v15v7, AT-05.01v15v8, AT-05.01v15v9, AT-05.01v15v10, AT-05.01v15v11, AT-05.01v15v12, AT-05.01v15v13, AT-05.01v15v14, AT-05.01v15v15, AT-05.01v15v16, AT-05.01v15v17, AT-05.01v16, AT-05.02, AT-05.03, AT-05.04, AT-05.05, AT-05.06, AT-05.07, AT-05.08, or AT-05.00) can be formulated or packaged with an additional pharmaceutical agent. In some cases, the additional pharmaceutical agent can be a checkpoint inhibitor. In some cases, the additional pharmaceutical agent can be a T-cell checkpoint inhibitor. In some cases, the additional pharmaceutical agent can be a PD-1 pathway inhibitor. In some cases, the additional pharmaceutical agent can be selected from a PD-1 inhibitor or a PD-L1 inhibitor. In some cases, the additional pharmaceutical agent can be pembrolizumab (Keytruda®) or a derivative thereof. In some cases, the additional pharmaceutical agent can be nivolumab (Opdivo®) or a derivative thereof. In some cases, the additional pharmaceutical agent can be cemiplimab (Libtayo®) or a derivative thereof. In some cases, the additional pharmaceutical agent can be tislelizumab (Tevimbra®) or a derivative thereof. In some cases, the additional pharmaceutical agent can be atezolizumab (TECENTRIQ®) or a derivative thereof. In some cases, the additional pharmaceutical agent can be durvalumab (IMFINZI®) or a derivative thereof. In some cases, the additional pharmaceutical agent can be avelumab (BAVENCIO®) or a derivative thereof. In some cases, the additional pharmaceutical agent can be dostarlimab (JEMPERLI®) or a derivative thereof. In some cases, the additional pharmaceutical agent can be retifanlimab (Zynyz®) or a derivative thereof. In some cases, the additional pharmaceutical agent can be toripalimab (Loqtorzi®) or a derivative thereof.

### Methods

The compositions (e.g., anti-VISTA antibodies and recombinant proteins) provided herein, including embodiments thereof, are contemplated as providing effective treatments for diseases such as a tumor or cancer (e.g., a solid tumor or cancer, such as a colorectal cancer). Thus, in an aspect is provided a method of treating cancer (e.g., a solid tumor or cancer, such as a colorectal cancer) in a subject in need thereof, the method including administering to a subject a therapeutically effective amount of an antibody as provided herein including embodiments thereof, thereby treating cancer in the subject.

In another aspect is provided a method of treating a tumor or cancer (e.g., a solid tumor or cancer, such as a colorectal cancer) in a subject in need thereof, the method including administering to a subject a therapeutically effective amount of a recombinant protein as described herein, including embodiments thereof, thereby treating cancer in the subject. In some aspects, a composition described herein (e.g., an anti-VISTA antibody and recombinant proteins described herein) can be used to treat a plurality of cancer types.

In some cases, a method of treating a subject can comprise identifying a subject in need thereof (e.g., a subject having, considered at risk of having, or suspected of having a tumor or cancer, such as a solid tumor or cancer). In some cases, a subject can be considered at risk of having or suspected of having a cancer (e.g., a solid cancer, such as a colorectal cancer) can have one or more mutations known or thought to cause a tumor or cancer. In some cases, a subject having, considered at risk of having, or suspected of having a tumor or cancer (e.g., a solid cancer, such as a colorectal cancer) can be treated using a regimen comprising administration of a composition comprising an AT-05 antibody described herein (e.g., an AT-05.01, AT-05.01v1, AT-05.01v2, AT-05.01v3, AT-05.01v4, AT-05.01v5, AT-05.01v6, AT-05.01v7, AT-05.01v8, AT-05.01v9, AT-05.01v10, AT-05.01v11, AT-05.01v12, AT-05.01v13, AT-05.01v14, AT-05.01v15, AT-05.01v15v1. AT-05.01v15v2. AT-05.01v15v3, AT-05.01v15v4, AT-05.01v15v5, AT-05.01v15v6, AT-05.01v15v7, AT-05.01v15v8, AT-05.01v15v9, AT-05.01v15v10, AT-05.01v15v11. AT-05.01v15v12. AT-05.01v15v13, AT-05.01v15v14, AT-05.01v15v15, AT-05.01v15v16, AT-05.01v15v17. AT-05.01v16, AT-05.02, AT-05.03, AT-05.04, AT-05.05, AT-05.06, AT-05.07, AT-05.08, or AT-05.00 antibody, for example, wherein the antibody comprises one or more sequences as set forth in Table 5). In some cases, a subject having, considered at risk of having, or suspected of having a cancer or tumor (e.g., a solid cancer, such as a colorectal cancer) can be treated using a regimen comprising administration of a composition comprising an AT-05 antibody described herein and a composition comprising one or more PD-1 pathway inhibitors (e.g., pembrolizumab (Keytruda®) or a derivative thereof, nivolumab (Opdivo®) or a derivative thereof, cemiplimab (Libtayo®) or a derivative thereof, tislelizumab (Tevimbra®) or a derivative thereof, atezolizumab (TECENTRIQ®) or a derivative thereof, durvalumab (IMFINZI®) or a derivative thereof, avelumab (BAVENCIO®) or a derivative thereof, dostarlimab (JEMPERLI®) or a derivative thereof, retifanlimab (Zynyz®) or a derivative thereof, toripalimab (Loqtorzi®) or a derivative thereof). In some cases, the composition comprising the AT-05 antibody and the composition comprising the PD-1 pathway inhibitor can be administered to the subject at the same time (e.g., wherein the AT-05 antibody and the PD-1 pathway inhibitor are present in the same composition). In some cases, the composition comprising the AT-05 antibody and the composition comprising the PD-1 pathway inhibitor can be administered to the subject at different times. A method can comprise administering to a subject in need of treatment a composition comprising an AT-05 antibody (e.g., an AT-05.01, AT-05.01v1, AT-05.01v2, AT-05.01v3, AT-05.01v4, AT-05.01v5, AT-05.01v6, AT-05.01v7, AT-05.01v8, AT-05.01v9, AT-05.01v10, AT-05.01v11, AT-05.01v12, AT-05.01v13, AT-05.01v14, AT-05.01v15, AT-05.01v15v1, AT-05.01v15v2, AT-05.01v15v3, AT-05.01v15v4, AT-05.01v15v5, AT-05.01v15v6, AT-05.01v15v7, AT-05.01v15v8, AT-05.01v15v9, AT-05.01v15v10, AT-05.01v15v11. AT-05.01v15v12, AT-05.01v15v13, AT-05.01v15v14, AT-05.01v15v15, AT-05.01v15v16, AT-05.01v15v17. AT-05.01v16, AT-05.02, AT-05.03, AT-05.04, AT-05.05, AT-05.06, AT-05.07, AT-05.08, or AT-05.00 antibody, for example, wherein the antibody comprises one or more sequences as set forth in Table 5). The method can further comprise administering to a subject in need of treatment (e.g., a subject having, at risk of having, or suspected of having a tumor or cancer, such as a solid tumor or cancer) a composition comprising the AT-05 antibody and a composition comprising a PD-1 pathway inhibitor (e.g., wherein the composition comprising the AT-05 antibody is the same composition as the composition comprising the PD-1 pathway inhibitor or wherein the composition comprising the AT-05 antibody is a different composition than the composition comprising the PD-1 pathway inhibitor).

In some cases, a tumor or cancer treated with a composition described herein can be a solid tumor or solid cancer. In embodiments, the cancer is lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, renal cancer, melanoma, breast cancer, neuroblastoma, or glioblastoma. In embodiments, the cancer is melanoma. In embodiments, the cancer is breast cancer. In embodiments, the cancer is pancreatic cancer. In embodiments, the cancer is lung cancer. In embodiments, the lung cancer is non-small cell lung cancer. In embodiments, the cancer is ovarian cancer. In embodiments, the cancer is a glioblastoma. In embodiments, the cancer is colorectal cancer. In embodiments, the cancer is renal cancer.

In some cases, an anti-VISTA antibody can be useful in the treatment of one or more cancers or tumors selected from one or more of: lung cancer (e.g., non-small cell lung cancer), breast cancer, ovarian cancer, renal cancer, glioblastoma, pancreatic cancer, and/or colorectal cancer. In some cases, an anti-VISTA antibody can be useful in reducing adhesion of cells from one or more of: a lung cancer (e.g., non-small cell lung cancer), a breast cancer, an ovarian cancer, a renal cancer, glioblastoma, pancreatic cancer, and/or a colorectal cancer (e.g., compared to a control treatment, which may include a vehicle control, a placebo control, or no treatment). In some cases, an anti-VISTA antibody can be useful in reducing proliferation of cells from one or more of: a lung cancer (e.g., non-small cell lung cancer), a breast cancer, an ovarian cancer, a renal cancer, glioblastoma, pancreatic cancer, and/or a colorectal cancer (e.g., compared to a control treatment, which may include a vehicle control, a placebo control, or no treatment). In some cases, an anti-VISTA antibody can be useful in inhibiting migration (or metastasis) of cells from one or more of: a lung cancer (e.g., non-small cell lung cancer), a breast cancer, an ovarian cancer, a renal cancer, glioblastoma, pancreatic cancer, and/or a colorectal cancer (e.g., compared to a control treatment, which may include a vehicle control, a placebo control, or no treatment). In some cases, an anti-VISTA antibody can be useful in decreasing secretion of an interleukin (e.g., interleukin-6) in a cell from one or more of: a lung cancer (e.g., non-small cell lung cancer), a breast cancer, an ovarian cancer, a renal cancer, glioblastoma, pancreatic cancer, and/or a colorectal cancer (e.g., compared to a control treatment, which may include a vehicle control, a placebo control, or no treatment). In some cases, an anti-VISTA antibody can be useful in increasing peripheral mononuclear blood cell viability in a subject having one or more of: a lung cancer (e.g., non-small cell lung cancer), a breast cancer, an ovarian cancer, a renal cancer, glioblastoma, pancreatic cancer, and/or a colorectal cancer (e.g., compared to a control treatment, which may include a vehicle control, a placebo control, or no treatment). In some cases, an anti-S100A8 antibody can modulate (e.g., endogenous) VISTA signaling, which may have the effect of abrogating or eliminating endogenous immune cell-mediated reductions in tumor or cancer cell proliferation, migration, or pro-inflammatory secretion (e.g., as compared to control treatment, which may include a vehicle control, a placebo control, or no treatment).

In embodiments, the method further includes administering to the subject a second therapeutic agent. In embodiments, the method further includes administering a therapeutically effective amount of an antineoplastic agent. In some cases, the second therapeutic agent can comprise one or more PD-1 pathway inhibitors (e.g., pembrolizumab (Keytruda®) or a derivative thereof, nivolumab (Opdivo®) or a derivative thereof, cemiplimab (Libtayo®) or a derivative thereof, tislelizumab (Tevimbra®) or a derivative thereof, atezolizumab (TECENTRIQ®) or a derivative thereof, durvalumab (IMFINZI®) or a derivative thereof, avelumab (BAVENCIO®) or a derivative thereof, dostarlimab (JEMPERLI®) or a derivative thereof, retifanlimab (Zynyz®) or a derivative thereof, toripalimab (Loqtorzi®) or a derivative thereof). In embodiments, the effective amount of an antibody and the effective amount of an antineoplastic agent are a combined synergistic amount. In embodiments, an antineoplastic agent can comprise a chemotherapeutic drug.

In embodiments, the antibody and the antineoplastic agent are administered sequentially or concurrently. In embodiments, the antibody and the antineoplastic agent are administered sequentially. In embodiments, the antibody and the antineoplastic agent are administered concurrently. In embodiments, the antibody and the antineoplastic agent are admixed together prior to administration. In embodiments, the antibody and the antineoplastic agent are administered in a single dosage form. In embodiments, the antibody and the antineoplastic agent are administered in two separate dosage forms.

In an aspect is provided a method of treating cancer in a subject in need thereof, the method including administering to a subject a combined effective amount of an antibody as provided herein including embodiments thereof and a antineoplastic agent, thereby treating cancer in the subject. In embodiments, the combined effective amount is a combined synergistic amount.

In embodiments, the effective amount of an antibody provided herein including embodiments thereof, and the effective amount of an antineoplastic agent are a combined synergistic amount. A “combined synergistic amount” as used herein refers to the sum of a first amount (e.g., an amount of an antibody provided herein including embodiments thereof) and a second amount (e.g., an amount of an antineoplastic agent), that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of compounds administered in combination where the measured effect is greater than the sum of the individual effects of each of the compounds administered alone as a single agent. In embodiments, the measured effect of the compounds (e.g., the antibody provided herein and the antineoplastic agent) administered is 1-fold, 1.5-fold, 2-fold, 3-fold, 4-fold, 5-fold, 10-fold or greater than the sum of the individual effects of each of the compounds administered alone as a single agent.

In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the antibody provided herein when used separately from the antineoplastic agent. In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the antineoplastic agent when used separately from the antibody provided herein.

In embodiments, the antibody is administered intratumorally, intravenously, subcutaneously, intraperitoneally, intradermally, or intramuscularly (e.g., in pharmaceutically acceptable formulation).

In embodiments, the antibody is administered at an amount from about 10 micrograms per deciliter (ug/dL) to about 2000 ug/dL. In embodiments, the antibody is administered at an amount from about 10 ug/dL to about 1500 ug/dL, from about 10 ug/dL to about 1000 ug/dL, from about 10 ug/dL to about 900 ug/dL, from about 10 ug/dL to about 800 ug/dL, from about 10 ug/dL to about 700 ug/dL, from about 10 ug/dL to about 600 ug/dL, from about 10 ug/dL to about 500 ug/dL, from about 10 ug/dL to about 400 ug/dL, from about 10 ug/dL to about 300 ug/dL, from about 10 ug/dL to about 200 ug/dL, from about 10 ug/dL to about 100 ug/dL, from about 10 ug/dL to about 50 ug/dL, from about 50 ug/dL to about 2000 ug/dL, from about 50 ug/dL to about 1500 ug/dL, from about 50 ug/dL to about 1000 ug/dL, from about 50 ug/dL to about 900 ug/dL, from about 50 ug/dL to about 800 ug/dL, from about 50 ug/dL to about 700 ug/dL, from about 50 ug/dL to about 600 ug/dL, from about 50 ug/dL to about 500 ug/dL, from about 50 ug/dL to about 400 ug/dL, from about 50 ug/dL to about 300 ug/dL, from about 50 ug/dL to about 200 ug/dL, from about 50 ug/dL to about 100 ug/dL, from about 100 ug/dL to about 2000 ug/dL, from about 100 ug/dL to about 1500 ug/dL, from about 100 ug/dL to about 1000 ug/dL, from about 100 ug/dL to about 900 ug/dL, from about 100 ug/dL to about 800 ug/dL, from about 100 ug/dL to about 700 ug/dL, from about 100 ug/dL to about 600 ug/dL, from about 100 ug/dL to about 500 ug/dL, from about 100 ug/dL to about 400 ug/dL, from about 100 ug/dL to about 300 ug/dL, from about 100 ug/dL to about 200 ug/dL, from about 200 ug/dL to about 2000 ug/dL, from about 200 ug/dL to about 1500 ug/dL, from about 200 ug/dL to about 1000 ug/dL, from about 200 ug/dL to about 900 ug/dL, from about 200 ug/dL to about 800 ug/dL, from about 200 ug/dL to about 700 ug/dL, from about 200 ug/dL to about 600 ug/dL, from about 200 ug/dL to about 500 ug/dL, from about 200 ug/dL to about 400 ug/dL, from about 200 ug/dL to about 300 ug/dL, from about 300 ug/dL to about 2000 ug/dL, from about 300 ug/dL to about 1500 ug/dL, from about 300 ug/dL to about 1000 ug/dL, from about 300 ug/dL to about 900 ug/dL, from about 300 ug/dL to about 800 ug/dL, from about 300 ug/dL to about 700 ug/dL, from about 300 ug/dL to about 600 ug/dL, from about 300 ug/dL to about 500 ug/dL, from about 300 ug/dL to about 400 ug/dL, from about 400 ug/dL to about 2000 ug/dL, from about 400 ug/dL to about 1500 ug/dL, from about 400 ug/dL to about 1000 ug/dL, from about 400 ug/dL to about 900 ug/dL, from about 400 ug/dL to about 800 ug/dL, from about 400 ug/dL to about 700 ug/dL, from about 400 ug/dL to about 600 ug/dL, from about 400 ug/dL to about 500 ug/dL, from about 500 ug/dL to about 2000 ug/dL, from about 500 ug/dL to about 1500 ug/dL, from about 500 ug/dL to about 1000 ug/dL, from about 500 ug/dL to about 900 ug/dL, from about 500 ug/dL to about 800 ug/dL, from about 500 ug/dL to about 700 ug/dL, from about 500 ug/dL to about 600 ug/dL, from about 600 ug/dL to about 2000 ug/dL, from about 600 ug/dL to about 1500 ug/dL, from about 600 ug/dL to about 1000 ug/dL, from about 600 ug/dL to about 900 ug/dL, from about 600 ug/dL to about 800 ug/dL, from about 600 ug/dL to about 700 ug/dL, from about 700 ug/dL to about 2000 ug/dL, from about 700 ug/dL to about 1500 ug/dL, from about 700 ug/dL to about 1000 ug/dL, from about 700 ug/dL to about 900 ug/dL, from about 700 ug/dL to about 800 ug/dL, from about 800 ug/dL to about 2000 ug/dL, from about 800 ug/dL to about 1500 ug/dL, from about 800 ug/dL to about 1000 ug/dL, from about 800 ug/dL to about 900 ug/dL, from about 900 ug/dL to about 2000 ug/dL, from about 900 ug/dL to about 1500 ug/dL, from about 900 ug/dL to about 1000 ug/dL, from about 1000 ug/dL to about 2000 ug/dL, from about 1000 ug/dL to about 1500 ug/dL, or from about 1500 ug/dL to about 2000 ug/dL. In embodiments, the antibody is administered at an amount of at least about 10 ug/dL, at least about 50 ug/dL, at least about 100 ug/dL, at least about 200 ug/dL, at least about 300 ug/dL, at least about 400 ug/dL, at least about 500 ug/dL, at least about 600 ug/dL, at least about 700 ug/dL, at least about 800 ug/dL, at least about 900 ug/dL, at least about 1000 ug/dL, at least about 1500 ug/dL, or at least about 2000 ug/dL. In embodiments, the antibody is administered at an amount of at most about 10 ug/dL, at most about 50 ug/dL, at most about 100 ug/dL, at most about 200 ug/dL, at most about 300 ug/dL, at most about 400 ug/dL, at most about 500 ug/dL, at most about 600 ug/dL, at most about 700 ug/dL, at most about 800 ug/dL, at most about 900 ug/dL, at most about 1000 ug/dL, at most about 1500 ug/dL, or at most about 2000 ug/dL. In embodiments, the antibody is administered at an amount of about 10 ug/dL, about 50 ug/dL, about 100 ug/dL, about 200 ug/dL, about 300 ug/dL, about 400 ug/dL, about 500 ug/dL, about 600 ug/dL, about 700 ug/dL, about 800 ug/dL, about 900 ug/dL, about 1000 ug/dL, about 1500 ug/dL, or about 2000 ug/dL.

In embodiments, the antibody is administered at an amount from about 0.01 nM to about 10 nM. In embodiments, the antibody is administered at an amount from about 0.05 nM to about 10 nM. In embodiments, the antibody is administered at an amount from about 0.1 nM to about 10 nM. In embodiments, the antibody is administered at an amount from about 0.5 nM to about 10 nM. In embodiments, the antibody is administered at an amount from about 1 nM to about 10 nM. In embodiments, the antibody is administered at an amount from about 2 nM to about 10 nM. In embodiments, the antibody is administered at an amount from about 4 nM to about 10 nM. In embodiments, the antibody is administered at an amount from about 6 nM to about 10 nM. In embodiments, the antibody is administered at an amount from about 4 nM to about 10 nM. In embodiments, the antibody is administered at an amount from about 8 nM to about 10 nM. In embodiments, the antibody is administered at an amount of about 0.01 nM, 0.05 nM. 0.1 nM, 0.5 nM, 1 nM, 2 nM, 2 nM, 4 nM, 5 nM, 6 nM, 7 nM, 8 nM, 9 nM or 10 nM.

In embodiments, the antibody is administered at an amount from 0.01 nM to 10 nM. In embodiments, the antibody is administered at an amount from 0.05 nM to 10 nM. In embodiments, the antibody is administered at an amount from 0.1 nM to 10 nM. In embodiments, the antibody is administered at an amount from 0.5 nM to 10 nM. In embodiments, the antibody is administered at an amount from 1 nM to 10 nM. In embodiments, the antibody is administered at an amount from 2 nM to 10 nM. In embodiments, the antibody is administered at an amount from 4 nM to 10 nM. In embodiments, the antibody is administered at an amount from 6 nM to 10 nM. In embodiments, the antibody is administered at an amount from 4 nM to 10 nM. In embodiments, the antibody is administered at an amount from 8 nM to 10 nM. In embodiments, the antibody is administered at an amount of 0.01 nM. 0.05 nM. 0.1 nM. 0.5 nM, 1 nM. 2 nM. 2 nM, 4 nM, 5 nM, 6 nM, 7 nM, 8 nM, 9 nM or 10 nM.

In embodiments, the antibody is administered at an amount from about 0.01 nM to about 8 nM. In embodiments, the antibody is administered at an amount from about 0.05 nM to about 8 nM. In embodiments, the antibody is administered at an amount from about 0.1 nM to about 8 nM. In embodiments, the antibody is administered at an amount from about 0.5 nM to about 8 nM. In embodiments, the antibody is administered at an amount from about 1 nM to about 8 nM. In embodiments, the antibody is administered at an amount from about 2 nM to about 8 nM. In embodiments, the antibody is administered at an amount from about 4 nM to about 8 nM. In embodiments, the antibody is administered at an amount from about 6 nM to about 8 nM. In embodiments, the antibody is administered at an amount from about 4 nM to about 8 nM.

In embodiments, the antibody is administered at an amount from 0.01 nM to 8 nM. In embodiments, the antibody is administered at an amount from 0.05 nM to 8 nM. In embodiments, the antibody is administered at an amount from 0.1 nM to 8 nM. In embodiments, the antibody is administered at an amount from 0.5 nM to 8 nM. In embodiments, the antibody is administered at an amount from 1 nM to 8 nM. In embodiments, the antibody is administered at an amount from 2 nM to 8 nM. In embodiments, the antibody is administered at an amount from 4 nM to 8 nM. In embodiments, the antibody is administered at an amount from 6 nM to 8 nM. In embodiments, the antibody is administered at an amount from 4 nM to 8 nM.

In embodiments, the antibody is administered at an amount from about 0.01 nM to about 6 nM. In embodiments, the antibody is administered at an amount from about 0.05 nM to about 6 nM. In embodiments, the antibody is administered at an amount from about 0.1 nM to about 6 nM. In embodiments, the antibody is administered at an amount from about 0.5 nM to about 8 nM. In embodiments, the antibody is administered at an amount from about 1 nM to about 6 nM. In embodiments, the antibody is administered at an amount from about 2 nM to about 6 nM. In embodiments, the antibody is administered at an amount from about 4 nM to about 6 nM.

In embodiments, the antibody is administered at an amount from 0.01 nM to 6 nM. In embodiments, the antibody is administered at an amount from 0.05 nM to 6 nM. In embodiments, the antibody is administered at an amount from 0.1 nM to 6 nM. In embodiments, the antibody is administered at an amount from 0.5 nM to 6 nM. In embodiments, the antibody is administered at an amount from 1 nM to 6 nM. In embodiments, the antibody is administered at an amount from 2 nM to 6 nM. In embodiments, the antibody is administered at an amount from 4 nM to 6 nM.

In embodiments, the antibody is administered at an amount from about 0.01 nM to about 4 nM. In embodiments, the antibody is administered at an amount from about 0.05 nM to about 4 nM. In embodiments, the antibody is administered at an amount from about 0.1 nM to about 4 nM. In embodiments, the antibody is administered at an amount from about 0.5 nM to about 4 nM. In embodiments, the antibody is administered at an amount from about 1 nM to about 4 nM. In embodiments, the antibody is administered at an amount from about 2 nM to about 4 nM.

In embodiments, the antibody is administered at an amount from 0.01 nM to 4 nM. In embodiments, the antibody is administered at an amount from 0.05 nM to 4 nM. In embodiments, the antibody is administered at an amount from 0.1 nM to 4 nM. In embodiments, the antibody is administered at an amount from 0.5 nM to 4 nM. In embodiments, the antibody is administered at an amount from 1 nM to 4 nM. In embodiments, the antibody is administered at an amount from 2 nM to 4 nM.

In embodiments, the antibody is administered at an amount from about 0.01 nM to about 2 nM. In embodiments, the antibody is administered at an amount from about 0.05 nM to about 2 nM. In embodiments, the antibody is administered at an amount from about 0.1 nM to about 2 nM. In embodiments, the antibody is administered at an amount from about 0.5 nM to about 2 nM. In embodiments, the antibody is administered at an amount from about 1 nM to about 2 nM.

In embodiments, the antibody is administered at an amount from 0.01 nM to 2 nM. In embodiments, the antibody is administered at an amount from 0.05 nM to 2 nM. In embodiments, the antibody is administered at an amount from 0.1 nM to 2 nM. In embodiments, the antibody is administered at an amount from 0.5 nM to 2 nM. In embodiments, the antibody is administered at an amount from 1 nM to 2 nM.

In embodiments, the antibody is administered at an amount from about 0.01 nM to about 1 nM. In embodiments, the antibody is administered at an amount from about 0.05 nM to about 1 nM. In embodiments, the antibody is administered at an amount from about 0.1 nM to about 1 nM. In embodiments, the antibody is administered at an amount from about 0.5 nM to about 1 nM.

In embodiments, the antibody is administered at an amount from 0.01 nM to 1 nM. In embodiments, the antibody is administered at an amount from 0.05 nM to 1 nM. In embodiments, the antibody is administered at an amount from 0.1 nM to 1 nM. In embodiments, the antibody is administered at an amount from 0.5 nM to 1 nM.

In embodiments, the antibody is administered at an amount of about 3.15 nM. In embodiments, the antibody is administered at an amount of 3.15 nM. In embodiments, the antibody is administered at an amount of about 1.05 nM. In embodiments, the antibody is administered at an amount of 1.05 nM.

It is understood that the recombinant protein (i.e., the bispecific antibody or scFv antibody) provided herein including embodiments thereof may be administered at any of the concentrations described herein for the administration of the antibody (e.g., 0.01 nM-10 nM).

In embodiments, the antibody is administered at an amount from about 10 μg to about 500 μg. In embodiments, the antibody is administered at an amount from about 20 μg to about 500 μg. In embodiments, the antibody is administered at an amount from about 30 μg to about 500 μg. In embodiments, the antibody is administered at an amount from about 40 μg to about 500 μg. In embodiments, the antibody is administered at an amount from about 50 μg to about 500 μg. In embodiments, the antibody is administered at an amount from about 60 μg to about 500 μg. In embodiments, the antibody is administered at an amount from about 70 μg to about 500 μg. In embodiments, the antibody is administered at an amount from about 80 μg to about 500 μg. In embodiments, the antibody is administered at an amount from about 90 μg to about 500 μg. In embodiments, the antibody is administered at an amount from about 100 μg to about 500 μg.

In embodiments, the antibody is administered at an amount from about 110 μg to about 500 μg. In embodiments, the antibody is administered at an amount from about 120 μg to about 500 μg. In embodiments, the antibody is administered at an amount from about 130 μg to about 500 μg. In embodiments, the antibody is administered at an amount from about 140 μg to about 500 μg. In embodiments, the antibody is administered at an amount from about 150 μg to about 500 μg. In embodiments, the antibody is administered at an amount from about 160 μg to about 500 μg. In embodiments, the antibody is administered at an amount from about 170 μg to about 500 μg. In embodiments, the antibody is administered at an amount from about 180 μg to about 500 μg. In embodiments, the antibody is administered at an amount from about 190 μg to about 500 μg. In embodiments, the antibody is administered at an amount from about 200 μg to about 500 μg.

In embodiments, the antibody is administered at an amount from about 210 μg to about 500 μg. In embodiments, the antibody is administered at an amount from about 220 μg to about 500 μg. In embodiments, the antibody is administered at an amount from about 230 μg to about 500 μg. In embodiments, the antibody is administered at an amount from about 240 μg to about 500 μg. In embodiments, the antibody is administered at an amount from about 250 μg to about 500 μg. In embodiments, the antibody is administered at an amount from about 260 μg to about 500 μg. In embodiments, the antibody is administered at an amount from about 270 μg to about 500 μg. In embodiments, the antibody is administered at an amount from about 280 μg to about 500 μg. In embodiments, the antibody is administered at an amount from about 290 μg to about 500 μg. In embodiments, the antibody is administered at an amount from about 300 μg to about 500 μg.

In embodiments, the antibody is administered at an amount from about 310 μg to about 500 μg. In embodiments, the antibody is administered at an amount from about 320 μg to about 500 μg. In embodiments, the antibody is administered at an amount from about 330 μg to about 500 μg. In embodiments, the antibody is administered at an amount from about 340 μg to about 500 μg. In embodiments, the antibody is administered at an amount from about 350 μg to about 500 μg. In embodiments, the antibody is administered at an amount from about 360 μg to about 500 μg. In embodiments, the antibody is administered at an amount from about 370 μg to about 500 μg. In embodiments, the antibody is administered at an amount from about 380 μg to about 500 μg. In embodiments, the antibody is administered at an amount from about 390 μg to about 500 μg. In embodiments, the antibody is administered at an amount from about 400 μg to about 500 μg.

In embodiments, the antibody is administered at an amount from about 410 μg to about 500 μg. In embodiments, the antibody is administered at an amount from about 420 μg to about 500 μg. In embodiments, the antibody is administered at an amount from about 430 μg to about 500 μg. In embodiments, the antibody is administered at an amount from about 440 μg to about 500 μg. In embodiments, the antibody is administered at an amount from about 450 μg to about 500 μg. In embodiments, the antibody is administered at an amount from about 460 μg to about 500 μg. In embodiments, the antibody is administered at an amount from about 470 μg to about 500 μg. In embodiments, the antibody is administered at an amount from about 480 μg to about 500 μg. In embodiments, the antibody is administered at an amount from about 490 μg to about 500 μg.

In embodiments, the antibody is administered at an amount from about 10 μg to about 400 μg. In embodiments, the antibody is administered at an amount from about 20 μg to about 400 μg. In embodiments, the antibody is administered at an amount from about 30 μg to about 400 μg. In embodiments, the antibody is administered at an amount from about 40 μg to about 400 μg. In embodiments, the antibody is administered at an amount from about 50 μg to about 400 μg. In embodiments, the antibody is administered at an amount from about 60 μg to about 400 μg. In embodiments, the antibody is administered at an amount from about 70 μg to about 400 μg. In embodiments, the antibody is administered at an amount from about 80 μg to about 400 μg. In embodiments, the antibody is administered at an amount from about 90 μg to about 400 μg. In embodiments, the antibody is administered at an amount from about 100 μg to about 400 μg.

In embodiments, the antibody is administered at an amount from about 10 μg to about 300 μg. In embodiments, the antibody is administered at an amount from about 20 μg to about 300 μg. In embodiments, the antibody is administered at an amount from about 30 μg to about 300 μg. In embodiments, the antibody is administered at an amount from about 40 μg to about 300 μg. In embodiments, the antibody is administered at an amount from about 50 μg to about 300 μg. In embodiments, the antibody is administered at an amount from about 60 μg to about 300 μg. In embodiments, the antibody is administered at an amount from about 70 μg to about 300 μg. In embodiments, the antibody is administered at an amount from about 80 μg to about 300 μg. In embodiments, the antibody is administered at an amount from about 90 μg to about 300 μg. In embodiments, the antibody is administered at an amount from about 100 μg to about 300 μg.

In embodiments, the antibody is administered at an amount from about 10 μg to about 200 μg. In embodiments, the antibody is administered at an amount from about 20 μg to about 200 μg. In embodiments, the antibody is administered at an amount from about 30 μg to about 200 μg. In embodiments, the antibody is administered at an amount from about 40 μg to about 200 μg. In embodiments, the antibody is administered at an amount from about 50 μg to about 200 μg. In embodiments, the antibody is administered at an amount from about 60 μg to about 200 μg. In embodiments, the antibody is administered at an amount from about 70 μg to about 200 μg. In embodiments, the antibody is administered at an amount from about 80 μg to about 200 μg. In embodiments, the antibody is administered at an amount from about 90 μg to about 200 μg. In embodiments, the antibody is administered at an amount from about 100 μg to about 200 μg.

In embodiments, the antibody is administered at an amount from about 10 μg to about 120 μg. In embodiments, the antibody is administered at an amount from about 20 μg to about 120 μg. In embodiments, the antibody is administered at an amount from about 30 μg to about 120 μg. In embodiments, the antibody is administered at an amount from about 40 μg to about 120 μg. In embodiments, the antibody is administered at an amount from about 50 μg to about 120 μg. In embodiments, the antibody is administered at an amount from about 60 μg to about 120 μg. In embodiments, the antibody is administered at an amount from about 70 μg to about 120 μg. In embodiments, the antibody is administered at an amount from about 80 μg to about 120 μg. In embodiments, the antibody is administered at an amount from about 90 μg to about 120 μg. In embodiments, the antibody is administered at an amount from about 100 μg to about 120 μg.

In embodiments, the antibody is administered at an amount from about 10 μg to about 100 μg. In embodiments, the antibody is administered at an amount from about 20 μg to about 100 μg. In embodiments, the antibody is administered at an amount from about 30 μg to about 100 μg. In embodiments, the antibody is administered at an amount from about 40 μg to about 100 μg. In embodiments, the antibody is administered at an amount from about 50 μg to about 100 μg. In embodiments, the antibody is administered at an amount from about 60 μg to about 100 μg. In embodiments, the antibody is administered at an amount from about 70 μg to about 100 μg. In embodiments, the antibody is administered at an amount from about 80 μg to about 100 μg. In embodiments, the antibody is administered at an amount from about 90 μg to about 100 μg.

In embodiments, the antibody is administered at an amount of about 10 μg. 20 μg. 30 μg. 40 μg, 50 μg. 60 μg. 70 μg. 80 μg, 90 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 ug, 290 μg, 300 μg, 310 μg, 320 μg, 330 μg, 340 μg, 350 μg, 360 μg, 370 μg, 380 μg, 390 μg, 400 μg, 410 μg, 420 μg, 430 μg, 440 μg, 450 μg, 460 μg, 470 μg, 480 μg, 490 μg, or 500 μg.

In embodiments, the antibody is administered at an amount of 10 μg, 20 μg, 30 ug, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg, 250 μg, 260 μg, 270 μg, 280 μg, 290 ug, 300 μg, 310 μg, 320 μg, 330 μg, 340 μg, 350 μg, 360 μg, 370 μg, 380 μg, 390 μg, 400 μg, 410 μg, 420 μg, 430 μg, 440 μg, 450 μg, 460 μg, 470 μg, 480 μg, 490 μg, or 500 μg.

It is understood that the protein (e.g., the antibody or recombinant protein) provided herein including embodiments thereof may be administered at any of the concentrations described herein for the administration of the antibody (e.g., 10 μg-500 μg).

In embodiments, the recombinant protein or antibody is administered at an amount of about 60 μg-120 μg. In embodiments, the recombinant protein or antibody is administered at an amount of 60 μg-120 μg.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### NUMBERED EMBODIMENTS

Some embodiments include any of the following numbered embodiments:

1. An anti-V-domain Ig suppressor of T-Cell Activation (VISTA) antibody comprising a light chain variable domain and a heavy chain variable domain,
wherein said light chain variable domain comprises:
a CDR1, a CDR2, and a CDR3 as set forth in Table 5; and
wherein said heavy chain variable domain comprises:
a CDR1, a CDR2, and a CDR3 as set forth in Table 5.

2. The antibody of embodiment 1, wherein said light chain variable domain comprises an amino acid sequence with at least 80%, 85%, 90%, 91%, 92%, 93%, 94%, 95%, 96%, 97%, 98%, 99%, or 100% sequence identity with a light chain variable domain as set forth in Table 5.

3. The antibody of embodiment 1 or embodiment 2, wherein said heavy chain variable domain comprises an amino acid sequence with at least 80%, 85%, 90%, 91%, 92%, 93%, 94%, 95%, 96%, 97%, 98%, 99%, or 100% sequence identity with a heavy chain variable domain as set forth in Table 5.

4. The antibody of any one of embodiments 1-3, wherein said light chain variable domain comprises an amino acid sequence with at least 80%, 85%, 90%, 91%, 92%, 93%, 94%, 95%, 96%, 97%, 98%, 99%, or 100% sequence identity with the sequence selected from SEQ ID NO:7, SEQ ID NO:15, SEQ ID NO:23, SEQ ID NO:31, SEQ ID NO:39, SEQ ID NO:47, SEQ ID NO:55, SEQ ID NO:63. SEQ ID NO: 71, SEQ ID NO:79, SEQ ID NO:87, SEQ ID NO:95, SEQ ID NO: 103, SEQ ID NO:111, SEQ ID NO: 119, SEQ ID NO: 127, SEQ ID NO: 135, SEQ ID NO: 143, SEQ ID NO: 151, SEQ ID NO: 159, SEQ ID NO: 167, SEQ ID NO: 175, SEQ ID NO:183, SEQ ID NO: 191, SEQ ID NO: 199, SEQ ID NO:207, SEQ ID NO:215, SEQ ID NO:223, SEQ ID NO:231, SEQ ID NO:239, SEQ ID NO:247, SEQ ID NO: 255, SEQ ID NO:263, SEQ ID NO:271, SEQ ID NO:279, SEQ ID NO:287, SEQ ID NO:295, SEQ ID NO: 303, SEQ ID NO:311, SEQ ID NO:319, SEQ ID NO:327, SEQ ID NO:335, or SEQ ID NO:343.

5. The antibody of any one of embodiments 1-4, wherein said heavy chain variable domain comprises an amino acid sequence with at least 80%, 85%, 90%, 91%, 92%, 93%, 94%, 95%, 96%, 97%, 98%, 99%, or 100% sequence identity with the sequence selected from SEQ ID NO:8, SEQ ID NO:16, SEQ ID NO:24, SEQ ID NO:32, SEQ ID NO:40, SEQ ID NO:48, SEQ ID NO:56, SEQ ID NO:64, SEQ ID NO: 72, SEQ ID NO:80, SEQ ID NO:88, SEQ ID NO:96, SEQ ID NO:104, SEQ ID NO:112, SEQ ID NO: 120, SEQ ID NO:128, SEQ ID NO: 136, SEQ ID NO:144, SEQ ID NO: 152, SEQ ID NO: 160, SEQ ID NO: 168, SEQ ID NO:176, SEQ ID NO:184, SEQ ID NO:192, SEQ ID NO:200, SEQ ID NO:208, SEQ ID NO:216, SEQ ID NO:224, SEQ ID NO:232, SEQ ID NO:240, SEQ ID NO:248, SEQ ID NO: 256, SEQ ID NO:264, SEQ ID NO:272, SEQ ID NO:280, SEQ ID NO:288, SEQ ID NO:296, SEQ ID NO: 304, SEQ ID NO:312, SEQ ID NO:320, SEQ ID NO:328, SEQ ID NO:336, or SEQ ID NO:344.

6. The antibody of any one of embodiments 1-5, wherein said antibody is a humanized antibody.

7. The antibody of any one of embodiments 1-5, wherein said antibody is a chimeric antibody.

8. The antibody of any one of embodiments 1-7, wherein said antibody is an IgG.

9. The antibody of embodiment 8, wherein said antibody is an IgG1.

10. The antibody of any one of embodiments 1-5, wherein said antibody is a Fab′ fragment.

11. The antibody of any one of embodiments 1-5, wherein said antibody is a single chain antibody (scFv).

12. The antibody of any one of embodiments 1-5, wherein said light chain variable domain and said heavy chain variable domain form part of a scFv.

13. The antibody of any one of embodiments 1-12, wherein said antibody is capable of binding VISTA.

14. The antibody of any one of embodiments 1-13, wherein said antibody is bound to VISTA.

15. The antibody of embodiment 13 or embodiment 14, wherein said VISTA forms part of a cell.

16. The antibody of embodiment 15, wherein said cell is selected from a lymphoid cell, a myeloid cell, or a stem cell.

17. The antibody of embodiment 16, wherein said cell is lymphoid cell selected from a B cell or a T cell.

18. An anti-VISTA antibody, wherein the anti-VISTA antibody binds the same epitope as an antibody comprising: a heavy chain variable region domain comprising a CDR1, a CDR2, and a CDR3 as set forth in Table 5, and a light chain variable domain comprising a CDR1, a CDR2, and a CDR3 as set forth in Table 5.

19. The anti-VISTA antibody of embodiment 18, wherein the light chain variable domain comprises an amino acid sequence with at least 80%, 85%, 90%, 91%, 92%, 93%, 94%, 95%, 96%, 97%, 98%, 99%, or 100% sequence identity with the sequence of a light chain variable domain sequence as set forth in Table 5.

20. The anti-VISTA antibody of embodiment 18 or embodiment 19, wherein the heavy chain variable domain comprises an amino acid sequence with at least 80%, 85%, 90%, 91%, 92%, 93%, 94%, 95%, 96%, 97%, 98%, 99%, or 100% sequence identity with the sequence of a heavy chain variable domain sequence as set forth in Table 5.

21. An isolated nucleic acid encoding an antibody of any one of embodiments 1-20.

22. The isolated nucleic acid of embodiment 21, wherein the antibody comprises a light chain variable domain sequence and a heavy chain variable domain sequence,
wherein said light chain variable domain sequence encodes an amino acid sequence comprising:
a CDR1, a CDR2, and a CDR3 as set forth in Table 5; or
wherein said heavy chain variable domain sequence encodes an amino acid sequence comprising:
a CDR1, a CDR2, and a CDR3 as set forth in Table 5.

23. The isolated nucleic acid of embodiment 22, wherein said light chain variable domain sequence encodes an amino acid sequence with at least 80%, 85%, 90%, 91%, 92%, 93%, 94%, 95%, 96%, 97%, 98%, 99%, or 100% sequence identity with a light chain amino acid sequence as set forth in Table 5.

24. The isolated nucleic acid of embodiment 22 or embodiment 23, wherein said heavy chain variable domain encodes an amino acid sequence with at least 80%, 85%, 90%, 91%, 92%, 93%, 94%, 95%, 96%, 97%, 98%, 99%, or 100% sequence identity with a heavy chain amino acid sequence as set forth in Table 5.

25. A cell comprising said isolated nucleic acid of any one of embodiments 22-24.

26. A pharmaceutical composition comprising a therapeutically effective amount of an antibody of any one of embodiments 1-20 and a pharmaceutically acceptable excipient.

27. The pharmaceutical composition of embodiment 26, wherein said pharmaceutical composition is formulated in an aqueous solution at a concentration of 0.200 mg/mL to 2.00 mg/mL, 0.500 mg/mL to 1.50 mg/mL, 0.700 mg/mL to 0.875 mg/mL, or 0.875 mg/mL to 1.25 mg/mL.

28. The pharmaceutical composition of embodiment 26 or embodiment 27, wherein said pharmaceutical composition is formulated for intratumoral delivery.

29. The pharmaceutical composition of any one of embodiments 26-28, further comprising a therapeutically effective amount of an antineoplastic agent.

30. The pharmaceutical composition of embodiment 29, wherein the antineoplastic agent is a PD-1 pathway inhibitor.

31. The pharmaceutical composition of embodiment 29 or embodiment 30, wherein the antineoplastic agent is selected from pembrolizumab, nivolumab, cemiplimab, tislelizumab, atezolizumab, durvalumab, avelumab, dostarlimab, retifanlimab, or toripalimab.

32. A method of treating a subject in need thereof, said method comprising administering to a subject a therapeutically effective amount of the antibody of any one of embodiments 1-20.

33. A method of treating a subject in need thereof, said method comprising administering to a subject a therapeutically effective amount of the pharmaceutical composition of any one of embodiments 26-30.

34. The method of embodiment 32 or embodiment 33, further comprising administering a therapeutically effective amount of an antineoplastic agent.

35. The method of embodiment 34, wherein the antineoplastic agent is a PD-1 pathway inhibitor.

36. The method of embodiment 34 or embodiment 35, wherein the antineoplastic agent is selected from pembrolizumab, nivolumab, cemiplimab, tislelizumab, atezolizumab, durvalumab, avelumab, dostarlimab, retifanlimab, or toripalimab.

37. The method of any one of embodiments 34-36, wherein the effective amount of an antibody and the effective amount of said antineoplastic agent are a combined synergistic amount.

38. The method of any one of embodiments 32-37, wherein said subject has a tumor or a cancer, is at risk of developing the tumor or the cancer, or is suspected of having said tumor or said cancer.

39. The method of embodiment 38, wherein said cancer is selected from non-small cell lung cancer, breast cancer, ovarian cancer, renal cancer, colorectal cancer, pancreatic cancer, or a glioblastoma.

40. The method of any one of embodiments 32-39, wherein said antibody is administered to said subject at a concentration of 0.200 mg/mL to 2.00 mg/mL, 0.500 mg/mL to 1.50 mg/mL, 0.700 mg/mL to 0.875 mg/mL, or 0.875 mg/mL to 1.25 mg/mL.

41. Use of the antibody of any one of embodiments 1-20 in the manufacture of a medicament for the treatment of a tumor or cancer.

### EXAMPLES

### Example 1: Immunization with Protein

This example shows immunization of mouse subjects with anti-VISTA antibody compositions described herein.

Mouse subjects are subjected to a 1st immunization at 2 sites comprising subcutaneous injection of 30 micrograms (μg) of antibody (protein) with Freund's complete adjuvant, which comprises water in oil emulsion and inactivated and dried mycobacteria.

After 2 weeks, the subjects are then subjected to a 2nd immunization comprising a subcutaneous injection of 30 μg protein with Freund's complete adjuvant at 2 sites.

The following week, the subject is then subjected to a 3rd immunization comprising subcutaneous injection of 30 μg protein with Freund's complete adjuvant at 2 sites.

The following week, the subject is then subjected to a 4th immunization comprising subcutaneous injection of 30 μg protein with Freund's complete adjuvant at 2 sites.

The subject is subsequently subjected to a final booster immunization injected intraperitoneally using 50 micrograms (ug) of protein. After 72 hours the spleen is collected for analysis.

### Example 2: Immunization with DNA

This example shows immunization of mouse subjects with DNA compositions encoding anti-VISTA antibodies described herein.

Animal subjects are anesthetized using isoflurane and subjected to a first immunization with 50 μL of plasmid encoding an anti-VISTA antibody described herein, which is injected intradermally followed by electroporation.

Animal subjects are later anesthetized using isoflurane and subjected to a second immunization with 50 μL of plasmid encoding an anti-VISTA antibody described herein, which is injected intradermally followed by electroporation.

Animal subjects are later anesthetized using isoflurane and subjected to a third immunization with 50 μL of plasmid encoding an anti-VISTA antibody described herein, which is injected intradermally followed by electroporation.

Animal subjects are later anesthetized using isoflurane and subjected to a fourth immunization with 50 μL of plasmid encoding an anti-VISTA antibody described herein, which is injected intradermally followed by electroporation.

Finally, animal subjects are subjected to a final booster injections, which comprises delivering a composition comprising an anti-VISTA antibody described herein via intraperitoneal injection, Seventy-two hours later, the spleen is collected for analysis.

### Example 3: Fusion Via PEG

This example shows method steps useful in the production of antibody compositions described herein.

As described herein, a HAT medium can comprise hypoxanthine, aminopterin, and thymidine. HAT medium can be useful as a selection medium, e.g., for use in the culture of hybridomas. Polyethylene glycol (PEG) is an additive that can be useful in producing antibody compositions described herein. Formulations of media used for antibody production can include hybridoma fusion and cloning supplement (HFCS) and/or fetal bovine serum (FBS).

HAT medium supplemented with HFCS and 10% FBS (HAT10 medium with HFCS) is prepared for fusion experiments using a method comprising: providing lyophilized HAT medium, reconstituting the vial of lyophilized HAT medium with 10 mL of complete RPMI medium (Roswell Park Memorial Institute 1640 medium). This reconstituted HAT medium stock solution is treated as 50× concentrated. 100 milliliters (mL) of final culture medium is prepared by adding 10 mL of fetal bovine serum, 2 mL of reconstituted HAT medium stock solution, 2 mL of HFCS, and 76 mL of complete RPMI medium. HAT medium, PEG, FBS, and HFCS are warmed in a water bath in advance of preparation of HAT10 medium with HFCS.

Myeloma Cell Preparation. Myeloma cells are lifted from culture using a cell scraper and transferred to 50 mL conical centrifuge tubes. Cells are centrifuged at 1500 rpm for 4 minutes. Supernatant is then discarded, and pelleted cells are resuspended by flicking the centrifuge tube for 30 seconds(s). Thirty milliliters of complete RPMI is added to resuspended myeloma cells. RPMI is pipetted up and down to mix the cells, and cell count is performed. Myeloma cells are washed two more times for a total of three washes with complete RPMI to reduce or remove fetal bovine serum. In some cases, reduction or removal of FBS can aid in reducing or avoiding potentially adverse effects of FBS on the action of PEG. After the third RPMI wash, 45 mL of complete RPMI is added to the cells and the conical centrifuge tube is stored inside of a cell culture incubator until ready.

Preparation of splenocytes. Mice are selected for splenocyte harvest by subjecting blood samples to ELISA screening. The spleen is surgically removed from the selected mouse using sterile technique and placed in a glass petri dish containing 5 mL RPMI. The spleen is, macerated with a sterile glass plunger under sterile conditions. The resulting macerate is transferred to a 15 mL conical centrifuge tube, with care taken to minimize transfer of spleen pieces to the centrifuge tube. Macerate is allowed to sediment in the centrifuge tube for 5 minutes and then supernatant is collected from the centrifuge tube and transferred to a new 15 mL conical centrifuge tube. The centrifuge tube containing the supernatant is centrifuged at 1500 rpm for 4 min. If the cell pellet has a reddish appearance, the cell pellet is resuspended and washed with RPMI and then repelleted before counting. The cell pellet is resuspended in 5 mL of RPMI medium and a cell count is performed.

Prepared myeloma cells (1-3×10⁷ cells) and splenocyte cells are combined in one 50 ml conical centrifuge tube, and the cells are centrifuged at 1500 rpm for 10 min. Supernatant is then removed. Pelleted cells are gently tap to resuspend, being careful not to spread too much of the cells along tube wall.

Fusion with PEG. PEG solution is preheated to 37 degrees C. One milliliter of PEG is added dropwise to resuspended cell mixture while gently rotating tube for approximately 60 seconds. One milliliter of RPMI medium dropwise by gently rotating tube for 60 seconds. Two milliliters of RPMI medium is added dropwise by gently rotating tube for 120 seconds. Add 4 mL of RPMI medium dropwise by gently rotating tube for 4 minutes. Add up to 50 mL of RPMI and centrifuge for 5 minutes at 1500 rpm. Discard the supernatant, add fresh medium, and gently tap the tube to resuspend. At least two more times, cells are resuspended in up to 50 mL of RPMI and centrifuged for 5 minutes at 1500 rpm. After the final centrifugation, the supernatant is discarded, 100 mL of the appropriate medium (HAT10 medium with HFCS) is added to the pellet, and the tube is gently tapped to resuspend the pellet. The resuspended sample is split between two tubes and brought up to 50 mL total volume in each conical using HAT10 medium with HFCS.

Cells are then plated into ten to fifteen 96-well culture plates with lids, by transferring two drops from a transfer pipette per well and adding additional HAT10 medium with HFCS. Culture plates are placed into cell culture incubator at 37° C. to undergo fusion. Five to seven days after fusion, mapping is performed. The number of clones in each well of each 96-well plate is noted. On the day of mapping, 3-4 drops of fresh HAT10 media are added to each well by transferring culture medium from the bottle to a 50 mL conical tube and dispensing the medium to the culture wells with a disposable plastic transfer pipette. Culture medium is changed one day before screening. Ten to fifteen ELISA plates are sensitized and blocked with target antigen one day before screening. After 10 days of fusion, ELISA screening is performed with supernatant.

### Example 4: Subcloning Polyclonal Hybridomas to Produce Monoclonal Antibodies

This example shows method steps useful in producing monoclonal antibodies described herein.

Hybridomas are resuspended after screening. Cells are then counted to calculate the volume needed to dispense 100 cells. Fifteen-milliliter conical tubes are filled with 10 mL culture medium, and then 100 cells are added to each 15 ml conical tube after mixing the hybridomas well. The diluted cell suspension is then poured into a sterile reservoir. The cells are mixed well in the reservoir with a multichannel pipette and 100 microliters (ul) of the suspension is pipetted into each well of a 96 well tissue culture plate. Plates are then labeled according to clones and clones are mapped 10-15 days after plating. Fifteen to twenty days after plating, supernatant is screened for target binding. Cultures are subcloned again, focusing on high expression wells. Mapping and screening steps are then repeated.

### Example 5: ELISA for Primary and Confirmation Screening in Hybridoma Supernatants/Antibodies

This example shows methods of performing enzyme-linked immunosorbent assays (ELISA) useful in determining effects of compositions described herein on tumor cell and cancer cell proliferation.

ELISA plates are coated with 50 microliters per well (μl/well) of antigen (2 μg/ml in Absorption Buffer) and incubated for 2 hours (H) at 37° C. or overnight at 4° C. Plates are washed 3 times with 200 μl PBS-T 0.1%. Then 200 μl/well of 5% NFDM is added to the ELISA plates, and plates are incubated for 1 hour (H) at 37° C. Plates are then washed 3 times with 200 μl/well PBS-T 0.1%. Then 50 μl/well of hybridoma supernatant or purified antibodies (diluted to from 1 μg/ml-10 μg/ml) or serum (1/50-1/1000 dilution) in PBS. Plates are incubated for 1 hour (1 H) at 37° C. and then plates are washed 3 times with 200 μl PBS-T 0.1%. Then 50 μl/well of anti-mouse IgG HRP-conjugate is diluted 1:8000 in 1×PBS. Plates are incubated for 1 hour at 37° C. and washed 3 times with 200 μl PBS-T 0.1%. Then 50 μl/well TMB is added and plates are incubated for 5 minutes in dark, after which time 50 μl/well of Stop Solution is added. Plates are analyzed in plate reader at 450 nm wavelength.

### Example 6: Adhesion Assay Protocol

This example shows adhesion assays useful in determining effects of compositions described herein on tumor cell and cancer cell proliferation.

Day 0: Cells are serum starved overnight.

Day 1: Antibodies are prepared in serum-free media at double the final concentration (e.g., 10 μg/ml in an experiment wherein the desired final concentration is 5 μg/ml), to a final volume of 250 μL. Cells are suspended at 400,000 cells/mL in serum-free cell media. Then, 250 μL of the cell suspension is added to 250 μL of antibody mix, and the total volume is incubated at room temperature (RT) for 1 hour (hr). In the meantime, plates are coated with 100 μL of 40 μg/ml collagen type I diluted in PBS. Plates are incubated for 1 hr at 37° C. After one hour, 100 μL of collagen is removed from the plate using a pipette. Then, 100 uL of the cells and antibody mixture is pipetted to each well and then incubated for 40 minutes at 37° C., 5% CO₂.

If preferred, the following steps can be performed in non-sterile conditions. Cells are washed 2× with 100 μL of 1×PBS using a multichannel pipette. Then, 100 μL of crystal violet dye is added to each well and incubated for 10 minutes at RT. After incubation, 100 μL of crystal violet is removed from the wells, and the cells are washed 4× with 200 μL of de-ionized (DI) water. A cotton swab is used to remove any crystal violet from the sides of the wells after the washes, with care taken to avoid touching cells at bottom of plate. Then 200 μL of 10% acetic acid is added to each well and incubated for 10 minutes at RT on a shaker. Data is collected using a plate reader at 560 nm wavelength.

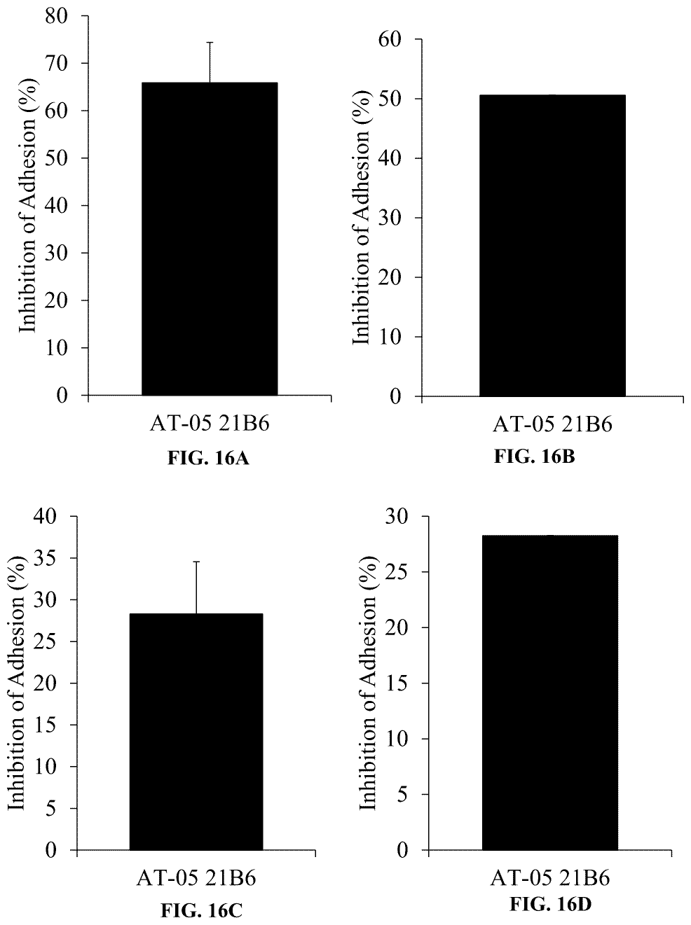

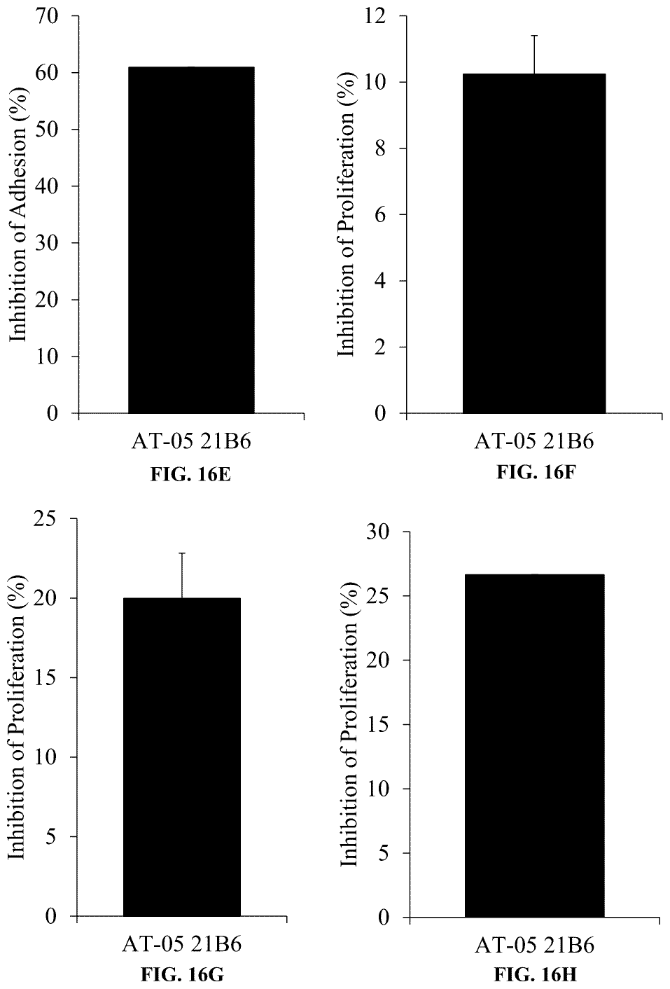

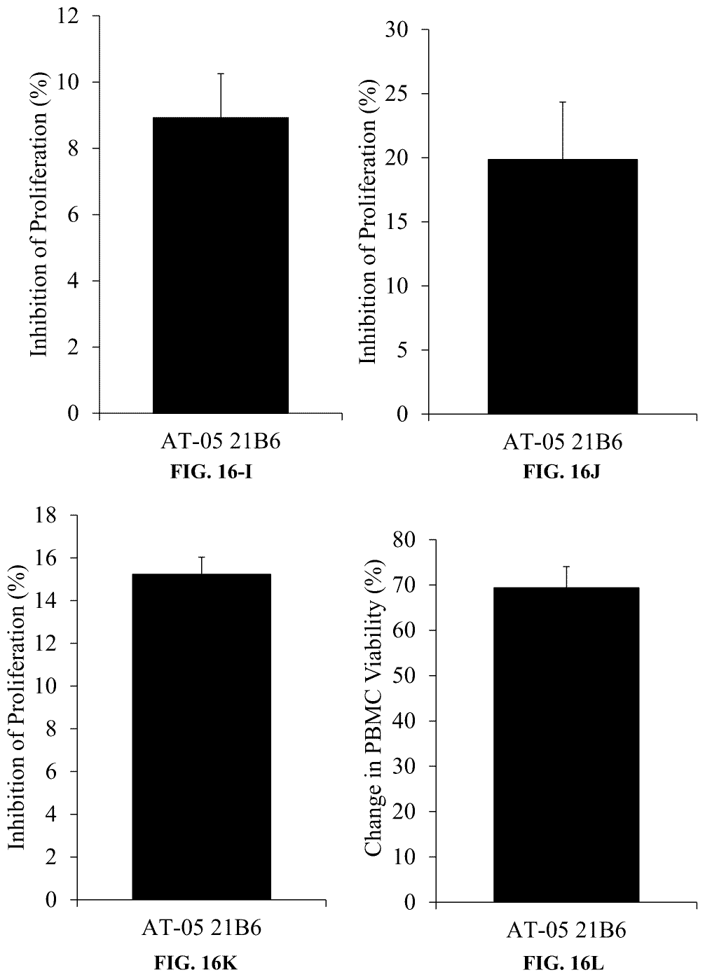

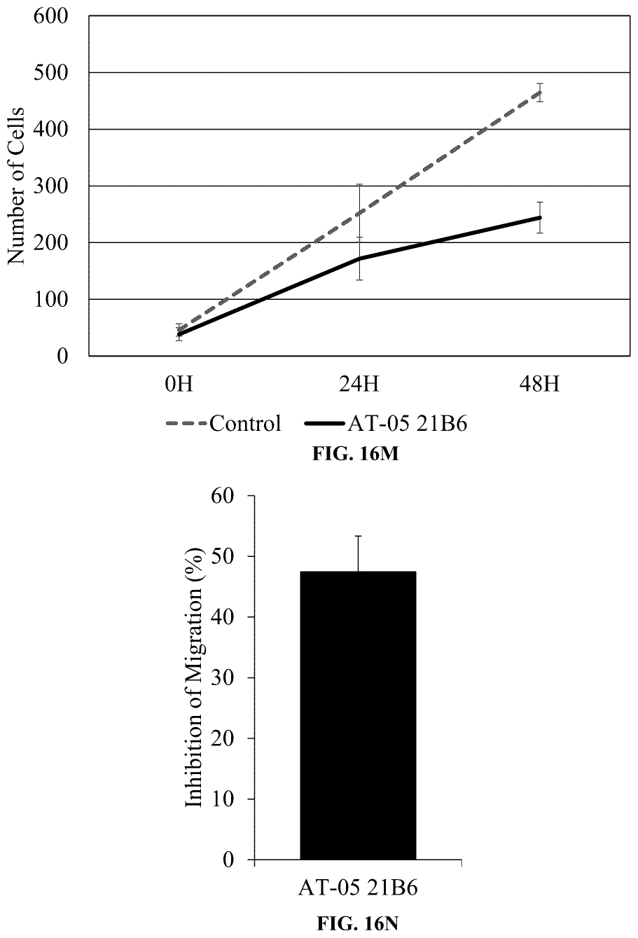

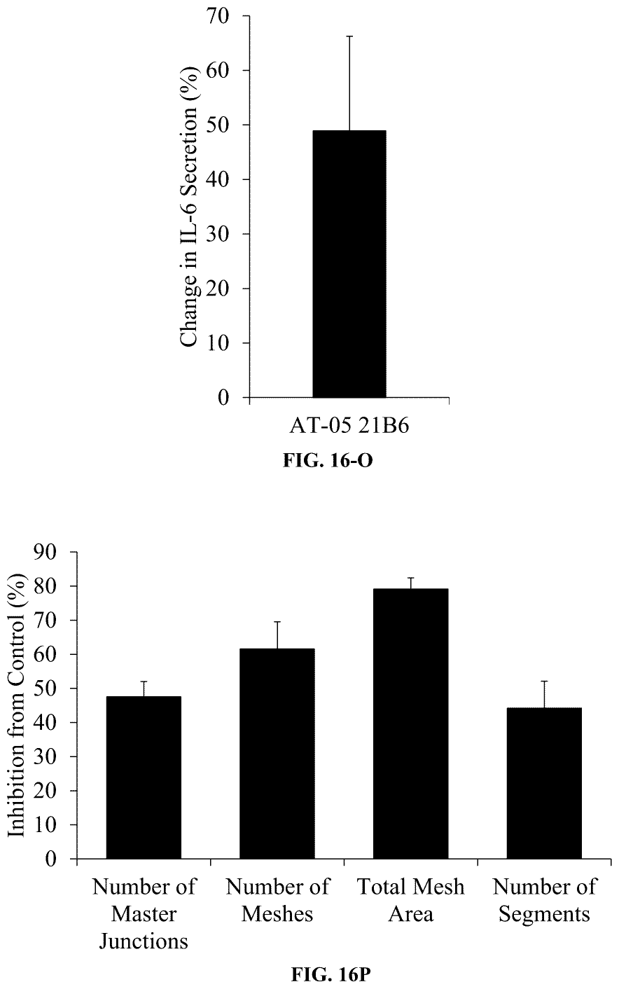

Results for AT-05.00 anti-VISTA antibodies show an inhibition of adhesion in non-small cell (NSC) lung cancer (A549) cells (21B6 clone, FIG. 16A), breast cancer (MCF-7) cells (21B6 clone, FIG. 16B), ovarian cancer (Ovcar-3) cells (21B6 clone, FIG. 16C), colorectal cancer (SW480) cells (21B6 clone, FIG. 16D), and glioblastoma cancer (U87) cells (21B6 clone, FIG. 16E), relative to control treatment.

### Example 7: Proliferation Assay

This example shows proliferation assays useful in determining effects of compositions described herein on tumor cell and cancer cell proliferation.

Day 0: Cells are serum starved overnight in a flask.

Day 1: Antibody/antigen samples are prepared in microcentrifuge tubes using 0.5% FBS, phenol red-free media such that the antibody samples are prepared at a final volume of 250 uL. Cells are resuspended at a final density of 400,000 cells/ml, and 250 μL of the cell suspension is added to 250 μL of the antibody/antigen mix. Then, 100 μL of the mixture of cells and antibody samples are added per well. A “cells-only” control and a “media-only” control are included as negative control groups, and the cultures are incubated at 37° C. and 5% CO₂ for 24 hours.

Day 2: After the 24 hour incubation, 10 uL 3-(4,5-dimethylthiazol-2-yl)-2,5-diphenyl-2H-tetrazolium bromide (MTT) is added to each well and then incubated for 2 hours at 37° C. and 5% CO₂ in the dark (e.g., by protecting the wells from light using foil or other opaque covering). After 2 hours, the supernatant media is removed from the wells, and 100 μL DMSO is added to the wells. The plate is placed on a shaker for 10 minutes, and light absorbance is determined at 560 nm using a plate reader.

### Calculations

$$\begin{matrix}\% cytotoxicity = 100 \times ( control - sample ) \\ Proliferation = 100 * ( ( sample - control ) / control )\end{matrix}$$

Resulting data were normalized by first subtracting measured background media absorbance (“media only” control sample) from sample and control values.

MTT Solution Preparation. For MTT proliferation assays, lyophilized MTT powder was resuspended in a flask or 50 mL conical centrifuge tube with sterile 1×PBS to a final concentration of 5 mg/ml under sterile conditions. The resuspended MTT is wrapped in foil to protect it from light. Vortex to resuspend up to several minutes.

Results for AT-05.00 anti-VISTA antibodies show an inhibition of proliferation in non-small cell (NSC) lung cancer (A549) cells (21B6 clone, FIG. 16F), pancreatic cancer (Capan-2) cells (21B6 clone, FIG. 16G), ovarian cancer (Ovcar-3) cells (21B6 clone, FIG. 16H), renal cancer (SKRC-18) cells (21B6 clone, FIG. 16-I), colorectal cancer (SW480) cells (21B6 clone, FIG. 16J), and glioblastoma cancer (U87) cells (21B6 clone, FIG. 16K), relative to control treatment.

### Example 8: Peripheral Blood Mononuclear Blood Cell (PBMC) Viability Assay

Viability of peripheral mononuclear blood cells (PMBC) was assessed after exposure to antibody compositions described herein or control treatment. 5×10⁴ PBMCs (Zen-Bio) were plated into a 96 well plate in RPMI-1640 supplemented with 10% FBS. Monoclonal antibodies were added to wells in triplicate to the indicated concentration. After 5 days of culture, viability was assessed by adding 20 uL CellTiter 96® AQueous One Solution (Promega) MTS reagent to 100 uL culture volume. After 2 hours, MTS signal was read at 490 nM using a microplate Reader.

Peripheral mononuclear blood cells showed increased viability when treated with AT-05.00 anti-VISTA antibodies (which are also called 21B6 clone antibodies and which are also referenced herein as “AT-05 21B6”) described herein (FIG. 16L), relative to control treatment.

### Example 9: Wound Healing Migration Assay

This example shows in vitro cell migration assays useful in determining effects of compositions described herein on tumor cell and cancer cell proliferation.

Tumor or cancer cells are resuspended at a final density of 400,000 cells/ml in culture media with 10% FBS before cells are seeded at a density of 40,000 cells/well in Platypus Oris Pro Cell Migration plates by adding 50 μL of the cell suspension on both sides of the stopper in each well. Cultures are incubated at 37° C. and 5% CO₂ for 2 hours. After the 2 hour incubation, 100 μL of the monoclonal antibody (mAb) is added to the plate (50 μL on each side of the stopper). After adding the antibody mix, the culture is incubated for 30 minutes with the stoppers still attached to the wells. After the 30 minute incubation, the stoppers are removed from the wells (0 hour (“0H”) timepoint), and the plates are imaged. Wells are imaged again at 24, 48, and 72 hour time points (“24H”, “48H”, and “72H”, respectively). An empty well is also imaged at each time point to measure the exclusion area of the well for use in calculating cell migration. Images are analyzed using ImageJ to determine cell migration.

### Example 10: Invasion Assay

This example shows in vitro cell invasion assays useful in determining effects of compositions described herein on tumor cell and cancer cell migration. Results for AT-05.00 anti-VISTA antibodies show an inhibition of invasive migration in renal cancer (SKRC-18) cells (21B6 clone, FIG. 16M and FIG. 16N) relative to control treatment.

### Example 11: IL-6 Cytokine Level Measurement

This example shows in vitro cytokine production assays useful in determining effects of compositions described herein on tumor cell and cancer cell proliferation.

Cytokine level measurements using an immunoassay (ELISA) were carried out following the manufacturer's instructions (R&D Systems). Briefly, wells of a high-binding 96-well immunoassay plate were coated with an ELISA coating (capture) antibody for one hour at 37° C. followed by three PBS washes. Wells were blocked with 1% BSA for one hour at 37° C. followed by three PBS washes. Conditioned media from cells treated with monoclonal antibody were incubated in the coated/blocked wells of the immunoassay plate for one hour at 37° C. Wells were again washed three times with PBS followed by the addition of biotinylated detection antibody. After a one hour incubation at 37° C., the wells were washed three times with PBS. Streptavidin conjugated horse radish peroxidase (HRP) was added to the wells and after a 30 minute room temperature incubation the wells were washed three times with PBS. The immunoassay was developed with the addition of 3,3′,5,5′-Tetramethylbenzidine (TMB) substrate and incubated at room temperature for 15-30 minutes before stopping the reaction and reading the plate on a microplate reader at a wavelength of 450 nm.

Results for AT-05.00 anti-VISTA antibodies show an inhibition of IL-6 secretion by ovarian cancer (ovcar-3) cells (21B6 clone, FIG. 16-O) relative to control treatment.

### Example 12: STAT-3 Activation Assay

This example shows in vitro assays for assessing STAT-3 signaling pathway activation, useful in determining effects of compositions described herein on tumor cell and cancer cell proliferation.

STAT3 Activation Assay (InstantOne ELISA, Invitrogen) was carried out following the manufacturer's instructions. Briefly, cells seeded in wells of a 96-well plate were subjected to different treatments of monoclonal antibodies for 20 minutes at 37° C. Cells were immediately washed with PBS and lysed by the addition of one hundred microliters of 1× lysis buffer and the plate incubated for 10 minutes at room temperature on a plate shaker. Fifty microliters of cell lysate were added to individual wells of a pre-coated microtiter plate (strip-wells, provided by the manufacturer) followed by 50 microliters of a prepared antibody cocktail and the plate was incubated at room temperature for one hour on a plate shaker. Wells were washed three times with PBS followed the addition of TMP substrate and incubated at room temperature for 15-30 minutes before stopping the reaction and reading the plate on a microplate reader at a wavelength of 450 nm.

### Example 13: Permeability Assay

This example shows in vitro assays for assessing permeability of tumor cells or cancer cells with or without treatment with antibody constructs described herein.

Tumor or cancer cells were seeded on the apical side of a Transwell polyester membrane insert with 0.4 μm pores (Millicell) in 24 wells plates at 60,000 cells/well and allowed to grow until a monolayer was formed. Dulbecco's Modified Eagle Medium (DMEM) media containing 1 mg/ml 10 kilo-Daltons (kDa) FITC-Dextran (ThermoFisher) and indicated treatment was added to the apical side of the insert and 500 μl fresh DMEM was added to the basal side. Following a 2 hour incubation at 37° C., 100 μl of media was sampled from the lower chamber (basolateral side) and FITC-Dextran was measured in arbitrary fluorescence units on a microplate reader with excitation and emission set to 490 nm and 525 nm respectively. Data was normalized to blank sample and then reported as a ratio of control treatment to antibody treatment.

### Example 14: Angiogenesis Assay

This example shows an vitro angiogenesis assay useful in determining effects of compositions described herein on angiogenic behavior of human endothelial cells in culture, including in vitro vessel segment, node, and junction formation.

First, 10 μL per well of chilled basement membrane matrix (BME, Corning) was added to a 96 well angiogenesis plate (Ibidi) on ice. Then, BME coated plates were incubated at 37° C. for at least 1 hour. Human umbilical vein endothelial cells (HUVECs) to 280,000 cells/ml in Endothelial Cell Growth Medium MV 2 (ECGMV2). Antibody constructs were prepared in ECGMV2 at double the desired final concentration (e.g., 10 μg/ml, if the desired final concentration is 5 μg/ml). Then, 200 μL of the HUVEC suspension was added to 200 μL of the antibody mix and 70 μL of the combined cells and antibody mix were added per well, before incubating overnight at 37° C. and 5% CO₂. Cell culture plates were imaged using a microscope camera at 4× magnification. Images were analyzed with ImageJ angiogenesis analyzer extension.

Results for AT-05.00 anti-VISTA antibodies (21B6 clone) showed an inhibition of angiogenic behavior exhibited by HUVECs (FIG. 16P), reducing number of nodes, number of junctions, number of master segments, and total number of segments formed by HUVECs in culture, relative to control treatment.

### Example 15: In Vivo Tumor Assay

This example shows an in vivo assay useful in determining effects of compositions described herein on tumor cell and cancer cell proliferation.

Nu/J Nude mice are injected with 5 million tumor cells subcutaneously in the flank and the cells are given time to grow into a tumor. Once the animals are ready to begin treatment, they are weighed, and tumors are measured prior to 100 μL intra tumor injections of either monoclonal antibodies at 1 mg/mL or PBS. Animals are weighed, tumor sizes are measured, and animals are injected with compositions described herein or control treatment biweekly until the tumor measures more than 20 mm in any direction, at which point the animal is sacrificed and the tumor collected for further study.

### Example 16: Flow Cytometry Cell Surface Affinity Binding

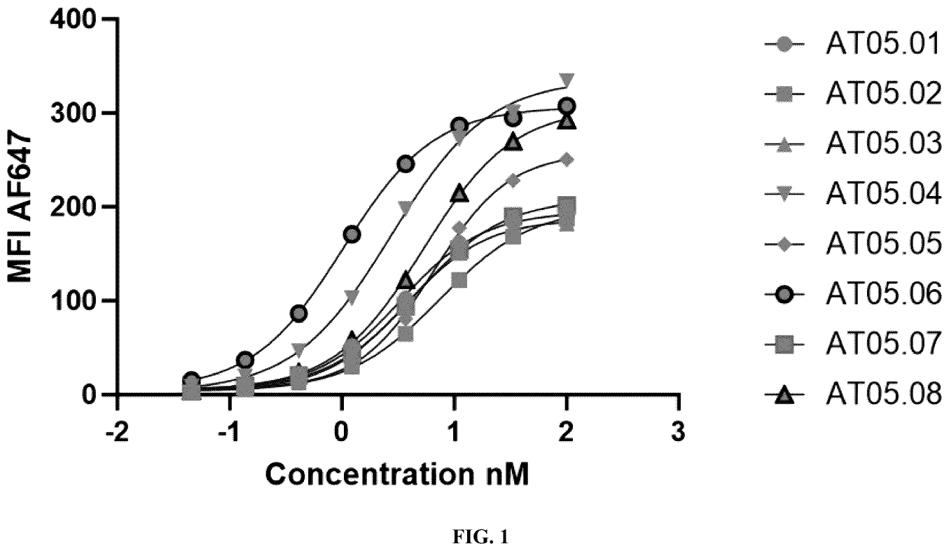

FIG. 1 shows in vitro AT05 series asset binding to cell surface VISTA expressed on THP-1 human monocytes cells. Data are presented as mean fluorescence intensity (MFI) of AlexaFluor 647 fluorophore detected during flow cytometry analysis after incubation of cells with eight AT05 series antibodies at varying concentrations of antibody (shown in nanomolar (nM) concentration). As shown in Table 1, these antibodies have EC50 affinities below 10 nanomolar in cell surface binding experiments, as measured by mean fluorescence intensity.

TABLE 1

| | Mean Fluorescence Intensity |
|---|---|
| Antibody Construct | EC50 (nM) |
| AT05.01 | 3.333 |
| AT05.02 | 7.430 |
| AT05.03 | 3.664 |
| AT05.04 | 2.749 |
| AT05.05 | 6.572 |
| AT05.06 | 1.030 |
| AT05.07 | 4.536 |
| AT05.08 | 5.301 |

### Example 17: ELISA Binding Assays to Human VISTA

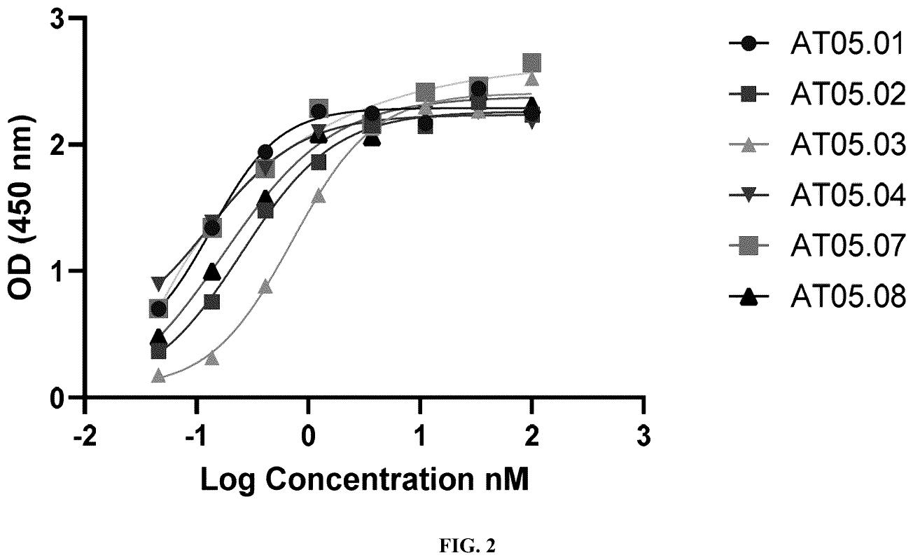

FIG. 2 shows AT05 series antibody construct binding to human VISTA protein in solid phase ELISA assays. Data is shown as a function of optical density (OD) at 450 nm wavelength for various concentrations of AT-05 series antibodies incubated at various concentrations (presented in nanomolar (nM) concentrations). As shown in Table 2, all AT05 series antibodies tested (AT05.01, AT05.02, AT05.03, AT05.04, AT05.07, and AT05.08) were found to have EC50 binding concentrations of less than 0.75 nM.

TABLE 2

| Antibody Construct | EC50 (nM) |
|---|---|
| AT05.01 | 0.1352 |
| AT05.02 | 0.2542 |
| AT05.03 | 0.7146 |
| AT05.04 | 0.1285 |
| AT05.07 | 2.66 × 10⁸ |
| AT05.08 | 0.1725 |

### Example 18: ELISA Binding to Cynomolgus VISTA

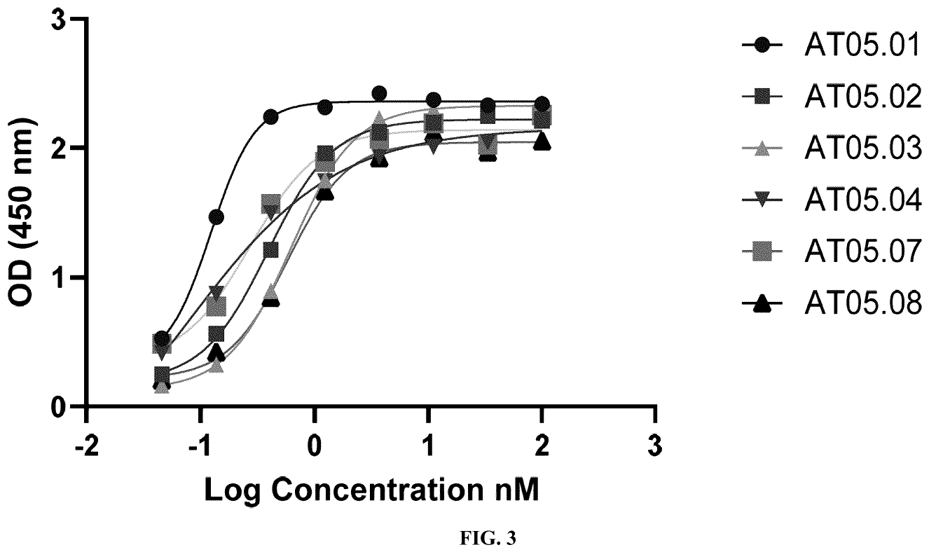

FIG. 3 shows AT05 series antibody construct binding to cynomolgus VISTA in solid phase ELISA assays. Data is shown as a function of optical density (OD) at 450 nm wavelength for various concentrations of AT-05 series antibodies incubated at various concentrations (presented in nanomolar (nM) concentrations). As shown in Table 3, all AT05 series antibodies tested (AT05.01, AT05.02, AT05.03, AT05.04, AT05.07, and AT05.08) were found to have EC50 binding concentrations of less than 0.7 nM.

TABLE 3

| Antibody Construct | EC50 (nM) |
|---|---|
| AT05.01 | 0.1235 |
| AT05.02 | 0.3913 |
| AT05.03 | 0.6201 |
| AT05.04 | 0.07628 |
| AT05.07 | 0.2736 |
| AT05.08 | 0.5788 |

### Example 19: In Vitro VISTA-VSIG3 Inhibition Assay

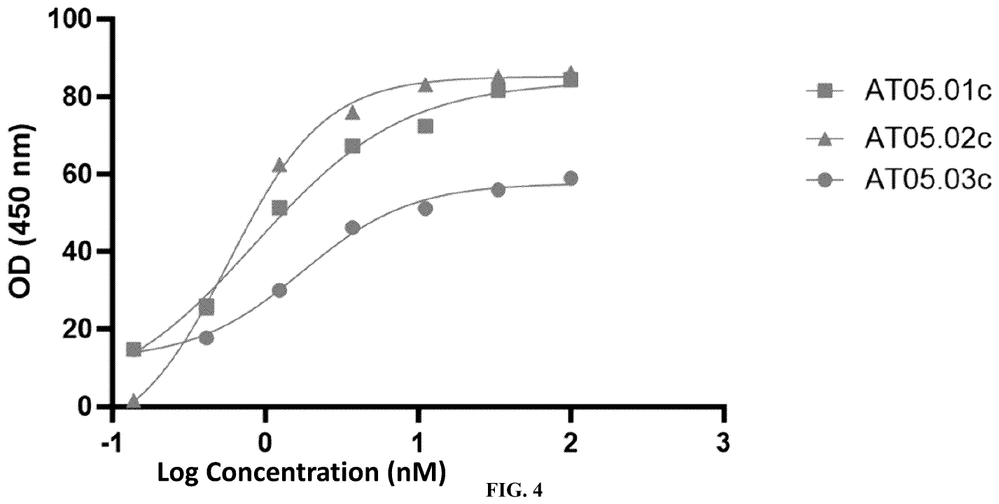

FIG. 4 shows inhibition of VISTA-VSIG3 binding was confirmed using recombinant human VISTA-Fc protein. AT05 series antibody constructs inhibit VISTA-VSIG3 binding up to 80% optical density (OD), as measured at 450 nm wavelength. Table 4 shows IC50 data for all three antibodies tested: AT05.01 (e.g., AT05.01c), AT05.02 (e.g., AT05.02c), and AT05.03 (e.g., AT05.03c). Data shows good inhibition for all tested antibody constructs. This suggests that AT05 series antibodies can be effective at blocking the inhibitory function of VISTA that leads to antitumor responses in vivo.

TABLE 4

| Antibody Construct | IC50 (nM) |
|---|---|
| AT05.03c | 1.742 |
| AT05.01c | 0.8538 |
| AT05.02c | 0.5798 |

### Example 20: TNF-Alpha ELISA Secretion Assay

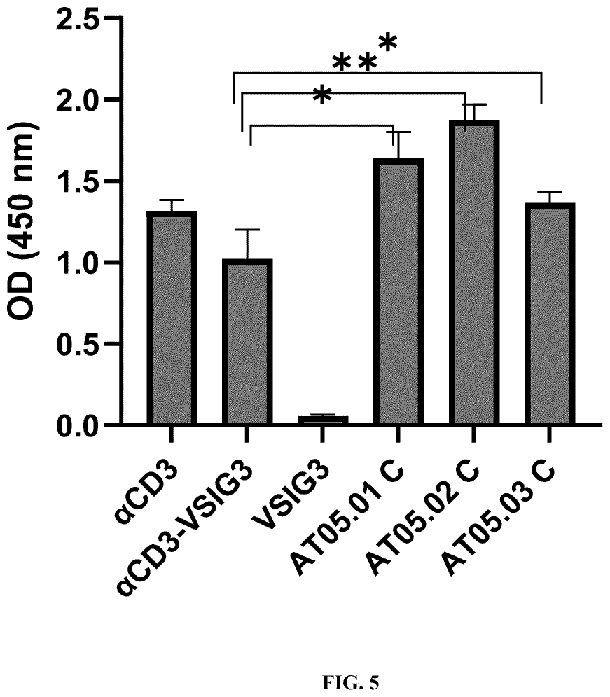

FIG. 5 shows ELISA assay data measuring TNF-alpha protein secreted by isolated peripheral blood mononuclear cells (PBMCs) cultured with AT05 series antibodies, anti-CD3 monoclonal antibody, anti-CD3 antibody and VSIG3 protein, or VSIG3 protein alone. Isolated PBMCs were cultured along with AT05 antibodies on plates coated with αCD3 monoclonal antibody and VSIG3-Fc. Anti-CD3 antibody was used as a positive control, as it binds T cell receptors, leading to TNFα secretion that can be measured by ELISA. AT05.01 (AT05.01c), AT05.02 (AT05.02c), AT05.03 (AT05.03c) chimeric antibodies all significantly increased TNFα secretion by PBMCs in this assay, when compared to the anti-CD3 antibody/VSIG3 group. Data is shown as a function of optical density (OD) at 450 nm wavelength. Asterisks indicate statistical differences in measured values.

### Example 21: Interferon Gamma Secretion ELISA Assay

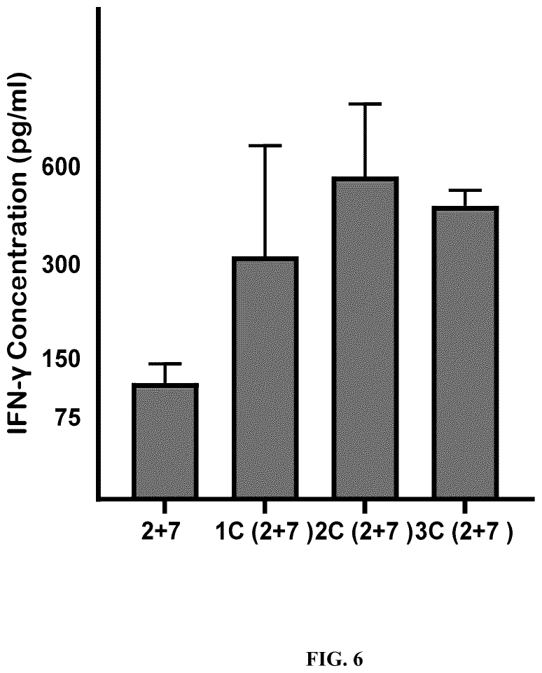

FIG. 6 shows measured interferon-gamma (IFN-γ) secretion (in picograms/milliliter: “pg/ml”) by human allogeneic mixed lymphocytic cells in ELISA assays. Anti-VISTA chimeric AT05 series antibody constructs attenuate MDSC (myeloid derived stem cells) suppression of IFN-γ release from activated T cells. Fresh PBMCs from two donors were isolated from human whole blood and mixed in a 1:1 ratio with MDSCs and AT05 antibodies were added to the mixture. In FIG. 6, “2+7” represents the presence of MDSCs and PBMCs in the assay, whereas “IC (2+7)” indicates the presence of AT05.01 antibody, “2C (2+7)” indicates the presence of AT05.02 antibody, and “3C (2+7)” indicates the presence of AT05.03 antibody. The data demonstrates that AT05 antibody constructs increase IFN-γ secretion, for example, by inhibiting MDSC-mediated suppression of interferon gamma secretion by T-cells in the PBMC population.

### Example 22: VISTA Binding and Inhibition Assays

This example shows binding of chimeric and humanized anti-VISTA antibodies to human VISTA protein in vitro.

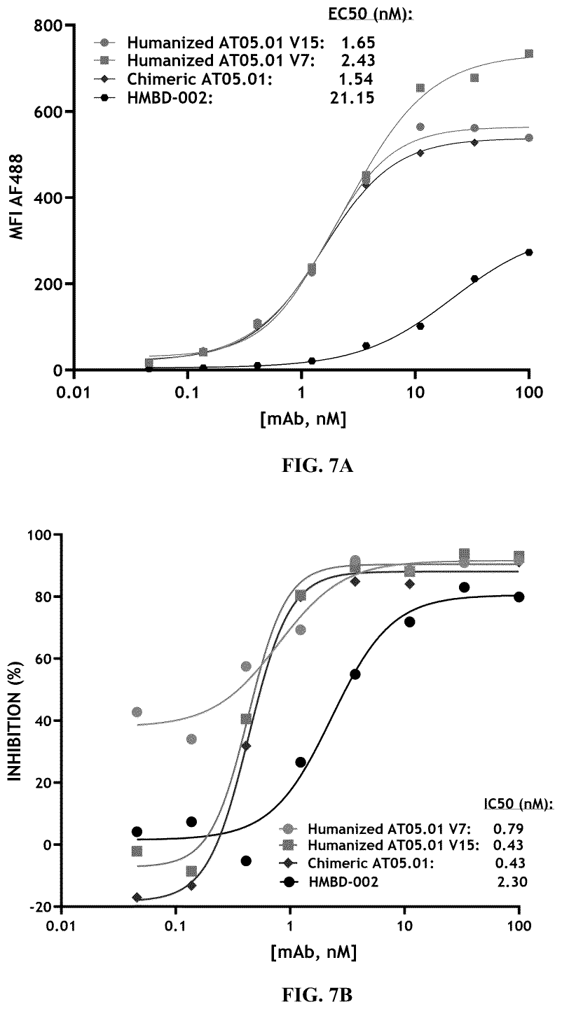

FIG. 7A shows binding of humanized anti-VISTA antibodies AT05.01v15 and AT05.01v7, chimeric anti-VISTA antibody AT05.01, and IgG4 monoclonal anti-VISTA antibody HMBD-002 (Hummingbird-002) to immobilized human VISTA protein in vitro. Each of AT05.01v15, AT05.01v7, and AT05.01 showed a greater ability to bind immobilized human VISTA protein than HMBD-002 antibody. EC50 values for each antibody were calculated from fluorescence-based binding assays. Humanized AT05.01v15 antibody was found to have an EC50 value of 1.65 nM. Humanized AT05.01v7 antibody was found to have an EC50 value of 2.43. Chimeric AT05.01 antibody was found to have an EC50 value of 1.54. HMBD-002 antibody was found to have an EC50 value of 21.15.

FIG. 7B shows inhibition of VSIG3 binding to VISTA by humanized anti-VISTA antibodies AT05.01v15 and AT05.01v7, chimeric anti-VISTA antibody AT05.01, and IgG4 monoclonal anti-VISTA antibody HMBD-002 to immobilized human VISTA protein in vitro. Each of AT05.01v15, AT05.01v7, and AT05.01 showed a greater ability to abrogate interaction of human VISTA protein with human VSIG3 protein than HMBD-002 antibody. EC50 values for each antibody were calculated from fluorescence-based binding assays. Humanized AT05.01v15 antibody was found to have an EC50 value of 0.43 nM. Humanized AT05.01v7 antibody was found to have an EC50 value of 0.79. Chimeric AT05.01 antibody was found to have an EC50 value of 0.43. HMBD-002 antibody was found to have an EC50 value of 2.3.

### Example 23: Interferon Gamma (IFN-γ) and Tumor Necrosis Factor Alpha (TNFα) Secretion Assays

This example shows in vitro data measuring interferon gamma or TNF-alpha secretion by cells after treatment with AT-05 antibody assets.

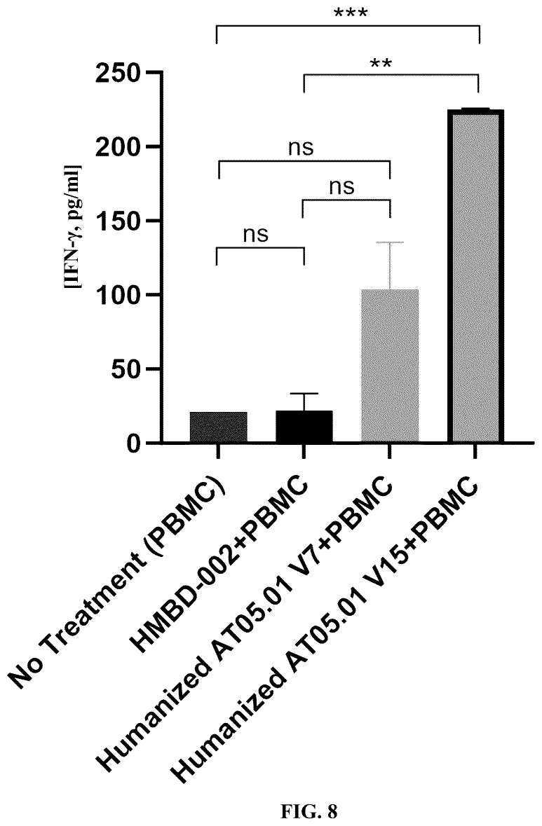

FIG. 8 shows interferon gamma secretion by peripheral blood mononuclear cells (PBMCs) after treatment with humanized anti-VISTA antibodies AT05.01v15 and AT05.01v7, or IgG4 monoclonal anti-VISTA antibody HMBD-002 (Hummingbird-002) in vitro. AT05.01v15 antibody exposure induced significantly more interferon gamma secretion from PBMCs than HMBD-002 antibody and no treatment groups. AT05.01v7 antibody exposure showed a trend of more interferon gamma secretion from PBMCs than HMBD-002 antibody and no treatment groups, but analysis did not show a significant difference. AT05 antibodies were used at a concentration of 100 nanomolar (100 nM). HMBD-002 antibodies were used at a concentration of 30 micrograms per milliliter.

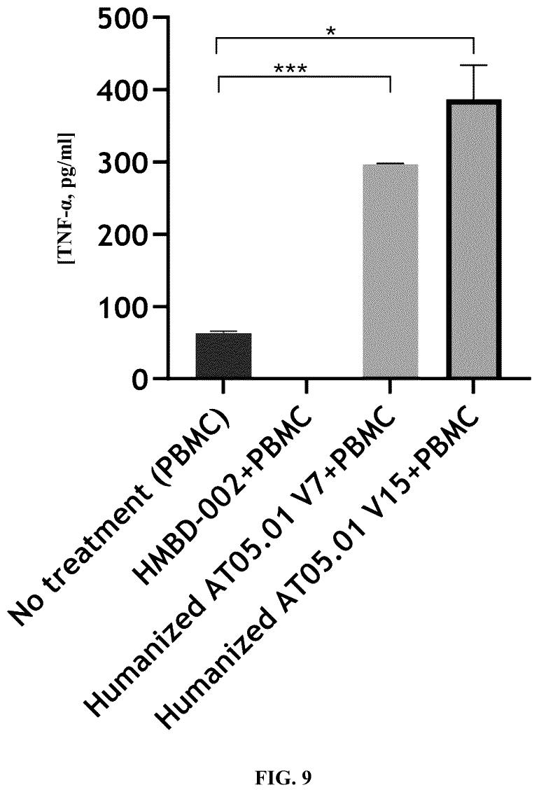

FIG. 9 shows TNF-alpha secretion by to peripheral blood mononuclear cells (PBMCs) after treatment with humanized anti-VISTA antibodies AT05.01v15 and AT05.01v7, or IgG4 monoclonal anti-VISTA antibody HMBD-002 (Hummingbird-002) in vitro. Exposure with AT05.01v15 and AT05.01v7 antibodies induced significantly more interferon gamma secretion from PBMCs than no treatment groups. AT05 antibodies were used at a concentration of 100 nanomolar (100 nM). HMBD-002 antibodies were used at a concentration of 30 micrograms per milliliter.

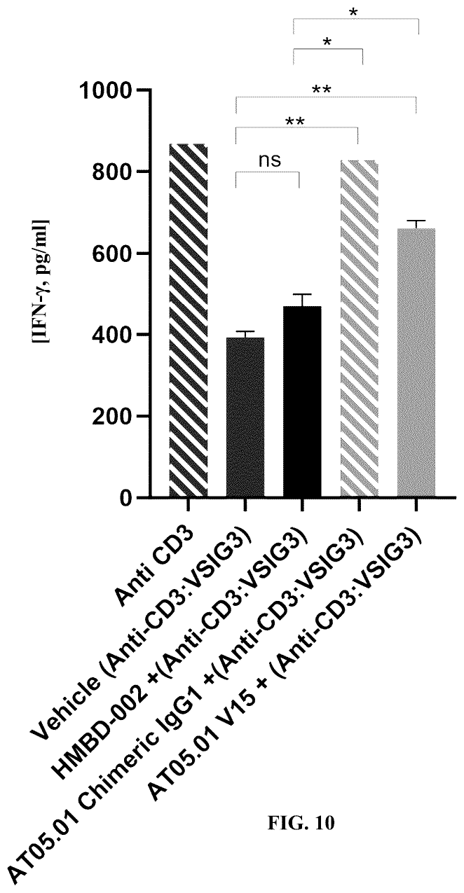

FIG. 10 shows restoration of interferon gamma secretion by T-cells after treatment with humanized anti-VISTA antibodies AT05.01v15 in vitro. AT05.01v15 antibody treatment+(Anti-CD3: VSIG3) (solid gray bar) induced significantly more interferon gamma secretion from T-cells than HMBD-002 antibody (HMBD-002+(Anti-CD3: VSIG3) and vehicle (Anti-CD3: VSIG3) control groups. AT05.01 chimeric IgG1 antibody treatment+(Anti-CD3: VSIG3) (striped gray bar) also induced significantly more interferon gamma secretion from T-cells than HMBD-002 antibody (HMBD-002+(Anti-CD3: VSIG3) and vehicle (Anti-CD3: VSIG3) control groups. AT05 antibodies were used at a concentration of 100 nanomolar (100 nM). HMBD-002 antibodies were used at a concentration of 30 micrograms per milliliter.

### Example 24: In Vivo Tumor Growth Assays

This example shows in vivo experiments evaluating tumor growth in mice after treatment with AT-05 antibody assets.

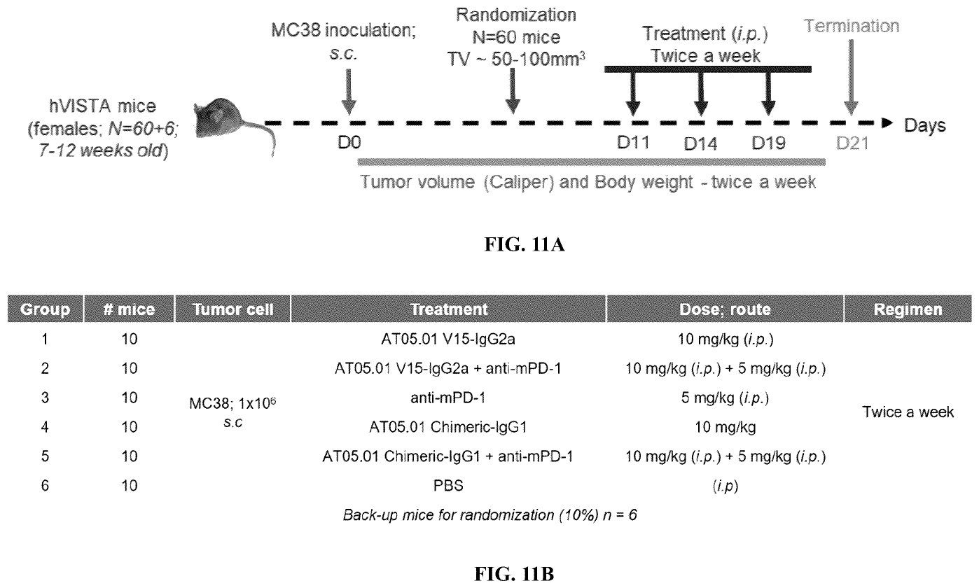

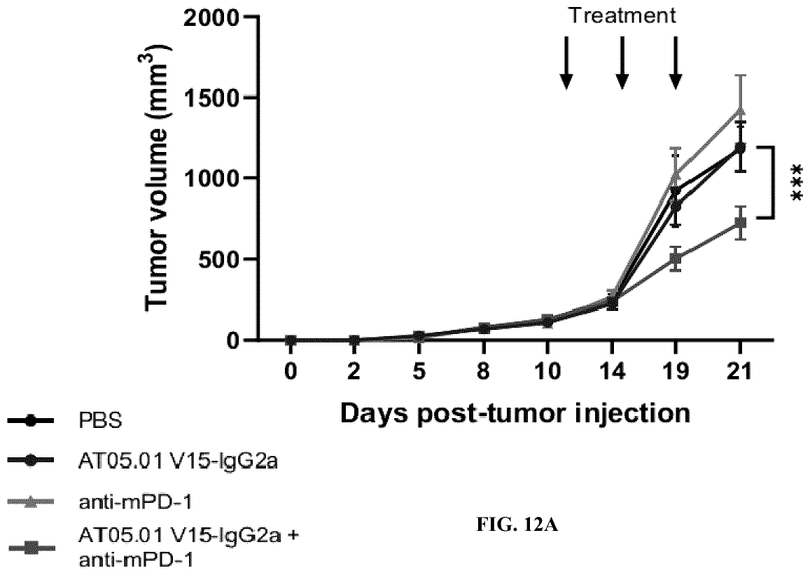

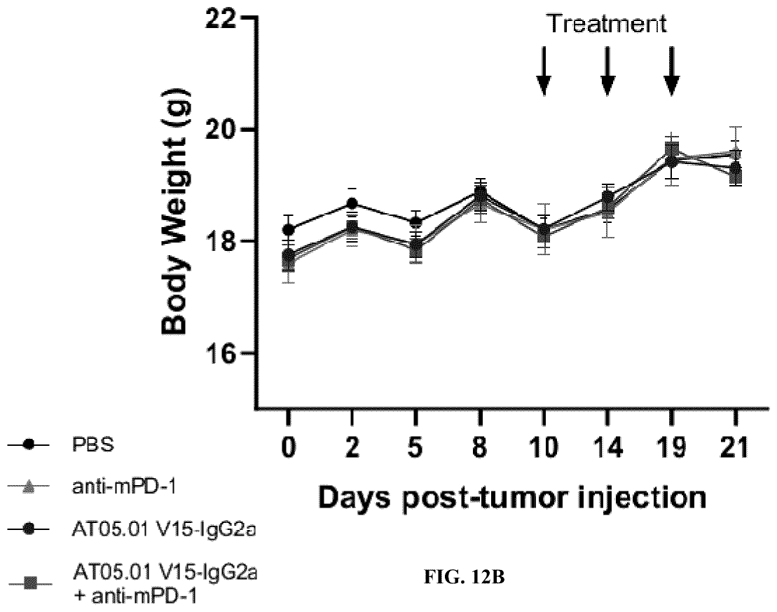

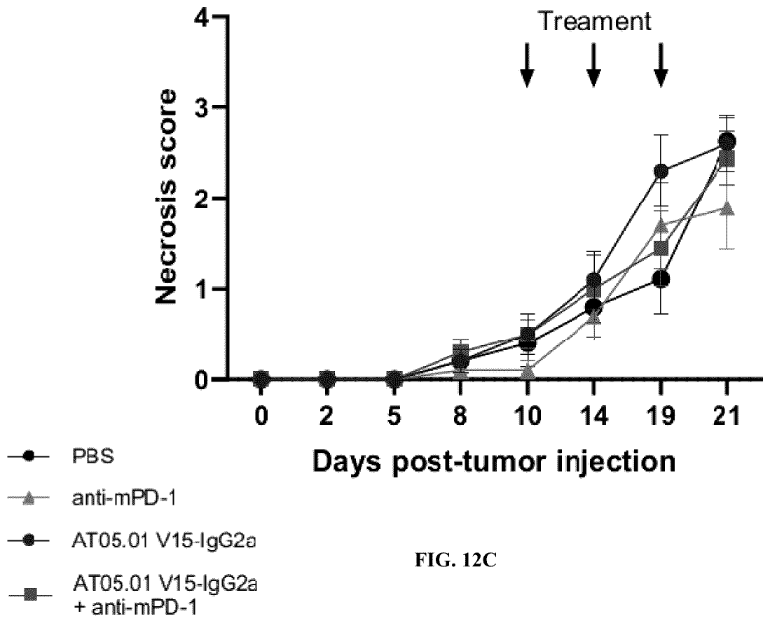

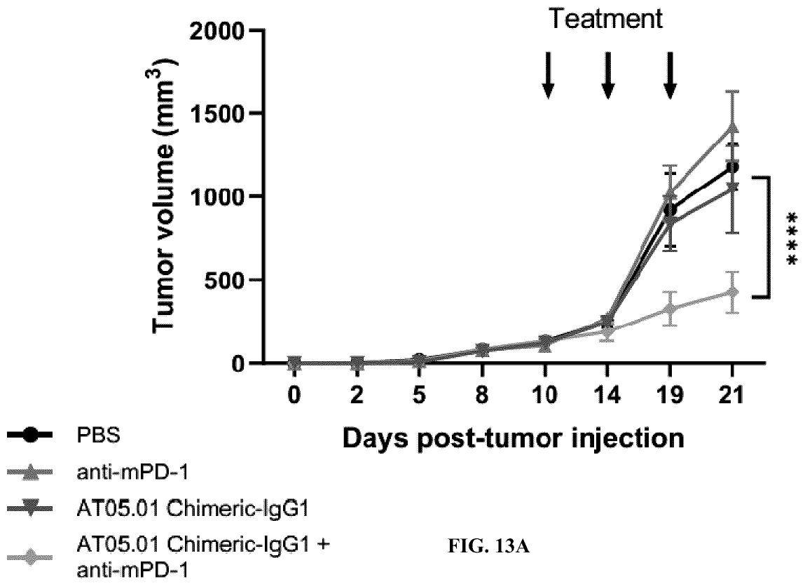

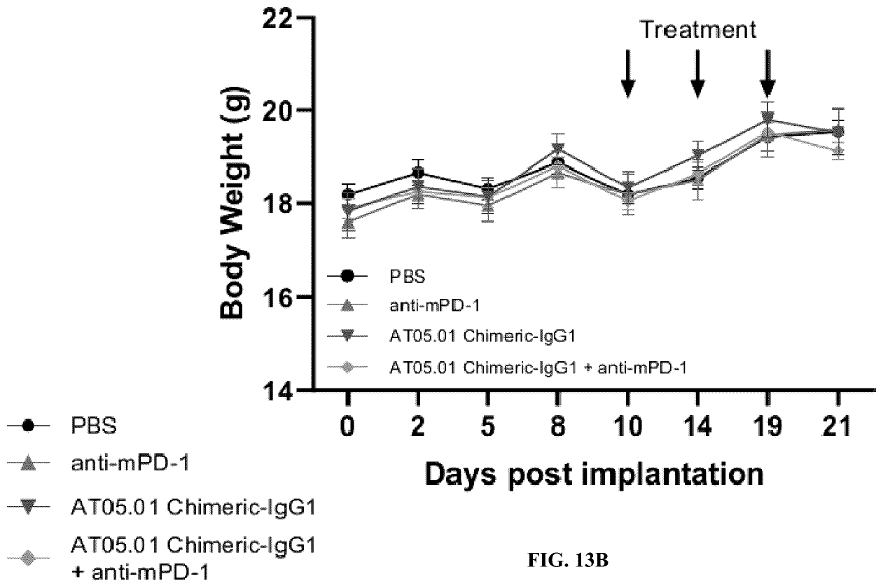

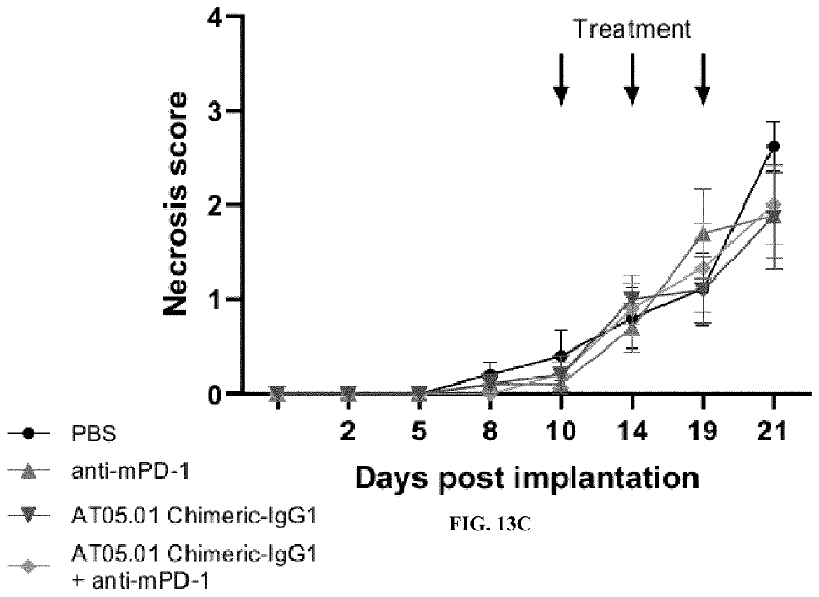

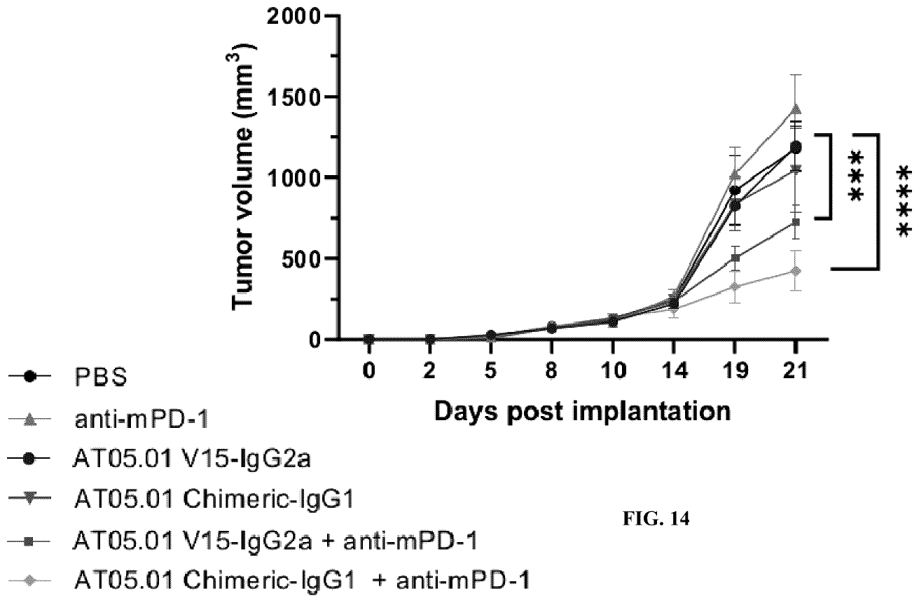

FIG. 11A an experimental design for evaluating tumor volume growth of tumors implanted in MC38 hVISTA KI mice. Mice were inoculated subcutaneously with 1 million tumor cells at Day 0 and randomized with an initial tumor volume (TV) of between 50 and 100 cubic millimeters. Intraperitoneal treatment according to groups shown in FIG. 11B were made twice a week (days 11, 14, and 19). At day 21, tumors were explanted and measured. FIG. 11B shows that each treatment group consisted of 10 mice. Groups were AT05.01V15-IgG2a antibody, AT05.01V15-IgG2a antibody plus anti-mPD-1 antibody, anti-PD-1 antibody alone, AT05.01 chimeric-IgG1 antibody, AT-05.01 chimeric-IgG1 antibody plus anti-mPD-1 antibody, and PBS vehicle treatment. FIG. 12A shows that tumor volume was significantly smaller in the AT-05.01 V15-IgG2a plus anti-mPD-1 treatment group, as compared to PBS alone treatment. FIG. 12B shows that no significant difference in mouse body weight was observed during the 21 day test period. FIG. 12C shows that no significant difference in necrosis score was measured over the 21 day test period. FIG. 13A shows that mice treated with AT05.01 chimeric-IgG1 plus anti-mPD-1 antibody had significantly smaller tumors at day 21 than PBS control-treated mice. FIG. 13B shows that there was no significant difference in mouse body weight over the 21 day test period between any two groups. FIG. 13C shows that there was no significant difference in necrosis score over the 21 day test period between groups. FIG. 14 shows that mice treated with AT05.01V15 IgG2a plus anti-mPD-1 antibody and mice treated with AT05.01 chimeric IgG1 plus anti-mPD-1 had significantly smaller tumors at day 21 than PBS control-treated mice, though the effect in mice was greater with anti-mPD-1 plus chimeric AT05.01 antibody treatments. These results suggest that AT-05 antibodies (including AT-05.01, AT-05.01v15, AT-05.01v15v1, AT-05.01v15v2, AT-05.01v15v3, AT-05.01v15v4, AT-05.01v15v5, AT-05.01v15v6, AT-05.01v15v7, AT-05.01v15v8, AT-05.01v15v9. AT-05.01v15v10, AT-05.01v15v11. AT-05.01v15v12, AT-05.01v15v13, AT-05.01v15v14, AT-05.01v15v15, AT-05.01v15v16, AT-05.01v15v17) described herein may prove effective at treating a subject (e.g., a human subject) having, at risk of having, or suspected of having a tumor or cancer (e.g., a solid tumor or cancer, such as colorectal cancer), for example, in embodiments wherein the subject is treated with the AT-05 antibody and an anti-PD-1 agent (e.g., an PD-1 pathway inhibitor, such as pembrolizumab).

### Example 25: In Vivo Tumor Growth Assays

This example shows in vivo experiments evaluating tumor growth in mice after treatment with AT-05 antibody assets.

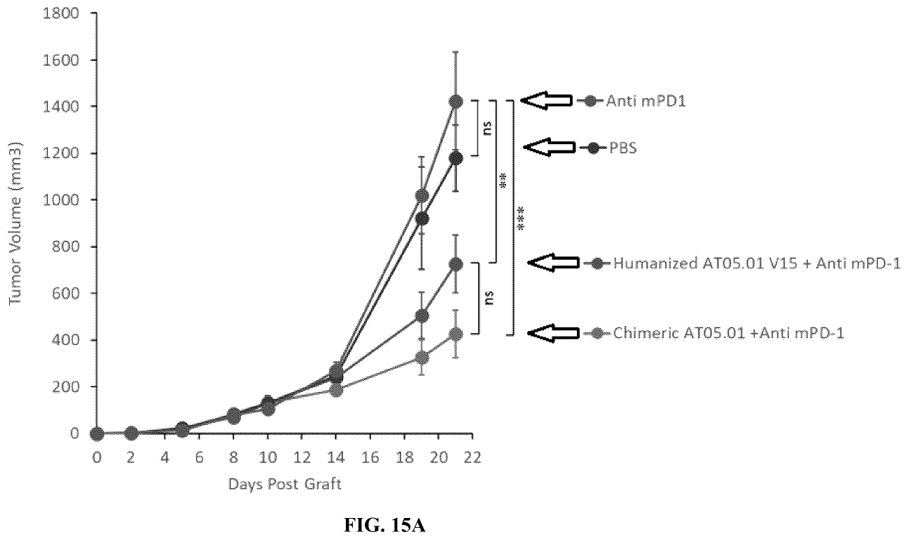

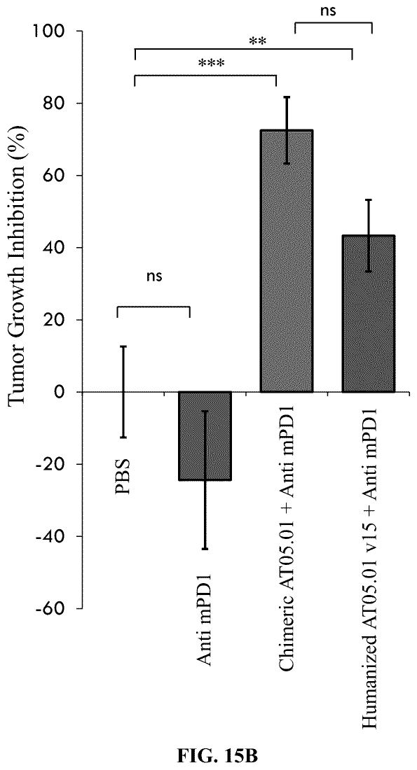

FIG. 15A shows tumor volume growth for implanted colorectal cancer tumors in MC38 hVISTA KI Mice. Treatment with anti-mPD-1 antibody yielded no significant difference in tumor size by day 21 versus PBS vehicle control treatment. Tumor size was significantly smaller in mice treated with chimeric AT05.01V15 and anti-PD-1 antibody as compared to the anti-PD-1 antibody alone. Tumor size was also significantly smaller in mice treated with humanized AT05.01V15 and anti-PD-1 antibody as compared to the anti-PD-1 antibody alone. FIG. 15B shows tumor growth inhibition normalized to PBS control treatment. Tumor growth inhibition was more than 70% higher in mice treated with chimeric AT05.01V15 and anti-PD-1 antibody as compared to the anti-PD-1 antibody alone. Tumor growth inhibition was also more than 40% higher in mice treated with humanized AT05.01V15 and anti-PD-1 antibody as compared to the anti-PD-1 antibody alone. These results suggest that AT-05 antibodies (including AT-05.01, AT-05.01v15, AT-05.01v15v1, AT-05.01v15v2, AT-05.01v15v3, AT-05.01v15v4, AT-05.01v15v5, AT-05.01v15v6, AT-05.01v15v7, AT-05.01v15v8, AT-05.01v15v9. AT-05.01v15v10. AT-05.01v15v11, AT-05.01v15v12, AT-05.01v15v13, AT-05.01v15v14, AT-05.01v15v15, AT-05.01v15v16, AT-05.01v15v17) described herein may prove effective at treating a subject (e.g., a human subject) having, at risk of having, or suspected of having a tumor or cancer (e.g., a solid tumor or cancer, such as colorectal cancer), for example, in embodiments wherein the subject is treated with the AT-05 antibody and an anti-PD-1 agent (e.g., an PD-1 pathway inhibitor, such as pembrolizumab).

TABLE 5
Sequences according to embodiments of AT-05 Assets (e.g., anti-VISTA antibodies)

| SEQ | | | | | |
|---|---|---|---|---|---|
| ID | Asset | Type | Chain | Region | Sequence |
| 1 | AT-05.01 | protein | Light | CDR1 | KASQSVDYDGDSYMN |
| 2 | AT-05.01 | protein | Light | CDR2 | GASNLES |
| 3 | AT-05.01 | protein | Light | CDR3 | QQSIEDPWT |
| 4 | AT-05.01 | protein | Heavy | CDR1 | GGYNWH |
| 5 | AT-05.01 | protein | Heavy | CDR2 | DIHKSGSTNYNPSLKS |
| 6 | AT-05.01 | protein | Heavy | CDR3 | DYYGFSWYFDV |
| 7 | AT-05.01 | protein | Light | Variable | DIVLTQSPASLAVSLGQRATISCKAS |
| | | | | | QSVDYDGDSYMNWYQQKPGQPPKL |
| | | | | | LIYGASNLESGIPARFSGSGSGRDFTL |
| | | | | | NIHPVEEEDAATYYCQQSIEDPWTFG |
| | | | | | GGTKLEIK |
| 8 | AT-05.01 | protein | Heavy | Variable | DVQLQESGPDLVKPSQSLSLTCTVTG |
| | | | | | YSITGGYNWHWIRQFPGNKLEWMG |
| | | | | | DIHKSGSTNYNPSLKSRISITRDTSKN |
| | | | | | QFFLQLNSVTTEDTATYYCARDYYG |
| | | | | | FSWYFDVWGAGTTVTVSS |
| 9 | AT-05.01v1 | protein | Light | CDR1 | KASQSVDYDGDSYMN |
| 10 | AT-05.01v1 | protein | Light | CDR2 | GASNLES |
| 11 | AT-05.01v1 | protein | Light | CDR3 | QQSIEDPWT |
| 12 | AT-05.01v1 | protein | Heavy | CDR1 | GGYNWH |
| 13 | AT-05.01v1 | protein | Heavy | CDR2 | DIHKSGSTNYNPSLKS |
| 14 | AT-05.01v1 | protein | Heavy | CDR3 | DYYGFSWYFDV |
| 15 | AT-05.01v1 | protein | Light | Variable | DIVLTQSPDSLAVSLGERATINCKAS |
| | | | | | QSVDYDGDSYMNWYQQKPGQPPKL |
| | | | | | LIYGASNLESGIPDRFSGSGSGRDFTL |
| | | | | | TISSLQAEDVATYYCQQSIEDPWTFG |
| | | | | | GGTKLEIK |
| 16 | AT-05.01v1 | protein | Heavy | Variable | QVQLQESGPGLVKPSQTLSLTCTVTG |
| | | | | | YSITGGYNWHWIRQPPGKGLEWMG |
| | | | | | DIHKSGSTNYNPSLKSRITISRDTSKN |
| | | | | | QFSLKLSSVTAADTAVYYCARDYYG |
| | | | | | FSWYFDVWGQGTTVTVSS |
| 17 | AT-05.01v2 | protein | Light | CDR1 | KASQSVDYDGDSYMN |
| 18 | AT-05.01v2 | protein | Light | CDR2 | GASNRES |
| 19 | AT-05.01v2 | protein | Light | CDR3 | QQSIEDPWT |
| 20 | AT-05.01v2 | protein | Heavy | CDR1 | GGYNWH |
| 21 | AT-05.01v2 | protein | Heavy | CDR2 | DIHKSGSTNYNPSLKS |
| 22 | AT-05.01v2 | protein | Heavy | CDR3 | DYYGFSWYFDV |
| 23 | AT-05.01v2 | protein | Light | Variable | DIVLTQSPDSLAVSLGERATINCKAS |
| | | | | | QSVDYDGDSYMNWYQQKPGQPPKL |
| | | | | | LIYGASNRESGVPDRFSGSGSGRDFT |
| | | | | | LTISSLQAEDVAVYYCQQSIEDPWTF |
| | | | | | GGGTKLEIK |
| 24 | AT-05.01v2 | protein | Heavy | Variable | QVQLQESGPGLVKPSQTLSLTCTVTG |
| | | | | | YSITGGYNWHWIRQPPGKGLEWMG |
| | | | | | DIHKSGSTNYNPSLKSRITISRDTSKN |
| | | | | | QFSLKLSSVTAADTAVYYCARDYYG |
| | | | | | FSWYFDVWGQGTTVTVSS |
| 25 | AT-05.01v3 | protein | Light | CDR1 | RASQSVDYDGDSYMN |
| 26 | AT-05.01v3 | protein | Light | CDR2 | GASNLES |
| 27 | AT-05.01v3 | protein | Light | CDR3 | QQSIEDPWT |
| 28 | AT-05.01v3 | protein | Heavy | CDR1 | GGYNWH |
| 29 | AT-05.01v3 | protein | Heavy | CDR2 | DIHKSGSTNYNPSLKS |
| 30 | AT-05.01v3 | protein | Heavy | CDR3 | DYYGFSWYFDV |
| 31 | AT-05.01v3 | protein | Light | Variable | DIVLTQSPATLSLSPGERATLSCRASQ |
| | | | | | SVDYDGDSYMNWYQQKPGQAPRLL |
| | | | | | IYGASNLESGIPARFSGSGSGRDFTLT |
| | | | | | ISSLEPEDFATYYCQQSIEDPWTFGG |
| | | | | | GTKLEIK |
| 32 | AT-05.01v3 | protein | Heavy | Variable | QVQLQESGPGLVKPSQTLSLTCTVTG |
| | | | | | YSITGGYNWHWIRQPPGKGLEWMG |
| | | | | | DIHKSGSTNYNPSLKSRITISRDTSKN |
| | | | | | QFSLKLSSVTAADTAVYYCARDYYG |
| | | | | | FSWYFDVWGQGTTVTVSS |
| 33 | AT-05.01v4 | protein | Light | CDR1 | RASQSVDYDGDSYMN |
| 34 | AT-05.01v4 | protein | Light | CDR2 | GASNRET |
| 35 | AT-05.01v4 | protein | Light | CDR3 | QQSIEDPWT |
| 36 | AT-05.01v4 | protein | Heavy | CDR1 | GGYNWH |
| 37 | AT-05.01v4 | protein | Heavy | CDR2 | DIHKSGSTNYNPSLKS |
| 38 | AT-05.01v4 | protein | Heavy | CDR3 | DYYGFSWYFDV |
| 39 | AT-05.01v4 | protein | Light | Variable | EIVLTQSPATLSLSPGERATLSCRASQ |
| | | | | | SVDYDGDSYMNWYQQKPGQAPRLL |
| | | | | | IYGASNRETGIPARFSGSGSGRDFTLT |
| | | | | | ISSLEPEDFAVYYCQQSIEDPWTFGG |
| | | | | | GTKLEIK |
| 40 | AT-05.01v4 | protein | Heavy | Variable | QVQLQESGPGLVKPSQTLSLTCTVTG |
| | | | | | YSITGGYNWHWIRQPPGKGLEWMG |
| | | | | | DIHKSGSTNYNPSLKSRITISRDTSKN |
| | | | | | QFSLKLSSVTAADTAVYYCARDYYG |
| | | | | | FSWYFDVWGQGTTVTVSS |
| 41 | AT-05.01v5 | protein | Light | CDR1 | KASQSVDYDGDSYMN |
| 42 | AT-05.01v5 | protein | Light | CDR2 | GASNLES |
| 43 | AT-05.01v5 | protein | Light | CDR3 | QQSIEDPWT |
| 44 | AT-05.01v5 | protein | Heavy | CDR1 | GGYNWH |
| 45 | AT-05.01v5 | protein | Heavy | CDR2 | DIHKSGSTNYNPSLKS |
| 46 | AT-05.01v5 | protein | Heavy | CDR3 | DYYGFSWYFDV |
| 47 | AT-05.01v5 | protein | Light | Variable | DIVLTQSPDSLAVSLGERATINCKAS |
| | | | | | QSVDYDGDSYMNWYQQKPGQPPKL |
| | | | | | LIYGASNLESGIPDRFSGSGSGRDFTL |
| | | | | | TISSLQAEDVATYYCQQSIEDPWTFG |
| | | | | | GGTKLEIK |
| 48 | AT-05.01v5 | protein | Heavy | Variable | QVQLQESGPGLVKPSQTLSLTCTVTG |
| | | | | | YSITGGYNWHWIRQPPGKGLEWIGD |
| | | | | | IHKSGSTNYNPSLKSRVTISRDTSKN |
| | | | | | QFSLKLSSVTAADTAVYYCARDYYG |
| | | | | | FSWYFDVWGQGTTVTVSS |
| 49 | AT-05.01v6 | protein | Light | CDR1 | KASQSVDYDGDSYMN |
| 50 | AT-05.01v6 | protein | Light | CDR2 | GASNRES |
| 51 | AT-05.01v6 | protein | Light | CDR3 | QQSIEDPWT |
| 52 | AT-05.01v6 | protein | Heavy | CDR1 | GGYNWH |
| 53 | AT-05.01v6 | protein | Heavy | CDR2 | DIHKSGSTNYNPSLKS |
| 54 | AT-05.01v6 | protein | Heavy | CDR3 | DYYGFSWYFDV |
| 55 | AT-05.01v6 | protein | Light | Variable | DIVLTQSPDSLAVSLGERATINCKAS |
| | | | | | QSVDYDGDSYMNWYQQKPGQPPKL |
| | | | | | LIYGASNRESGVPDRFSGSGSGRDFT |
| | | | | | LTISSLQAEDVAVYYCQQSIEDPWTF |
| | | | | | GGGTKLEIK |
| 56 | AT-05.01v6 | protein | Heavy | Variable | QVQLQESGPGLVKPSQTLSLTCTVTG |
| | | | | | YSITGGYNWHWIRQPPGKGLEWIGD |
| | | | | | IHKSGSTNYNPSLKSRVTISRDTSKN |
| | | | | | QFSLKLSSVTAADTAVYYCARDYYG |
| | | | | | FSWYFDVWGQGTTVTVSS |
| 57 | AT-05.01v7 | protein | Light | CDR1 | RASQSVDYDGDSYMN |
| 58 | AT-05.01v7 | protein | Light | CDR2 | GASNLES |
| 59 | AT-05.01v7 | protein | Light | CDR3 | QQSIEDPWT |
| 60 | AT-05.01v7 | protein | Heavy | CDR1 | GGYNWH |
| 61 | AT-05.01v7 | protein | Heavy | CDR2 | DIHKSGSTNYNPSLKS |
| 62 | AT-05.01v7 | protein | Heavy | CDR3 | DYYGFSWYFDV |
| 63 | AT-05.01v7 | protein | Light | Variable | DIVLTQSPATLSLSPGERATLSCRASQ |
| | | | | | SVDYDGDSYMNWYQQKPGQAPRLL |
| | | | | | IYGASNLESGIPARFSGSGSGRDFTLT |
| | | | | | ISSLEPEDFATYYCQQSIEDPWTFGG |
| | | | | | GTKLEIK |
| 64 | AT-05.01v7 | protein | Heavy | Variable | QVQLQESGPGLVKPSQTLSLTCTVTG |
| | | | | | YSITGGYNWHWIRQPPGKGLEWIGD |
| | | | | | IHKSGSTNYNPSLKSRVTISRDTSKN |
| | | | | | QFSLKLSSVTAADTAVYYCARDYYG |
| | | | | | FSWYFDVWGQGTTVTVSS |
| 65 | AT-05.01v8 | protein | Light | CDR1 | RASQSVDYDGDSYMN |
| 66 | AT-05.01v8 | protein | Light | CDR2 | GASNRET |
| 67 | AT-05.01v8 | protein | Light | CDR3 | QQSIEDPWT |
| 68 | AT-05.01v8 | protein | Heavy | CDR1 | GGYNWH |
| 69 | AT-05.01v8 | protein | Heavy | CDR2 | DIHKSGSTNYNPSLKS |
| 70 | AT-05.01v8 | protein | Heavy | CDR3 | DYYGFSWYFDV |
| 71 | AT-05.01v8 | protein | Light | Variable | EIVLTQSPATLSLSPGERATLSCRASQ |
| | | | | | SVDYDGDSYMNWYQQKPGQAPRLL |
| | | | | | IYGASNRETGIPARFSGSGSGRDFTLT |
| | | | | | ISSLEPEDFAVYYCQQSIEDPWTFGG |
| | | | | | GTKLEIK |
| 72 | AT-05.01v8 | protein | Heavy | Variable | QVQLQESGPGLVKPSQTLSLTCTVTG |
| | | | | | YSITGGYNWHWIRQPPGKGLEWIGD |
| | | | | | IHKSGSTNYNPSLKSRVTISRDTSKN |
| | | | | | QFSLKLSSVTAADTAVYYCARDYYG |
| | | | | | FSWYFDVWGQGTTVTVSS |
| 73 | AT-05.01v9 | protein | Light | CDR1 | KASQSVDYDGDSYMN |
| 74 | AT-05.01v9 | protein | Light | CDR2 | GASNLES |
| 75 | AT-05.01v9 | protein | Light | CDR3 | QQSIEDPWT |
| 76 | AT-05.01v9 | protein | Heavy | CDR1 | GGYNWH |
| 77 | AT-05.01v9 | protein | Heavy | CDR2 | DIHKSGSTNYNPSLKT |
| 78 | AT-05.01v9 | protein | Heavy | CDR3 | DYYGFSWYFDV |
| 79 | AT-05.01v9 | protein | Light | Variable | DIVLTQSPDSLAVSLGERATINCKAS |
| | | | | | QSVDYDGDSYMNWYQQKPGQPPKL |
| | | | | | LIYGASNLESGIPDRFSGSGSGRDFTL |
| | | | | | TISSLQAEDVATYYCQQSIEDPWTFG |
| | | | | | GGTKLEIK |
| 80 | AT-05.01v9 | protein | Heavy | Variable | QVTLKESGPALVKPTQTLTLTCTVTG |
| | | | | | YSITGGYNWHWIRQPPGKALEWMG |
| | | | | | DIHKSGSTNYNPSLKTRITISRDTSKN |
| | | | | | QFVLTMTNMDPVDTATYYCARDYY |
| | | | | | GFSWYFDVWGQGTTVTVSS |
| 81 | AT-05.01v10 | protein | Light | CDR1 | KASQSVDYDGDSYMN |
| 82 | AT-05.01v10 | protein | Light | CDR2 | GASNRES |
| 83 | AT-05.01v10 | protein | Light | CDR3 | QQSIEDPWT |
| 84 | AT-05.01v10 | protein | Heavy | CDR1 | GGYNWH |
| 85 | AT-05.01v10 | protein | Heavy | CDR2 | DIHKSGSTNYNPSLKT |
| 86 | AT-05.01v10 | protein | Heavy | CDR3 | DYYGFSWYFDV |
| 87 | AT-05.01v10 | protein | Light | Variable | DIVLTQSPDSLAVSLGERATINCKAS |
| | | | | | QSVDYDGDSYMNWYQQKPGQPPKL |
| | | | | | LIYGASNRESGVPDRFSGSGSGRDFT |
| | | | | | LTISSLQAEDVAVYYCQQSIEDPWTF |
| | | | | | GGGTKLEIK |
| 88 | AT-05.01v10 | protein | Heavy | Variable | QVTLKESGPALVKPTQTLTLTCTVTG |
| | | | | | YSITGGYNWHWIRQPPGKALEWMG |
| | | | | | DIHKSGSTNYNPSLKTRITISRDTSKN |
| | | | | | QFVLTMTNMDPVDTATYYCARDYY |
| | | | | | GFSWYFDVWGQGTTVTVSS |
| 89 | AT-05.01v11 | protein | Light | CDR1 | RASQSVDYDGDSYMN |
| 90 | AT-05.01v11 | protein | Light | CDR2 | GASNLES |
| 91 | AT-05.01v11 | protein | Light | CDR3 | QQSIEDPWT |
| 92 | AT-05.01v11 | protein | Heavy | CDR1 | GGYNWH |
| 93 | AT-05.01v11 | protein | Heavy | CDR2 | DIHKSGSTNYNPSLKT |
| 94 | AT-05.01v11 | protein | Heavy | CDR3 | DYYGFSWYFDV |
| 95 | AT-05.01v11 | protein | Light | Variable | DIVLTQSPATLSLSPGERATLSCRASQ |
| | | | | | SVDYDGDSYMNWYQQKPGQAPRLL |
| | | | | | IYGASNLESGIPARFSGSGSGRDFTLT |
| | | | | | ISSLEPEDFATYYCQQSIEDPWTFGG |
| | | | | | GTKLEIK |
| 96 | AT-05.01v11 | protein | Heavy | Variable | QVTLKESGPALVKPTQTLTLTCTVTG |
| | | | | | YSITGGYNWHWIRQPPGKALEWMG |
| | | | | | DIHKSGSTNYNPSLKTRITISRDTSKN |
| | | | | | QFVLTMTNMDPVDTATYYCARDYY |
| | | | | | GFSWYFDVWGQGTTVTVSS |
| 97 | AT-05.01v12 | protein | Light | CDR1 | RASQSVDYDGDSYMN |
| 98 | AT-05.01v12 | protein | Light | CDR2 | GASNRET |
| 99 | AT-05.01v12 | protein | Light | CDR3 | QQSIEDPWT |
| 100 | AT-05.01v12 | protein | Heavy | CDR1 | GGYNWH |
| 101 | AT-05.01v12 | protein | Heavy | CDR2 | DIHKSGSTNYNPSLKT |
| 102 | AT-05.01v12 | protein | Heavy | CDR3 | DYYGFSWYFDV |
| 103 | AT-05.01v12 | protein | Light | Variable | EIVLTQSPATLSLSPGERATLSCRASQ |
| | | | | | SVDYDGDSYMNWYQQKPGQAPRLL |
| | | | | | IYGASNRETGIPARFSGSGSGRDFTLT |
| | | | | | ISSLEPEDFAVYYCQQSIEDPWTFGG |
| | | | | | GTKLEIK |
| 104 | AT-05.01v12 | protein | Heavy | Variable | QVTLKESGPALVKPTQTLTLTCTVTG |
| | | | | | YSITGGYNWHWIRQPPGKALEWMG |
| | | | | | DIHKSGSTNYNPSLKTRITISRDTSKN |
| | | | | | QFVLTMTNMDPVDTATYYCARDYY |
| | | | | | GFSWYFDVWGQGTTVTVSS |
| 105 | AT-05.01v13 | protein | Light | CDR1 | KASQSVDYDGDSYMN |
| 106 | AT-05.01v13 | protein | Light | CDR2 | GASNLES |
| 107 | AT-05.01v13 | protein | Light | CDR3 | QQSIEDPWT |
| 108 | AT-05.01v13 | protein | Heavy | CDR1 | GGYNWH |
| 109 | AT-05.01v13 | protein | Heavy | CDR2 | DIHKSGSTNYNPSLKT |
| 110 | AT-05.01v13 | protein | Heavy | CDR3 | DYYGFSWYFDV |
| 111 | AT-05.01v13 | protein | Light | Variable | DIVLTQSPDSLAVSLGERATINCKAS |
| | | | | | QSVDYDGDSYMNWYQQKPGQPPKL |
| | | | | | LIYGASNLESGIPDRFSGSGSGRDFTL |
| | | | | | TISSLQAEDVATYYCQQSIEDPWTFG |
| | | | | | GGTKLEIK |
| 112 | AT-05.01v13 | protein | Heavy | Variable | QVTLKESGPALVKPTQTLTLTCTVTG |
| | | | | | YSITGGYNWHWIRQPPGKALEWIGD |
| | | | | | IHKSGSTNYNPSLKTRLTISRDTSKN |
| | | | | | QFVLTMTNMDPVDTATYYCARDYY |
| | | | | | GFSWYFDVWGQGTTVTVSS |
| 113 | AT-05.01v14 | protein | Light | CDR1 | KASQSVDYDGDSYMN |
| 114 | AT-05.01v14 | protein | Light | CDR2 | GASNRES |
| 115 | AT-05.01v14 | protein | Light | CDR3 | QQSIEDPWT |
| 116 | AT-05.01v14 | protein | Heavy | CDR1 | GGYNWH |
| 117 | AT-05.01v14 | protein | Heavy | CDR2 | DIHKSGSTNYNPSLKT |
| 118 | AT-05.01v14 | protein | Heavy | CDR3 | DYYGFSWYFDV |
| 119 | AT-05.01v14 | protein | Light | Variable | DIVLTQSPDSLAVSLGERATINCKAS |
| | | | | | QSVDYDGDSYMNWYQQKPGQPPKL |
| | | | | | LIYGASNRESGVPDRFSGSGSGRDFT |
| | | | | | LTISSLQAEDVAVYYCQQSIEDPWTF |
| | | | | | GGGTKLEIK |
| 120 | AT-05.01v14 | protein | Heavy | Variable | QVTLKESGPALVKPTQTLTLTCTVTG |
| | | | | | YSITGGYNWHWIRQPPGKALEWIGD |
| | | | | | IHKSGSTNYNPSLKTRLTISRDTSKN |
| | | | | | QFVLTMTNMDPVDTATYYCARDYY |
| | | | | | GFSWYFDVWGQGTTVTVSS |
| 121 | AT-05.01v15 | protein | Light | CDR1 | RASQSVDYDGDSYMN |
| 122 | AT-05.01v15 | protein | Light | CDR2 | GASNLES |
| 123 | AT-05.01v15 | protein | Light | CDR3 | QQSIEDPWT |
| 124 | AT-05.01v15 | protein | Heavy | CDR1 | GGYNWH |
| 125 | AT-05.01v15 | protein | Heavy | CDR2 | DIHKSGSTNYNPSLKT |
| 126 | AT-05.01v15 | protein | Heavy | CDR3 | DYYGFSWYFDV |
| 127 | AT-05.01v15 | protein | Light | Variable | DIVLTQSPATLSLSPGERATLSCRASQ |
| | | | | | SVDYDGDSYMNWYQQKPGQAPRLL |
| | | | | | IYGASNLESGIPARFSGSGSGRDFTLT |
| | | | | | ISSLEPEDFATYYCQQSIEDPWTFGG |
| | | | | | GTKLEIK |
| 128 | AT-05.01v15 | protein | Heavy | Variable | QVTLKESGPALVKPTQTLTLTCTVTG |
| | | | | | YSITGGYNWHWIRQPPGKALEWIGD |
| | | | | | IHKSGSTNYNPSLKTRLTISRDTSKN |
| | | | | | QFVLTMTNMDPVDTATYYCARDYY |
| | | | | | GFSWYFDVWGQGTTVTVSS |
| 129 | AT-05.01v16 | protein | Light | CDR1 | RASQSVDYDGDSYMN |
| 130 | AT-05.01v16 | protein | Light | CDR2 | GASNRET |
| 131 | AT-05.01v16 | protein | Light | CDR3 | QQSIEDPWT |
| 132 | AT-05.01v16 | protein | Heavy | CDR1 | GGYNWH |
| 133 | AT-05.01v16 | protein | Heavy | CDR2 | DIHKSGSTNYNPSLKT |
| 134 | AT-05.01v16 | protein | Heavy | CDR3 | DYYGFSWYFDV |
| 135 | AT-05.01v16 | protein | Light | Variable | EIVLTQSPATLSLSPGERATLSCRASQ |
| | | | | | SVDYDGDSYMNWYQQKPGQAPRLL |
| | | | | | IYGASNRETGIPARFSGSGSGRDFTLT |
| | | | | | ISSLEPEDFAVYYCQQSIEDPWTFGG |
| | | | | | GTKLEIK |
| 136 | AT-05.01v16 | protein | Heavy | Variable | QVTLKESGPALVKPTQTLTLTCTVTG |
| | | | | | YSITGGYNWHWIRQPPGKALEWIGD |
| | | | | | IHKSGSTNYNPSLKTRLTISRDTSKN |
| | | | | | QFVLTMTNMDPVDTATYYCARDYY |
| | | | | | GFSWYFDVWGQGTTVTVSS |
| 137 | AT-05.02 | protein | Light | CDR1 | RSTQSIVHNNGNTYLE |
| 138 | AT-05.02 | protein | Light | CDR2 | KVSNRFS |
| 139 | AT-05.02 | protein | Light | CDR3 | FQGSHVPYT |
| 140 | AT-05.02 | protein | Heavy | CDR1 | GNNMN |
| 141 | AT-05.02 | protein | Heavy | CDR2 | NIDPYYGATSYNQKFKD |
| 142 | AT-05.02 | protein | Heavy | CDR3 | STMITPFDY |
| 143 | AT-05.02 | protein | Light | Variable | DILMTQTPLSLPVSLGDQASISCRSTQ |
| | | | | | SIVHNNGNTYLEWYLQKPGQSPKLLI |
| | | | | | YKVSNRFSGVPDRFSGSGSGTDFTLK |
| | | | | | ISRVEAEDLGVYYCFQGSHVPYTFG |
| | | | | | GGTKLEIK |
| 144 | AT-05.02 | protein | Heavy | Variable | EVQLQQSGPELEKPGASVKISCKASG |
| | | | | | YSFTGNNMNWVKQSNGKSLEWIGNI |
| | | | | | DPYYGATSYNQKFKDKATLTVDKSS |
| | | | | | STAYMQLKGLTSEDSAVYYCARST |
| | | | | | MITPFDYWGQGTTLTVSS |
| 145 | AT-05.03 | protein | Light | CDR1 | RSTQSIVHNNGNTYLE |
| 146 | AT-05.03 | protein | Light | CDR2 | KVSNRFS |
| 147 | AT-05.03 | protein | Light | CDR3 | FQSSHVPYT |
| 148 | AT-05.03 | protein | Heavy | CDR1 | GNNIN |
| 149 | AT-05.03 | protein | Heavy | CDR2 | NIDPYYGATSYNQKFKG |
| 150 | AT-05.03 | protein | Heavy | CDR3 | STMITPFDY |
| 151 | AT-05.03 | protein | Light | Variable | DILMTQIPLSLPVSLGDQASISCRSTQ |
| | | | | | SIVHNNGNTYLEWYLQKPGQSPKLLI |
| | | | | | YKVSNRFSGVPDRFSGSGSGTDFTLK |
| | | | | | ISRVEAEDLGVYYCFQSSHVPYTFGG |
| | | | | | GTKLEIR |
| 152 | AT-05.03 | protein | Heavy | Variable | EVQLQQSGPELEKPGASVKISCKASG |
| | | | | | YSFTGNNINWVKQSNGKSLEWIGNI |
| | | | | | DPYYGATSYNQKFKGKATLTVDKSS |
| | | | | | NTAYMQLKGLTSEDSAVYYCARST |
| | | | | | MITPFDYWGQGTTLTVSS |
| 153 | AT-05.04 | protein | Light | CDR1 | SASQGIRNHLN |
| 154 | AT-05.04 | protein | Light | CDR2 | YTSSLHS |
| 155 | AT-05.04 | protein | Light | CDR3 | QQYSNLPWT |
| 156 | AT-05.04 | protein | Heavy | CDR1 | SGYDWN |
| 157 | AT-05.04 | protein | Heavy | CDR2 | YISYDGSNNYNPSLKN |
| 158 | AT-05.04 | protein | Heavy | CDR3 | WYYYAMDY |
| 159 | AT-05.04 | protein | Light | Variable | DIQMTQTTSSLSASLGDRVTISCSAS |
| | | | | | QGIRNHLNWYQQKPDGTVKLLIYYT |
| | | | | | SSLHSGVPSRFSGSGSGTDYSLTISNL |
| | | | | | EPEDIATYYCQQYSNLPWTFGGGTK |
| | | | | | LEIK |
| 160 | AT-05.04 | protein | Heavy | Variable | DVQLQESGPGLVKPSQSLSLTCSVTG |
| | | | | | YSITSGYDWNWIRQFPGNKLEWMG |
| | | | | | YISYDGSNNYNPSLKNRISITRDTSKS |
| | | | | | QFFLKLNSVTTEDTATYYCARWYYY |
| | | | | | AMDYWGQGTSVTVSS |
| 161 | AT-05.05 | protein | Light | CDR1 | RSSQSIVHSNGNTYLE |
| 162 | AT-05.05 | protein | Light | CDR2 | KVSNRFS |
| 163 | AT-05.05 | protein | Light | CDR3 | FQGSHVPYT |
| 164 | AT-05.05 | protein | Heavy | CDR1 | SYGMS |
| 165 | AT-05.05 | protein | Heavy | CDR2 | TISIGGSYTYYPDSVKG |
| 166 | AT-05.05 | protein | Heavy | CDR3 | HSSIYDGYYGAY |
| 167 | AT-05.05 | protein | Light | Variable | DVLMTQTPLSLPVSLGDQASISCRSS |
| | | | | | QSIVHSNGNTYLEWYLQKPGQSPKL |
| | | | | | LIYKVSNRFSGVPDRFSGSGSGTDFT |
| | | | | | LKISRVAAEDLAVYYCFQGSHVPYT |
| | | | | | FGGGTKLEIK |
| 168 | AT-05.05 | protein | Heavy | Variable | EVQLVESGGDLVKPGGSLKLSCAAS |
| | | | | | GFTFSSYGMSWVRQTPDKRLEWVA |
| | | | | | TISIGGSYTYYPDSVKGRFTISRDNAK |
| | | | | | NTLYLQLSSLKSEDTALYYCARHSSI |
| | | | | | YDGYYGAYWGQGTLVTVSA |
| 169 | AT-05.06 | protein | Light | CDR1 | RASESVDSYGNSFMH |
| 170 | AT-05.06 | protein | Light | CDR2 | LASNLES |
| 171 | AT-05.06 | protein | Light | CDR3 | QQNNEDPPT |
| 172 | AT-05.06 | protein | Heavy | CDR1 | SYAIS |
| 173 | AT-05.06 | protein | Heavy | CDR2 | SISSAGNTYYADSVKG |
| 174 | AT-05.06 | protein | Heavy | CDR3 | GRESLLLRSYYFDY |
| 175 | AT-05.06 | protein | Light | Variable | NIVLTQSPASLAVSLGQRATISCRASE |
| | | | | | SVDSYGNSFMHWYQQKPGQPPKLLI |
| | | | | | YLASNLESGVPARFSGSGSRTDFTLTI |
| | | | | | DPVEADDAATYYCQQNNEDPPTFGG |
| | | | | | GTKLEIK |
| 176 | AT-05.06 | protein | Heavy | Variable | EVKLVESGGGLVKPGGSLKVSCAAS |
| | | | | | GFTFSSYAISWVRQTPEKRLEWVASI |
| | | | | | SSAGNTYYADSVKGRFTISRDDARNI |
| | | | | | LYLQMSSLRSEDTAIYYCARGRESLL |
| | | | | | LRSYYFDYWGQGTTLTVSS |
| 177 | AT-05.07 | protein | Light | CDR1 | RSSQSIVHSNGNTYLE |
| 178 | AT-05.07 | protein | Light | CDR2 | TVSNRFS |
| 179 | AT-05.07 | protein | Light | CDR3 | FQGSHVPPT |
| 180 | AT-05.07 | protein | Heavy | CDR1 | GYNMN |
| 181 | AT-05.07 | protein | Heavy | CDR2 | NVDPYYGATSYNQKFKG |
| 182 | AT-05.07 | protein | Heavy | CDR3 | GTYGSYAMDY |
| 183 | AT-05.07 | protein | Light | Variable | DVLMTQTPLSLPVSLGDQASISCRSS |
| | | | | | QSIVHSNGNTYLEWYLQKPGQSPKV |
| | | | | | LIYTVSNRFSGVPDRFSGSGSGTDFT |
| | | | | | LKISRVEAEDLGVYYCFQGSHVPPTF |
| | | | | | GSGTRLEIK |
| 184 | AT-05.07 | protein | Heavy | Variable | EVQLQQSGPELEKPGASVKISCKASG |
| | | | | | YSFTGYNMNWVKQSNGKSLEWIGN |
| | | | | | VDPYYGATSYNQKFKGKATLTVDK |
| | | | | | SSSTAYMQLKSLTSEDSAVYYCARG |
| | | | | | TYGSYAMDYWGQGTSVTVSS |
| 185 | AT-05.08 | protein | Light | CDR1 | RSSQSILHSNGNTYLE |
| 186 | AT-05.08 | protein | Light | CDR2 | KVSNRFS |
| 187 | AT-05.08 | protein | Light | CDR3 | FQTSHVPWT |
| 188 | AT-05.08 | protein | Heavy | CDR1 | SYGMS |
| 189 | AT-05.08 | protein | Heavy | CDR2 | TINSGGSYTYYPDSVKG |
| 190 | AT-05.08 | protein | Heavy | CDR3 | HEDYEGDYYAMNY |
| 191 | AT-05.08 | protein | Light | Variable | DVLMTQSPLSLPVSLGDQASISCRSS |
| | | | | | QSILHSNGNTYLEWYLQKPGQSPKL |
| | | | | | LIYKVSNRFSGVPDRFSGSGSGTDFT |
| | | | | | LKISRVEAEDLGVYYCFQTSHVPWT |
| | | | | | FGGGTKLEIK |
| 192 | AT-05.08 | protein | Heavy | Variable | EVQLVESGGDLVKPGGSLKLSCAAS |
| | | | | | GFTFSSYGMSWVRQTPDKRLEWVA |
| | | | | | TINSGGSYTYYPDSVKGRFTISRDNA |
| | | | | | KNTLYLQMSSLKSEDTAMYYCARH |
| | | | | | EDYEGDYYAMNYWGQGSSVTVSP |
| 193 | AT-05.00 | protein | Light | CDR1 | SVDYYGTSLMQWY |
| 194 | AT-05.00 | protein | Light | CDR2 | NVESGVPA |
| 195 | AT-05.00 | protein | Light | CDR3 | QQSRKVPWT |
| 196 | AT-05.00 | protein | Heavy | CDR1 | GYSITSDYAWN |
| 197 | AT-05.00 | protein | Heavy | CDR2 | IIYSGSTNYNPSLES |
| 198 | AT-05.00 | protein | Heavy | CDR3 | ARSTYGGNYWHFDV |
| 199 | AT-05.00 | protein | Light | Variable | DIVLTQSPASLAVSLGQRATISCRASE |
| | | | | | SVDYYGTSLMQWYQQKPGQPPKLLI |
| | | | | | YAASNVESGVPARFSGSGSGTDFSLN |
| | | | | | IHPVEEDDIAMYFCQQSRKVPWTFG |
| | | | | | GGTKVEIK |
| 200 | AT-05.00 | protein | Heavy | Variable | DVQLQESGPGLVKPSQSLSLTCTVTG |
| | | | | | YSITSDYAWNWIRQFPGNKLEWMGF |
| | | | | | IIYSGSTNYNPSLESRFSITRDTSKNQF |
| | | | | | FLQLTSVTTEDTATYYCARSTYGGN |
| | | | | | YWHFDVWGAGTTVTVSS |
| 201 | AT-05.00 | DNA | Light | CDR1 | AGTGTTGACTATTATGGCACAAGTT |
| | | | | | TAATGCAGTGGTAC |
| 202 | AT-05.00 | DNA | Light | CDR2 | AACGTAGAATCTGGGGTCCCTGCC |
| 203 | AT-05.00 | DNA | Light | CDR3 | CAACAAAGTAGGAAGGTTCCTTGG |
| | | | | | ACG |
| 204 | AT-05.00 | DNA | Heavy | CDR1 | GGCTACTCAATCACCAGTGATTAT |
| | | | | | GCCTGGAAC |
| 205 | AT-05.00 | DNA | Heavy | CDR2 | ATAATCTACAGTGGTAGCACTAAC |
| | | | | | TACAACCCATCTCTCGAAAGT |
| 206 | AT-05.00 | DNA | Heavy | CDR3 | GCCAGATCGACCTACGGTGGTAAC |
| | | | | | TACTGGCACTTCGATGTC |
| 207 | AT-05.00 | DNA | Light | Variable | GACATTGTGCTCACCCAATCTCCAG |
| | | | | | CTTCTTTGGCTGTGTCTCTAGGGCA |
| | | | | | GAGAGCCACCATCTCCTGCAGAGC |
| | | | | | CAGTGAAAGTGTTGACTATTATGG |
| | | | | | CACAAGTTTAATGCAGTGGTACCA |
| | | | | | ACAGAAACCAGGACAGCCACCCAA |
| | | | | | ACTCCTCATCTATGCTGCATCCAAC |
| | | | | | GTAGAATCTGGGGTCCCTGCCAGG |
| | | | | | TTTAGTGGCAGTGGGTCTGGGACA |
| | | | | | GACTTCAGCCTCAACATCCATCCTG |
| | | | | | TGGAGGAGGATGATATTGCAATGT |
| | | | | | ATTTCTGTCAACAAAGTAGGAAGG |
| | | | | | TTCCTTGGACGTTCGGTGGAGGCA |
| | | | | | CCAAGGTGGAAATCAAA |
| 208 | AT-05.00 | DNA | Heavy | Variable | GATGTGCAACTTCAGGAGTCGGGA |
| | | | | | CCTGGCCTGGTTAAGCCTTCTCAGT |
| | | | | | CTCTGTCCCTCACCTGCACTGTCAC |
| | | | | | TGGCTACTCAATCACCAGTGATTAT |
| | | | | | GCCTGGAACTGGATCCGGCAGTTT |
| | | | | | CCAGGAAACAAACTGGAGTGGATG |
| | | | | | GGCTTCATAATCTACAGTGGTAGC |
| | | | | | ACTAACTACAACCCATCTCTCGAA |
| | | | | | AGTCGATTCTCTATCACTCGAGACA |
| | | | | | CATCCAAGAACCAGTTCTTCCTGCA |
| | | | | | ATTGACTTCTGTGACTACTGAGGAC |
| | | | | | ACAGCCACATATTACTGTGCCAGA |
| | | | | | TCGACCTACGGTGGTAACTACTGG |
| | | | | | CACTTCGATGTCTGGGGCGCAGGG |
| | | | | | ACCACGGTCACCGTCTCCTCA |
| 209 | AT-05.01v15v1 | protein | Light | CDR1 | KASQSVDYDGDSYMN |
| 210 | AT-05.01v15v1 | protein | Light | CDR2 | GASNLES |
| 211 | AT-05.01v15v1 | protein | Light | CDR3 | QQSIEDPWT |
| 212 | AT-05.01v15v1 | protein | Heavy | CDR1 | GGYNWH |
| 213 | AT-05.01v15v1 | protein | Heavy | CDR2 | DIHKSGSTNYNPSLKS |
| 214 | AT-05.01v15v1 | protein | Heavy | CDR3 | DYYGFSWYFDV |
| 215 | AT-05.01v15v1 | protein | Light | Variable | DIVLTQSPDSLAVSLGERATINCKAS |
| | | | | | QSVDYDGDSYMNWYQQKPGQPPKL |
| | | | | | LIYGASNLESGIPDRFSGSGSGRDFTL |
| | | | | | TISSLQAEDVATYYCQQSIEDPWTFG |
| | | | | | GGTKLEIK |
| 216 | AT-05.01v15v1 | protein | Heavy | Variable | QVTLKESGPALVKPTQTLTLTCTVTG |
| | | | | | YSITGGYNWHWIRQPPGKALEWMG |
| | | | | | DIHKSGSTNYNPSLKSRITISRDTSKN |
| | | | | | QFVLTMTNMDPVDTATYYCARDYY |
| | | | | | GFSWYFDVWGQGTTVTVSS |
| 217 | AT-05.01v15v2 | protein | Light | CDR1 | KASQSVDYDGDSYMN |
| 218 | AT-05.01v15v2 | protein | Light | CDR2 | GASNLES |
| 219 | AT-05.01v15v2 | protein | Light | CDR3 | QQSIEDPWT |
| 220 | AT-05.01v15v2 | protein | Heavy | CDR1 | GGYNWH |
| 221 | AT-05.01v15v2 | protein | Heavy | CDR2 | DIHKSGSTNYNPSLKS |
| 222 | AT-05.01v15v2 | protein | Heavy | CDR3 | DYYGFSWYFDV |
| 223 | AT-05.01v15v2 | protein | Light | Variable | DIVLTQSPDSLAVSLGERATINCKAS |
| | | | | | QSVDYDGDSYMNWYQQKPGQPPKL |
| | | | | | LIYGASNLESGIPDRFSGSGSGRDFTL |
| | | | | | TISSLQAEDVATYYCQQSIEDPWTFG |
| | | | | | GGTKLEIK |
| 224 | AT-05.01v15v2 | protein | Heavy | Variable | QVTLKESGPALVKPTQTLTLTCTVTG |
| | | | | | YSITGGYNWHWIRQPPGKALEWIGD |
| | | | | | IHKSGSTNYNPSLKSRITISRDTSKNQ |
| | | | | | FVLTMTNMDPVDTATYYCARDYYG |
| | | | | | FSWYFDVWGQGTTVTVSS |
| 225 | AT-05.01v15v3 | protein | Light | CDR1 | KASQSVDYDGDSYMN |
| 226 | AT-05.01v15v3 | protein | Light | CDR2 | GASNLES |
| 227 | AT-05.01v15v3 | protein | Light | CDR3 | QQSIEDPWT |
| 228 | AT-05.01v15v3 | protein | Heavy | CDR1 | GGYNWH |
| 229 | AT-05.01v15v3 | protein | Heavy | CDR2 | DIHKSGSTNYNPSLKT |
| 230 | AT-05.01v15v3 | protein | Heavy | CDR3 | DYYGFSWYFDV |
| 231 | AT-05.01v15v3 | protein | Light | Variable | DIVLTQSPDSLAVSLGERATINCKAS |
| | | | | | QSVDYDGDSYMNWYQQKPGQPPKL |
| | | | | | LIYGASNLESGIPDRFSGSGSGRDFTL |
| | | | | | TISSLQAEDVATYYCQQSIEDPWTFG |
| | | | | | GGTKLEIK |
| 232 | AT-05.01v15v3 | protein | Heavy | Variable | QVTLKESGPALVKPTQTLTLTCTVTG |
| | | | | | YSITGGYNWHWIRQPPGKALEWMG |
| | | | | | DIHKSGSTNYNPSLKTRITISRDTSKN |
| | | | | | QFVLTMTNMDPVDTATYYCARDYY |
| | | | | | GFSWYFDVWGQGTTVTVSS |
| 233 | AT-05.01v15v4 | protein | Light | CDR1 | KASQSVDYDGDSYMN |
| 234 | AT-05.01v15v4 | protein | Light | CDR2 | GASNLES |
| 235 | AT-05.01v15v4 | protein | Light | CDR3 | QQSIEDPWT |
| 236 | AT-05.01v15v4 | protein | Heavy | CDR1 | GGYNWH |
| 237 | AT-05.01v15v4 | protein | Heavy | CDR2 | DIHKSGSTNYNPSLKS |
| 238 | AT-05.01v15v4 | protein | Heavy | CDR3 | DYYGFSWYFDV |
| 239 | AT-05.01v15v4 | protein | Light | Variable | DIVLTQSPDSLAVSLGERATINCKAS |
| | | | | | QSVDYDGDSYMNWYQQKPGQPPKL |
| | | | | | LIYGASNLESGIPDRFSGSGSGRDFTL |
| | | | | | TISSLQAEDVATYYCQQSIEDPWTFG |
| | | | | | GGTKLEIK |
| 240 | AT-05.01v15v4 | protein | Heavy | Variable | QVTLKESGPALVKPTQTLTLTCTVTG |
| | | | | | YSITGGYNWHWIRQPPGKALEWMG |
| | | | | | DIHKSGSTNYNPSLKSRLTISRDTSKN |
| | | | | | QFVLTMTNMDPVDTATYYCARDYY |
| | | | | | GFSWYFDVWGQGTTVTVSS |
| 241 | AT-05.01v15v5 | protein | Light | CDR1 | KASQSVDYDGDSYMN |
| 242 | AT-05.01v15v5 | protein | Light | CDR2 | GASNLES |
| 243 | AT-05.01v15v5 | protein | Light | CDR3 | QQSIEDPWT |
| 244 | AT-05.01v15v5 | protein | Heavy | CDR1 | GGYNWH |
| 245 | AT-05.01v15v5 | protein | Heavy | CDR2 | DIHKSGSTNYNPSLKS |
| 246 | AT-05.01v15v5 | protein | Heavy | CDR3 | DYYGFSWYFDV |
| 247 | AT-05.01v15v5 | protein | Light | Variable | DIVLTQSPDSLAVSLGERATINCKAS |
| | | | | | QSVDYDGDSYMNWYQQKPGQPPKL |
| | | | | | LIYGASNLESGIPDRFSGSGSGRDFTL |
| | | | | | TISSLQAEDVATYYCQQSIEDPWTFG |
| | | | | | GGTKLEIK |
| 248 | AT-05.01v15v5 | protein | Heavy | Variable | QVTLKESGPALVKPTQTLTLTCTVTG |
| | | | | | YSITGGYNWHWIRQPPGKALEWMG |
| | | | | | DIHKSGSTNYNPSLKSRVTISRDTSK |
| | | | | | NQFVLTMTNMDPVDTATYYCARDY |
| | | | | | YGFSWYFDVWGQGTTVTVSS |
| 249 | AT-05.01v15v6 | protein | Light | CDR1 | KASQSVDYDGDSYMN |
| 250 | AT-05.01v15v6 | protein | Light | CDR2 | GASNLES |
| 251 | AT-05.01v15v6 | protein | Light | CDR3 | QQSIEDPWT |
| 252 | AT-05.01v15v6 | protein | Heavy | CDR1 | GGYNWH |
| 253 | AT-05.01v15v6 | protein | Heavy | CDR2 | DIHKSGSTNYNPSLKT |
| 254 | AT-05.01v15v6 | protein | Heavy | CDR3 | DYYGFSWYFDV |
| 255 | AT-05.01v15v6 | protein | Light | Variable | DIVLTQSPDSLAVSLGERATINCKAS |
| | | | | | QSVDYDGDSYMNWYQQKPGQPPKL |
| | | | | | LIYGASNLESGIPDRFSGSGSGRDFTL |
| | | | | | TISSLQAEDVATYYCQQSIEDPWTFG |
| | | | | | GGTKLEIK |
| 256 | AT-05.01v15v6 | protein | Heavy | Variable | QVTLKESGPALVKPTQTLTLTCTVTG |
| | | | | | YSITGGYNWHWIRQPPGKALEWIGD |
| | | | | | IHKSGSTNYNPSLKTRITISRDTSKNQ |
| | | | | | FVLTMTNMDPVDTATYYCARDYYG |
| | | | | | FSWYFDVWGQGTTVTVSS |
| 257 | AT-05.01v15v7 | protein | Light | CDR1 | KASQSVDYDGDSYMN |
| 258 | AT-05.01v15v7 | protein | Light | CDR2 | GASNLES |
| 259 | AT-05.01v15v7 | protein | Light | CDR3 | QQSIEDPWT |
| 260 | AT-05.01v15v7 | protein | Heavy | CDR1 | GGYNWH |
| 261 | AT-05.01v15v7 | protein | Heavy | CDR2 | DIHKSGSTNYNPSLKT |
| 262 | AT-05.01v15v7 | protein | Heavy | CDR3 | DYYGFSWYFDV |
| 263 | AT-05.01v15v7 | protein | Light | Variable | DIVLTQSPDSLAVSLGERATINCKAS |
| | | | | | QSVDYDGDSYMNWYQQKPGQPPKL |
| | | | | | LIYGASNLESGIPDRFSGSGSGRDFTL |
| | | | | | TISSLQAEDVATYYCQQSIEDPWTFG |
| | | | | | GGTKLEIK |
| 264 | AT-05.01v15v7 | protein | Heavy | Variable | QVTLKESGPALVKPTQTLTLTCTVTG |
| | | | | | YSITGGYNWHWIRQPPGKALEWMG |
| | | | | | DIHKSGSTNYNPSLKTRLTISRDTSK |
| | | | | | NQFVLTMTNMDPVDTATYYCARDY |
| | | | | | YGFSWYFDVWGQGTTVTVSS |
| 265 | AT-05.01v15v8 | protein | Light | CDR1 | KASQSVDYDGDSYMN |
| 266 | AT-05.01v15v8 | protein | Light | CDR2 | GASNLES |
| 267 | AT-05.01v15v8 | protein | Light | CDR3 | QQSIEDPWT |
| 268 | AT-05.01v15v8 | protein | Heavy | CDR1 | GGYNWH |
| 269 | AT-05.01v15v8 | protein | Heavy | CDR2 | DIHKSGSTNYNPSLKT |
| 270 | AT-05.01v15v8 | protein | Heavy | CDR3 | DYYGFSWYFDV |
| 271 | AT-05.01v15v8 | protein | Light | Variable | DIVLTQSPDSLAVSLGERATINCKAS |
| | | | | | QSVDYDGDSYMNWYQQKPGQPPKL |
| | | | | | LIYGASNLESGIPDRFSGSGSGRDFTL |
| | | | | | TISSLQAEDVATYYCQQSIEDPWTFG |
| | | | | | GGTKLEIK |
| 272 | AT-05.01v15v8 | protein | Heavy | Variable | QVTLKESGPALVKPTQTLTLTCTVTG |
| | | | | | YSITGGYNWHWIRQPPGKALEWMG |
| | | | | | DIHKSGSTNYNPSLKTRVTISRDTSK |
| | | | | | NQFVLTMTNMDPVDTATYYCARDY |
| | | | | | YGFSWYFDVWGQGTTVTVSS |
| 273 | AT-05.01v15v9 | protein | Light | CDR1 | KASQSVDYDGDSYMN |
| 274 | AT-05.01v15v9 | protein | Light | CDR2 | GASNLES |
| 275 | AT-05.01v15v9 | protein | Light | CDR3 | QQSIEDPWT |
| 276 | AT-05.01v15v9 | protein | Heavy | CDR1 | GGYNWH |
| 277 | AT-05.01v15v9 | protein | Heavy | CDR2 | DIHKSGSTNYNPSLKS |
| 278 | AT-05.01v15v9 | protein | Heavy | CDR3 | DYYGFSWYFDV |
| 279 | AT-05.01v15v9 | protein | Light | Variable | DIVLTQSPDSLAVSLGERATINCKAS |
| | | | | | QSVDYDGDSYMNWYQQKPGQPPKL |
| | | | | | LIYGASNLESGIPDRFSGSGSGRDFTL |
| | | | | | TISSLQAEDVATYYCQQSIEDPWTFG |
| | | | | | GGTKLEIK |
| 280 | AT-05.01v15v9 | protein | Heavy | Variable | QVTLKESGPALVKPTQTLTLTCTVTG |
| | | | | | YSITGGYNWHWIRQPPGKALEWIGD |
| | | | | | IHKSGSTNYNPSLKSRLTISRDTSKNQ |
| | | | | | FVLTMTNMDPVDTATYYCARDYYG |
| | | | | | FSWYFDVWGQGTTVTVSS |
| 281 | AT-05.01v15v10 | protein | Light | CDR1 | KASQSVDYDGDSYMN |
| 282 | AT-05.01v15v10 | protein | Light | CDR2 | GASNLES |
| 283 | AT-05.01v15v10 | protein | Light | CDR3 | QQSIEDPWT |
| 284 | AT-05.01v15v10 | protein | Heavy | CDR1 | GGYNWH |
| 285 | AT-05.01v15v10 | protein | Heavy | CDR2 | DIHKSGSTNYNPSLKS |
| 286 | AT-05.01v15v10 | protein | Heavy | CDR3 | DYYGFSWYFDV |
| 287 | AT-05.01v15v10 | protein | Light | Variable | DIVLTQSPDSLAVSLGERATINCKAS |
| | | | | | QSVDYDGDSYMNWYQQKPGQPPKL |
| | | | | | LIYGASNLESGIPDRFSGSGSGRDFTL |
| | | | | | TISSLQAEDVATYYCQQSIEDPWTFG |
| | | | | | GGTKLEIK |
| 288 | AT-05.01v15v10 | protein | Heavy | Variable | QVTLKESGPALVKPTQTLTLTCTVTG |
| | | | | | YSITGGYNWHWIRQPPGKALEWIGD |
| | | | | | IHKSGSTNYNPSLKSRVTISRDTSKN |
| | | | | | QFVLTMTNMDPVDTATYYCARDYY |
| | | | | | GFSWYFDVWGQGTTVTVSS |
| 289 | AT-05.01v15v11 | protein | Light | CDR1 | KASQSVDYDGDSYMN |
| 290 | AT-05.01v15v11 | protein | Light | CDR2 | GASNLES |
| 291 | AT-05.01v15v11 | protein | Light | CDR3 | QQSIEDPWT |
| 292 | AT-05.01v15v11 | protein | Heavy | CDR1 | GGYNWH |
| 293 | AT-05.01v15v11 | protein | Heavy | CDR2 | DIHKSGSTNYNPSLKS |
| 294 | AT-05.01v15v11 | protein | Heavy | CDR3 | DYYGFSWYFDV |
| 295 | AT-05.01v15v11 | protein | Light | Variable | DIVLTQSPDSLAVSLGERATINCKAS |
| | | | | | QSVDYDGDSYMNWYQQKPGQPPKL |
| | | | | | LIYGASNLESGIPDRESGSGSGRDFTL |
| | | | | | TISSLQAEDVATYYCQQSIEDPWTFG |
| | | | | | GGTKLEIK |
| 296 | AT-05.01v15v11 | protein | Heavy | Variable | QVQLQESGPGLVKPSQTLSLTCTVTG |
| | | | | | YSITGGYNWHWIRQPPGKGLEWMG |
| | | | | | DIHKSGSTNYNPSLKSRITISRDTSKN |
| | | | | | QFSLKLSSVTAADTAVYYCARDYYG |
| | | | | | FSWYFDVWGQGTTVTVSS |
| 297 | AT-05.01v15v12 | protein | Light | CDR1 | KASQSVDYDGDSYMN |
| 298 | AT-05.01v15v12 | protein | Light | CDR2 | GASNRES |
| 299 | AT-05.01v15v12 | protein | Light | CDR3 | QQSIEDPWT |
| 300 | AT-05.01v15v12 | protein | Heavy | CDR1 | GGYNWH |
| 301 | AT-05.01v15v12 | protein | Heavy | CDR2 | DIHKSGSTNYNPSLKS |
| 302 | AT-05.01v15v12 | protein | Heavy | CDR3 | DYYGFSWYFDV |
| 303 | AT-05.01v15v12 | protein | Light | Variable | DIVLTQSPDSLAVSLGERATINCKAS |
| | | | | | QSVDYDGDSYMNWYQQKPGQPPKL |
| | | | | | LIYGASNRESGVPDRFSGSGSGRDFT |
| | | | | | LTISSLQAEDVAVYYCQQSIEDPWTF |
| | | | | | GGGTKLEIK |
| 304 | AT-05.01v15v12 | protein | Heavy | Variable | QVQLQESGPGLVKPSQTLSLTCTVTG |
| | | | | | YSITGGYNWHWIRQPPGKGLEWIGD |
| | | | | | IHKSGSTNYNPSLKSRVTISRDTSKN |
| | | | | | QFSLKLSSVTAADTAVYYCARDYYG |
| | | | | | FSWYFDVWGQGTTVTVSS |
| 305 | AT-05.01v15v13 | protein | Light | CDR1 | RASQSVDYDGDSYMN |
| 306 | AT-05.01v15v13 | protein | Light | CDR2 | GASNLES |
| 307 | AT-05.01v15v13 | protein | Light | CDR3 | QQSIEDPWT |
| 308 | AT-05.01v15v13 | protein | Heavy | CDR1 | GGYNWH |
| 309 | AT-05.01v15v13 | protein | Heavy | CDR2 | DIHKSGSTNYNPSLKT |
| 310 | AT-05.01v15v13 | protein | Heavy | CDR3 | DYYGFSWYFDV |
| 311 | AT-05.01v15v13 | protein | Light | Variable | DIVLTQSPATLSLSPGERATLSCRASQ |
| | | | | | SVDYDGDSYMNWYQQKPGQAPRLL |
| | | | | | IYGASNLESGIPARFSGSGSGRDFTLT |
| | | | | | ISSLEPEDFATYYCQQSIEDPWTFGG |
| | | | | | GTKLEIK |
| 312 | AT-05.01v15v13 | protein | Heavy | Variable | QVTLKESGPALVKPTQTLTLTCTVTG |
| | | | | | YSITGGYNWHWIRQPPGKALEWMG |
| | | | | | DIHKSGSTNYNPSLKTRITISRDTSKN |
| | | | | | QFVLTMTNMDPVDTATYYCARDYY |
| | | | | | GFSWYFDVWGQGTTVTVSS |
| 313 | AT-05.01v15v14 | protein | Light | CDR1 | RASQSVDYDGDSYMN |
| 314 | AT-05.01v15v14 | protein | Light | CDR2 | GASNRET |
| 315 | AT-05.01v15v14 | protein | Light | CDR3 | QQSIEDPWT |
| 316 | AT-05.01v15v14 | protein | Heavy | CDR1 | GGYNWH |
| 317 | AT-05.01v15v14 | protein | Heavy | CDR2 | DIHKSGSTNYNPSLKT |
| 318 | AT-05.01v15v14 | protein | Heavy | CDR3 | DYYGFSWYFDV |
| 319 | AT-05.01v15v14 | protein | Light | Variable | EIVLTQSPATLSLSPGERATLSCRASQ |
| | | | | | SVDYDGDSYMNWYQQKPGQAPRLL |
| | | | | | IYGASNRETGIPARFSGSGSGRDFTLT |
| | | | | | ISSLEPEDFAVYYCQQSIEDPWTFGG |
| | | | | | GTKLEIK |
| 320 | AT-05.01v15v14 | protein | Heavy | Variable | QVTLKESGPALVKPTQTLTLTCTVTG |
| | | | | | YSITGGYNWHWIRQPPGKALEWIGD |
| | | | | | IHKSGSTNYNPSLKTRLTISRDTSKN |
| | | | | | QFVLTMTNMDPVDTATYYCARDYY |
| | | | | | GFSWYFDVWGQGTTVTVSS |
| 321 | AT-05.01v15v15 | protein | Light | CDR1 | KASQSVDYDGDSYMN |
| 322 | AT-05.01v15v15 | protein | Light | CDR2 | GASNLES |
| 323 | AT-05.01v15v15 | protein | Light | CDR3 | QQSIEDPWT |
| 324 | AT-05.01v15v15 | protein | Heavy | CDR1 | GGYNWH |
| 325 | AT-05.01v15v15 | protein | Heavy | CDR2 | DIHKSGSTNYNPSLKS |
| 326 | AT-05.01v15v15 | protein | Heavy | CDR3 | DYYGFSWYFDV |
| 327 | AT-05.01v15v15 | protein | Light | Variable | DIVLTQSPASLAVSPGERATITCKAS |
| | | | | | QSVDYDGDSYMNWYQQKPGQPPKL |
| | | | | | LIYGASNLESGVPDRFSGSGSGRDFT |
| | | | | | LTISRVEAEDVAVYYCQQSIEDPWTF |
| | | | | | GQGTKVEIK |
| 328 | AT-05.01v15v15 | protein | Heavy | Variable | QVQLQESGPGLVKPSQTLSLTCTVSG |
| | | | | | YSITGGYNWHWIRQPPGKGLEWMG |
| | | | | | DIHKSGSTNYNPSLKSRITISRDTSKN |
| | | | | | QFSLKLSSVTAADTAVYYCARDYYG |
| | | | | | FSWYFDVWGRGTLVTVSS |
| 329 | AT-05.01v15v16 | protein | Light | CDR1 | KASQSVDYDGDSYLA |
| 330 | AT-05.01v15v16 | protein | Light | CDR2 | GASNRAS |
| 331 | AT-05.01v15v16 | protein | Light | CDR3 | QQSIEDPWT |
| 332 | AT-05.01v15v16 | protein | Heavy | CDR1 | GGYNWH |
| 333 | AT-05.01v15v16 | protein | Heavy | CDR2 | WIHKSGSTNYLPSLQS |
| 334 | AT-05.01v15v16 | protein | Heavy | CDR3 | DYYGFSWYFDV |
| 335 | AT-05.01v15v16 | protein | Light | Variable | EIVLTQSPATLAVSLGQRATLSCKAS |
| | | | | | QSVDYDGDSYLAWYQQKPGQPPRL |
| | | | | | LIYGASNRASGIPARFSGSGSGRDFTL |
| | | | | | NIHGVEEEDFATYYCQQSIEDPWTFG |
| | | | | | QGTKLEIK |
| 336 | AT-05.01v15v16 | protein | Heavy | Variable | DVQLVESGPEAKKPGASLSLTCKVT |
| | | | | | GYSITGGYNWHWIRQAPGNKLEWM |
| | | | | | GWIHKSGSTNYLPSLQSRISITRDTSI |
| | | | | | NTFFMELNSVTTEDTAVYYCARDYY |
| | | | | | GFSWYFDVWGAGTTVTVSS |
| 337 | AT-05.01v15v17 | protein | Light | CDR1 | SGDQSVDYDGDSYAH |
| 338 | AT-05.01v15v17 | protein | Light | CDR2 | GASKRPS |
| 339 | AT-05.01v15v17 | protein | Light | CDR3 | QQSIEDPWT |
| 340 | AT-05.01v15v17 | protein | Heavy | CDR1 | GGYNWH |
| 341 | AT-05.01v15v17 | protein | Heavy | CDR2 | DIHKSGSTNYNPSLKS |
| 342 | AT-05.01v15v17 | protein | Heavy | CDR3 | DYYGFSWYFDV |
| 343 | AT-05.01v15v17 | protein | Light | Variable | SIELTQPPSSVSVSPGQTARITCSGDQ |
| | | | | | SVDYDGDSYAHWYQQKPGQAPVLL |
| | | | | | IYGASKRPSGIPERFSGSGSGRTFTLTI |
| | | | | | SGVQAEDEADYYCQQSIEDPWTFGG |
| | | | | | GTKLTVL |
| 344 | AT-05.01v15v17 | protein | Heavy | Variable | QVQLQESGPGLVKPSQTLSLTCTVTG |
| | | | | | YSITGGYNWHWIRQFPGKKLEWMG |
| | | | | | DIHKSGSTNYNPSLKSRISITRDTSKN |
| | | | | | QFFLQLNSVTAEDTATYYCARDYYG |
| | | | | | FSWYFDVWGAGTTVTVSS |

What is claimed is:

1. An anti-V-domain Ig suppressor of T-Cell Activation (VISTA) antibody comprising a light chain variable domain and a heavy chain variable domain,
wherein said light chain variable domain comprises:
a light chain CDR1, a CDR2, and a CDR3 as set forth in Table 5; and
wherein said heavy chain variable domain comprises:
a heavy chain CDR1, a CDR2, and a CDR3 as set forth in Table 5.

2. The antibody of claim 1, wherein said light chain variable domain shares at least 80%, 85%, 90%, 91%, 92%, 93%, 94%, 95%, 96%, 97%, 98%, 99%, or 100% sequence identity with a light chain variable domain as set forth in Table 5.

3. The antibody of claim 1, wherein said heavy chain variable domain shares at least 80%, 85%, 90%, 91%, 92%, 93%, 94%, 95%, 96%, 97%, 98%, 99%, or 100% sequence identity with a heavy chain variable domain as set forth in Table 5.

4. The antibody of claim 1, wherein said light chain variable domain comprises shares at least 80%, 85%, 90%, 91%, 92%, 93%, 94%, 95%, 96%, 97%, 98%, 99%, or 100% sequence identity with the sequence selected from SEQ ID NO:7, SEQ ID NO:15, SEQ ID NO:23, SEQ ID NO:31, SEQ ID NO:39, SEQ ID NO:47, SEQ ID NO:55, SEQ ID NO:63, SEQ ID NO:71, SEQ ID NO:79, SEQ ID NO: 87, SEQ ID NO:95, SEQ ID NO:103, SEQ ID NO:111, SEQ ID NO:119, SEQ ID NO: 127, SEQ ID NO: 135, SEQ ID NO:143, SEQ ID NO:151, SEQ ID NO:159, SEQ ID NO:167, SEQ ID NO:175, SEQ ID NO: 183, SEQ ID NO: 191, SEQ ID NO: 199, SEQ ID NO:207, SEQ ID NO:215, SEQ ID NO: 223, SEQ ID NO:231, SEQ ID NO:239, SEQ ID NO:247, SEQ ID NO:255, SEQ ID NO:263, SEQ ID NO: 271, SEQ ID NO:279, SEQ ID NO:287, SEQ ID NO:295, SEQ ID NO:303, SEQ ID NO:311, SEQ ID NO:319, SEQ ID NO:327, SEQ ID NO:335, or SEQ ID NO:343.

5. The antibody of claim 1, wherein said heavy chain variable domain shares at least 80%, 85%, 90%, 91%, 92%, 93%, 94%, 95%, 96%, 97%, 98%, 99%, or 100% sequence identity with the sequence selected from SEQ ID NO:8, SEQ ID NO:16, SEQ ID NO:24, SEQ ID NO:32, SEQ ID NO: 40, SEQ ID NO:48, SEQ ID NO:56, SEQ ID NO:64, SEQ ID NO:72, SEQ ID NO:80, SEQ ID NO: 88, SEQ ID NO:96, SEQ ID NO: 104, SEQ ID NO:112, SEQ ID NO:120, SEQ ID NO:128, SEQ ID NO: 136, SEQ ID NO:144, SEQ ID NO: 152, SEQ ID NO: 160, SEQ ID NO: 168, SEQ ID NO: 176, SEQ ID NO: 184, SEQ ID NO: 192, SEQ ID NO:200, SEQ ID NO:208, SEQ ID NO:216, SEQ ID NO:224, SEQ ID NO:232, SEQ ID NO:240, SEQ ID NO:248, SEQ ID NO:256, SEQ ID NO:264, SEQ ID NO: 272, SEQ ID NO:280, SEQ ID NO:288, SEQ ID NO:296, SEQ ID NO:304, SEQ ID NO:312, SEQ ID NO: 320, SEQ ID NO:328, SEQ ID NO:336, or SEQ ID NO:344.

6. The antibody of claim 1, wherein said antibody is a humanized antibody.

7. The antibody of claim 1, wherein said antibody is a chimeric antibody.

8. The antibody of claim 1, wherein said antibody is an IgG.

9. The antibody of claim 8, wherein said IgG is an IgG1.

10. The antibody of claim 1, wherein said antibody is a Fab′ fragment.

11. The antibody of claim 1, wherein said antibody is a single chain antibody (scFv).

12. The antibody of claim 1, wherein said light chain variable domain and said heavy chain variable domain form part of a scFv.

13. The antibody of claim 1, wherein said antibody is bound to VISTA.

14. The antibody of claim 13, wherein said VISTA is part of a cell.

15. The antibody of claim 14, wherein said cell is selected from a lymphoid cell, a myeloid cell, or a stem cell.

16. The antibody of claim 14, wherein said cell is a lymphoid cell selected from a B cell or a T cell.

17. An anti-VISTA antibody, wherein the anti-VISTA antibody binds the same epitope as an antibody comprising: a heavy chain variable region domain comprising a CDR1, a CDR2, and a CDR3 as set forth in Table 5, and a light chain variable domain comprising a CDR1, a CDR2, and a CDR3 as set forth in Table 5.

18. The anti-VISTA antibody of claim 17, wherein the light chain variable domain comprises an amino acid sequence with at least 80%, 85%, 90%, 91%, 92%, 93%, 94%, 95%, 96%, 97%, 98%, 99%, or 100% sequence identity with the sequence of a light chain variable domain sequence as set forth in Table 5.

19. The anti-VISTA antibody of claim 17, wherein the heavy chain variable domain comprises an amino acid sequence with at least 80%, 85%, 90%, 91%, 92%, 93%, 94%, 95%, 96%, 97%, 98%, 99%, or 100% sequence identity with the sequence of a heavy chain variable domain sequence as set forth in Table 5.

20. An isolated nucleic acid encoding an antibody of any one of claims 1-19.

21. A cell comprising said isolated nucleic acid of claim 20.

22. A pharmaceutical composition comprising a therapeutically effective amount of an antibody of any one of claims 1-19 and a pharmaceutically acceptable excipient.

23. The pharmaceutical composition of claim 22, wherein said pharmaceutical composition is formulated in an aqueous solution at a concentration of 0.200 mg/mL to 2.00 mg/mL, 0.500 mg/mL to 1.50 mg/mL, 0.700 mg/mL to 0.875 mg/mL, or 0.875 mg/mL to 1.25 mg/mL.

24. The pharmaceutical composition of claim 22, wherein said pharmaceutical composition is formulated for intratumoral delivery.

25. The pharmaceutical composition of claim 22, further comprising a therapeutically effective amount of an antineoplastic agent.

26. The pharmaceutical composition of claim 25, wherein the antineoplastic agent is a PD-1 pathway inhibitor.

27. The pharmaceutical composition of claim 25, wherein the antineoplastic agent is selected from pembrolizumab, nivolumab, cemiplimab, tislelizumab, atezolizumab, durvalumab, avelumab, dostarlimab, retifanlimab, or toripalimab.

28. A method of treating a subject in need thereof, said method comprising administering to a subject a therapeutically effective amount of the pharmaceutical composition of claim 22.

29. The method of claim 28, further comprising administering a therapeutically effective amount of an antineoplastic agent to the subject.

30. The method of claim 29, wherein the antineoplastic agent is a PD-1 pathway inhibitor.

31. The method of claim 29, wherein the antineoplastic agent is selected from pembrolizumab, nivolumab, cemiplimab, tislelizumab, atezolizumab, durvalumab, avelumab, dostarlimab, retifanlimab, or toripalimab.

32. The method of claim 28, wherein the effective amount of an antibody and the effective amount of said antineoplastic agent are a combined synergistic amount.

33. The method of claim 28, wherein said subject has a tumor or a cancer, is at risk of developing the tumor or the cancer, or is suspected of having said tumor or said cancer.

34. The method of claim 33, wherein said cancer is selected from non-small cell lung cancer, breast cancer, ovarian cancer, renal cancer, colorectal cancer, pancreatic cancer, or a glioblastoma.

35. The method of claim 28, wherein said antibody is administered to said subject at a concentration of 0.200 mg/mL to 2.00 mg/mL, 0.500 mg/mL to 1.50 mg/mL, 0.700 mg/mL to 0.875 mg/mL, or 0.875 mg/mL to 1.25 mg/mL.

36. Use of the antibody of any one of claims 1-19 in the manufacture of a medicament for the treatment of a tumor or cancer.
